SEQUENCE LISTING (93 sequences)

SEQ ID NO: 1 - DNA, 52 bp, synthetic construct
  misc_feature 1..52: note = Description of Sequence: Synthetic primer
  source 1..52: mol_type = other DNA
ggggacaagtttgtacaaaaaagcaggctatggggaggcattcttgttgtta

SEQ ID NO: 2 - DNA, 54 bp, synthetic construct
  misc_feature 1..54: note = Description of Sequence: Synthetic primer
  source 1..54: mol_type = other DNA
ggggaccactttgtacaagaaagctgggtaagaaaattgtccaaatgcatctgg

SEQ ID NO: 3 - DNA, 48 bp, synthetic construct
  misc_feature 1..48: note = Description of Sequence: Synthetic primer
  source 1..48: mol_type = other DNA
ggggacaagtttgtacaaaaaagcaggctatggataggagagatattg

SEQ ID NO: 4 - DNA, 49 bp, synthetic construct
  misc_feature 1..49: note = Description of Sequence: Synthetic primer
  source 1..49: mol_type = other DNA
ggggaccactttgtacaagaaagctgggtacaagtgtagatctaaactc

SEQ ID NO: 5 - DNA, 23 bp, synthetic construct
  misc_feature 1..23: note = Description of Sequence: Synthetic primer
  source 1..23: mol_type = other DNA
ttgcaacagaaagttcattgagt

SEQ ID NO: 6 - DNA, 25 bp, synthetic construct
  misc_feature 1..25: note = Description of Sequence: Synthetic primer
  source 1..25: mol_type = other DNA
gcagtttgtatagaaggttgttgct

SEQ ID NO: 7 - DNA, 25 bp, synthetic construct
  misc_feature 1..25: note = Description of Sequence: Synthetic primer
  source 1..25: mol_type = other DNA
actccaaaatttcatattggtggca

SEQ ID NO: 8 - DNA, 21 bp, synthetic construct
  misc_feature 1..21: note = Description of Sequence: Synthetic primer
  source 1..21: mol_type = other DNA
aggccaaggcacttccattta

SEQ ID NO: 9 - DNA, 50 bp, synthetic construct
  misc_feature 1..50: note = Description of Sequence: Synthetic primer
  source 1..50: mol_type = other DNA
ggggacaagtttgtacaaaaaagcaggcttcatggaaatggtaagcaaga

SEQ ID NO: 10 - DNA, 51 bp, synthetic construct
  misc_feature 1..51: note = Description of Sequence: Synthetic primer
  source 1..51: mol_type = other DNA
ggggaccactttgtacaagaaagctgggttctggaccttggagcagtcagt

SEQ ID NO: 11 - DNA, 49 bp, synthetic construct
  misc_feature 1..49: note = Description of Sequence: Synthetic primer
  source 1..49: mol_type = other DNA
ggggacaagtttgtacaaaaaagcaggcttcatgtccaataattctctc

SEQ ID NO: 12 - DNA, 51 bp, synthetic construct; identical to SEQ ID NO: 10
  misc_feature 1..51: note = Description of Sequence: Synthetic primer
  source 1..51: mol_type = other DNA

SEQ ID NO: 13 - DNA, 1275 bp, Nicotiana tabacum
  source 1..1275: mol_type = genomic DNA
atggggaggcattcttgttgttacaagcagaagctgaggaaaggcctctggtcccctgaggaagatgagaaacttataaatcatattagtaaatatggtcatggctgttggagttcagtccctaaactagctgggcttcagaggtgtggcaagagctgcaggctaagatggattaactacctgagacctgatttgaaaagaggaacattctcacaagaggaagagaatttgataattgaacttcatgcagttctagggaacaagtggtcacaaattgctgctagattacctggaagaacagacaatgaaatcaagaatttatggaactcttccattaaaaaaaaactaaggcaaaaagggattgatccaaacactcacaagccactttctgaggttgagaatgaagagaaggtgtcagcaatcagtaaaaacaatgagaaagcttctgaaggctccagtgaactcaatttctttgaagctcatgagaattctaactatggaattgaaacagagaaaccaaaaccatcttcagcgacgactatggaccgctattccaataatatgagtgctgctgtagccccaccaacacatgaattcttccttaataggtttgttaacacacatgaaacctccaccactagttgcaataagcctcttgacttggcaagttacctctcttttcagcacttgaattatggctcaaacattggtttgtccatgagtccagacaccagtaatattcttttcaactccaataatcccaagaattcagaaatggtttctcatcacttcaattccaacatgtcaacaaatgataatattctcacttccatttcaaactcaattctcacatctccaatagcagcagcaagtaataattcaatagggtcttttaccagcaatgggagcagtactagtattgatcaattgcaaagaaactgttctttctttgataacaatgcattctcatggggagctgcagactgtgggaaatcagaaaaagaagccaatattcatccatcagaaactgatcctgaagacatcaaatggtctgaatatttgcatacacaatttttaccaggcaattcaatccataatcatcaaataactcaagacttgtacagtgaaaaatcaggcacacaatttgcaacagaaagttcattgagtagtactacatggctgcagaaccaacagcaacaaccttctatacaaactgcagacttatataataagcattttcagagaattccagatgcatttggacaattttcttag

SEQ ID NO: 14 - DNA, 654 bp, Nicotiana tabacum
  source 1..654: mol_type = genomic DNA
atggataggagagatattgaaactcacgatttcatgaacgtggaatctttctcccaactgccctttattaggccagcaccaacaaaagaaaaggcagccattaagctttttgggaaagaattaggtggtcgtgaggaatctaaatctgtcgattccaatgaccatagtgatcaagaatcaaaagacagtactgtcaatgtctccgacaaccgaaaatttgtatgtaactattgttgtaggaatttcccaacttcacaagcactaggaggccatcaaaatgcacataaaagagaacgtcagaatgccaaaagagctcaacgtcagccaccaaattttcatattggaaatgttaatggcacttattttcatagttccaggagcagtactactagcttttatggaagtcgtcatgtttataatggtagccattattctcaagctcatactataaatggaagtcccttggccttttggagaagccattcttctcctctttctaattatgctcgtgaccgttccaagatgatagatccattgccaatgtatgttaatggaaatttgaagaccaatagcagctcgatttctttaagccgatttgggtatgaactgaaggaaggagttcaagatcatgtgagtttagatctacacttgtaa

SEQ ID NO: 15 - DNA, 648 bp, Nicotiana sp.
  source 1..648: mol_type = genomic DNA
atggataggagagatattgaaactcacgatttcatgaacgtggaatctttctcccaactgccttttattagaccagcaccactaaaagaaaagacagccattaggattttttgcaaagaattaggtggtcgtgacgcatctaaatctgtcgatgccaataatcatagtgatgatcaagaatcaaaagacagtactgtcaatgtctccgacaaccgaaaatttgtatgtaattattgttgtaggaatttcccaactccacaagcactaggaggccatcaaaatgcacataaaagagaacgtcagaatgccaaaagagctcaacgtcagactccaaaatttcatattggtggcacttatttacgtagttcctggagcagtactactcgcttttatggaagtcgtcatgtttataatggtagccattattctcaagctcatactataaatggaagtgccttggccttttggagaagtcattcttctcctctttctaattatgctcgtgacccttccaagatgatagatccattgccagtgtatgttaatggaaatttgaacatcaataacagctcgatttctgtaagccgatttgggtatgaacagaagcaagaagttcaagatcatgtgagtttagatctacacttgtaa

SEQ ID NO: 16 - DNA, 345 bp, Nicotiana tabacum
  source 1..345: mol_type = genomic DNA
atggaaatggtaagcaagattgcatgtttcgtggttttgtgcatggtggtggttgcaccccatgcagaggcactgacctgcggccaggttcagtctagcctggctccttgcgtcccttatttgctgggccgcggccctttggggggttgttgtggcggcgttaaacgtctgttgggtgctgctcgcaccccagcggaccgaaagactgcatgcaattgcctgaaatcagccgctaatacttttaagggcattgatatgggcaacgccgctcgtctccctggtacttgtggcgttaacattccctacaagatcagtccctccactgactgctccaaggtccagtga

SEQ ID NO: 17 - DNA, 489 bp, Nicotiana tabacum
  source 1..489: mol_type = genomic DNA
atgtccaataattctctctctataaaaaaccctaaggtctcactagcttttgtacacaacactcaaggcaataacatttgtagtactatccccctctactttgtccaaactactctctattgttctctaatttttcttttaagaatggaaatggtaagcaagattgcatgtttcgtggttttgtgcatggtggtggttgcaccccatgcagaggcactgacctgcggccaggttcagtctagcctggctccttgcgtcccttatttgctgggccgcggccctttggggggttgttgtggcggcgttaaacgtctgttgggtgctgctcgcaccccagcggaccgaaagactgcatgcaattgcctgaaatcagccgctaatacttttaagggcattgatatgggcaacgccgctcgtctccctggtacttgtggcgttaacattccctacaagatcagtccctccactgactgctccaaggtccagtga

SEQ ID NO: 18 - protein, 424 aa, Nicotiana tabacum
MGRHSCCYKQKLRKGLWSPEEDEKLINHISKYGHGCWSSVPKLAGLQRCGKSCRLRWINYLRPDLKRGTFSQEEENLIIELHAVLGNKWSQIAARLPGRTDNEIKNLWNSSIKKKLRQKGIDPNTHKPLSEVENEEKVSAISKNNEKASEGSSELNFFEAHENSNYGIETEKPKPSSATTMDRYSNNMSAAVAPPTHEFFLNRFVNTHETSTTSCNKPLDLASYLSFQHLNYGSNIGLSMSPDTSNILFNSNNPKNSEMVSHHFNSNMSTNDNILTSISNSILTSPIAAASNNSIGSFTSNGSSTSIDQLQRNCSFFDNNAFSWGAADCGKSEKEANIHPSETDPEDIKWSEYLHTQFLPGNSIHNHQITQDLYSEKSGTQFATESSLSSTTWLQNQQQQPSIQTADLYNKHFQRIPDAFGQFS

SEQ ID NO: 19 - protein, 217 aa, Nicotiana tabacum
MDRRDIETHDFMNVESFSQLPFIRPAPTKEKAAIKLFGKELGGREESKSVDSNDHSDQESKDSTVNVSDNRKFVCNYCCRNFPTSQALGGHQNAHKRERQNAKRAQRQPPNFHIGNVNGTYFHSSRSSTTSFYGSRHVYNGSHYSQAHTINGSPLAFWRSHSSPLSNYARDRSKMIDPLPMYVNGNLKTNSSSISLSRFGYELKEGVQDHVSLDLHL

SEQ ID NO: 20 - protein, 215 aa, Nicotiana sp.
MDRRDIETHDFMNVESFSQLPFIRPAPLKEKTAIRIFCKELGGRDASKSVDANNHSDDQESKDSTVNVSDNRKFVCNYCCRNFPTPQALGGHQNAHKRERQNAKRAQRQTPKFHIGGTYLRSSWSSTTRFYGSRHVYNGSHYSQAHTINGSALAFWRSHSSPLSNYARDPSKMIDPLPVYVNGNLNINNSSISVSRFGYEQKQEVQDHVSLDLHL

SEQ ID NO: 21 - protein, 114 aa, Nicotiana tabacum
MEMVSKIACFVVLCMVVVAPHAEALTCGQVQSSLAPCVPYLLGRGPLGGCCGGVKRLLGAARTPADRKTACNCLKSAANTFKGIDMGNAARLPGTCGVNIPYKISPSTDCSKVQ

SEQ ID NO: 22 - protein, 162 aa, Nicotiana tabacum
MSNNSLSIKNPKVSLAFVHNTQGNNICSTIPLYFVQTTLYCSLIFLLRMEMVSKIACFVVLCMVVVAPHAEALTCGQVQSSLAPCVPYLLGRGPLGGCCGGVKRLLGAARTPADRKTACNCLKSAANTFKGIDMGNAARLPGTCGVNIPYKISPSTDCSKVQ

SEQ ID NO: 23 - DNA, 1448 bp, Nicotiana tabacum
  source 1..1448: mol_type = genomic DNA
atggggaggcattcttgttgttacaagcagaagctgaggaaaggcctctggtcccctgaggaagatgagaaacttataaatcatattagtaaatatggtcatggctgttggagttcagtccctaaactagctggtacaatacacactatattacctcaattacttttactgagtttaatttcttgaaaatgcatgaggaaaaataacactagtttgtttttgatgcagggcttcagaggtgtggcaagagctgcaggctaagatggattaactacctgagacctgatttgaaaagaggaacattctcacaagaggaagagaatttgataattgaacttcatgcagttctagggaacaagtaaatacatattttcttgattcttatagtacttttcatgatgtaacaaaactgacaccttgacttctttttccataggtggtcacaaattgctgctagattacctggaagaacagacaatgaaatcaagaatttatggaactcttccattaaaaaaaaactaaggcaaaaagggattgatccaaacactcacaagccactttctgaggttgagaatgaagagaaggtgtcagcaatcagtaaaaacaatgagaaagcttctgaaggctccagtgaactcaatttctttgaagctcatgagaattctaactatggaattgaaacagagaaaccaaaaccatcttcagcgacgactatggaccgctattccaataatatgagtgctgctgtagccccaccaacacatgaattcttccttaataggtttgttaacacacatgaaacctccaccactagttgcaataagcctcttgacttggcaagttacctctcttttcagcacttgaattatggctcaaacattggtttgtccatgagtccagacaccagtaatattcttttcaactccaataatcccaagaattcagaaatggtttctcatcacttcaattccaacatgtcaacaaatgataatattctcacttccatttcaaactcaattctcacatctccaatagcagcagcaagtaataattcaatagggtcttttaccagcaatgggagcagtactagtattgatcaattgcaaagaaactgttctttctttgataacaatgcattctcatggggagctgcagactgtgggaaatcagaaaaagaagccaatattcatccatcagaaactgatcctgaagacatcaaatggtctgaatatttgcatacacaatttttaccaggcaattcaatccataatcatcaaataactcaagacttgtacagtgaaaaatcaggcacacaatttgcaacagaaagttcattgagtagtactacatggctgcagaaccaacagcaacaaccttctatacaaactgcagacttatataataagcattttcagagaattccagatgcatttggacaattttcttag

SEQ ID NO: 24 - DNA, 654 bp, Nicotiana tabacum; identical to SEQ ID NO: 14
  source 1..654: mol_type = genomic DNA

SEQ ID NO: 25 - DNA, 648 bp, Nicotiana sp.; identical to SEQ ID NO: 15
  source 1..648: mol_type = genomic DNA

SEQ ID NO: 26 - DNA, 1014 bp, Nicotiana tabacum
  source 1..1014: mol_type = genomic DNA
atggaaatggtaagcaagattgcatgtttcgtggttttgtgcatggtggtggttgcaccccatgcagaggcactgacctgcggccaggttcagtctagcctggctccttgcgtcccttatttgctgggccgcggccctttggggggttgttgtggcggcgttaaacgtctgttgggtgctgctcgcaccccagcggaccgaaagactgcatgcaattgcctgaaatcagccgctaatacttttaagggcattgatatgggcaacgccgctcgtctccctggtacttgtggcgttaacattccctacaagatcagtccctccactgactgctccaagtatgtttttcactttcttttctttttattatgagtttacgatctcttcatttacattgtaaagaatttttgtaagatcaggttaaatttgaactacaacaagaagtaactttttatggtaacataaaaaaataaagtaatgctatcaaattataactgatatgataatgtaaaaaaacttaatctccttttgctatccttacactacccatattttcattcttacttcaattttacttttccggctccactttaattgatttttttgcccttttttatagtatagtacattatttgattataactgatatgatttttttgatatcaagaaagaactaaaaaaattttccaaagttgtccctggagtaaagagttggactatctgttatttttaatgaacaaattaaagagataataaaagttaatatgattaattttattattaattaatattaaaaagcgaaccccttaatatgagtgaaaacaaccaaataatcaattaaagtggatcggaagaagcaatcacttgaaagtggtgtaacatgatacataatcacatatttatacgacactgaattggctggtaagtgagaagattattattcataaccagtgcgttttttttggtgtgcgtatggggttaatgttggttttcatgtgacaatgcagggtccagtga

SEQ ID NO: 27 - DNA, 1158 bp, Nicotiana tabacum
  source 1..1158: mol_type = genomic DNA
atgtccaataattctctctctataaaaaaccctaaggtctcactagcttttgtacacaacactcaaggcaataacatttgtagtactatccccctctactttgtccaaactactctctattgttctctaatttttcttttaagaatggaaatggtaagcaagattgcatgtttcgtggttttgtgcatggtggtggttgcaccccatgcagaggcactgacctgcggccaggttcagtctagcctggctccttgcgtcccttatttgctgggccgcggccctttggggggttgttgtggcggcgttaaacgtctgttgggtgctgctcgcaccccagcggaccgaaagactgcatgcaattgcctgaaatcagccgctaatacttttaagggcattgatatgggcaacgccgctcgtctccctggtacttgtggcgttaacattccctacaagatcagtccctccactgactgctccaagtatgtttttcactttcttttctttttattatgagtttacgatctcttcatttacattgtaaagaatttttgtaagatcaggttaaatttgaactacaacaagaagtaactttttatggtaacataaaaaaataaagtaatgctatcaaattataactgatatgataatgtaaaaaaacttaatctccttttgctatccttacactacccatattttcattcttacttcaattttacttttccggctccactttaattgatttttttgcccttttttatagtatagtacattatttgattataactgatatgatttttttgatatcaagaaagaactaaaaaaattttccaaagttgtccctggagtaaagagttggactatctgttatttttaatgaacaaattaaagagataataaaagttaatatgattaattttattattaattaatattaaaaagcgaaccccttaatatgagtgaaaacaaccaaataatcaattaaagtggatcggaagaagcaatcacttgaaagtggtgtaacatgatacataatcacatatttatacgacactgaattggctggtaagtgagaagattattattcataaccagtgcgttttttttggtgtgcgtatggggttaatgttggttttcatgtgacaatgcagggtccagtga

SEQ ID NO: 28 - DNA, 1566 bp, Cannabis sativa
  source 1..1566: mol_type = genomic DNA
atggggaggcactcttgttgttacaagcagaaactgagaaaagggttgtggtcaccagaagaagatgagaaacttcttaattatataaccaagcatggacatggctgctggagctctgtccctaagctagctggtcttcagagatgtggaaaaagttgcaggctaaggtggataaattatttgaggcctgatttgaaaagaggcccattttcacaacaagaggagaatttgataattgaacttcatgcagttcttggcaacagatggtcacagattgcagctcagttaccaggaagaacagataatgagattaaaaacttatggaattcttgcattaagaagaaactgaggcaaaaagggattgacccaaatactcataagccattatctgaggtagaaaatgacattggtaataaattggagaacaagggtaacaaagctgcaaccaataacaacaacaatgagaatattaataattctactgttagagcttcttcattaggaaacttatccaatgatcatcatcatcatcatcatcatcatctgaatctagctgaccagtcacaaccatcaatggcggccatcaatcgttacccactattggaagtctcatcctcaactccgccgacacaagaattcttcatagaaaaatcaacagataccagatcatcaccatcaatatcatcatcatcaccttgtgatttttctacctacttctctttccactcaaacaattacaatacgacgtcgtccgctgctgcagctgctgctgtttctcatcatcaagatcaaaacaacaacaacaacatggccagtttctgcttcaacattaatcaaaattcaactagacctccacaacaccatcatcataatcagatgattagtaatctcatccagccactacaacaacaagtatcaccttcatcaacaacaacagcatcatcatcatcaccaccctccaatattccacgtgtaaagccctccataagtctccccttattatctgatcaccaaaacaacagtaatagcactactactactactacaacaactactggagccgtacaaaattgggaaactagtactttcagcaacaacggaagtagtagtagtagctgcaatatcgaattacaaggtaataataataacaacaacaacaacttctttgatcacaacactaattccaccgccgcggccgccgccgccgccgctcctaataacttctcgtggggattagtcaatgaaagtactgttggtagcataaaatctgatgacccagaagacataaaatggtctgaatatctccatagcccttttcttcttggtggaggaattagtaatactaataatcaaaattcttcttcttcttcacatcttcaacccattttgtacagtaacattgtgaaaccagaatcacactttagtaatactactactgctacaggatcaaaccccacgtggcatcatcagaacgatcatcatcagctacaagcggcttcatcagaaataatgtacactaataaagatctacagagacttgctgtagcttttggacagaccctttag

SEQ ID NO: 29 - DNA, 705 bp, Cannabis sativa
  source 1..705: mol_type = genomic DNA
atggcggagctagaataccaaattaccaaaaacccaactccaaccaccacgcgcttaaaactattcggcttcaacgtacaagacgactacgaagaagccgtagctgactcagcaaaaacgacgccgtctggttcaccggaatccggcaacggatttcagaactccggtgaccggaaatacgagtgtcagtactgttgtagagaattcgctaattcccaagccctaggtggccatcaaaacgctcataaaaaagaaagacaacagcttaaacgagctcagcttcaagcgagccgaaacgccgccgtttcttacgctagaaacccgataatctcggcttttgcgccgccgccgcatcttctttcgccagctggacaggtaatggtaccggcggcttctccttcgtgggtttatgttccgcgttcggctcctcctttccacgtgtcgcatggctgtgtgtttcctcctgtgatgaacggtgggagaggtgcgagttcagttgggttatcgtatggtagcgatgtaggggataattctacattgacgatgggttctcaggttcaacaacaacaacaacactcacgggcccataatttaagggttgatgggccttcgttgagtagattttcgaaaggagatggtgggcccaattttgatgatgcattgggcctggacttgcatcttagtcttgctcccgctgctccatga

SEQ ID NO: 30 - DNA, 357 bp, Cannabis sativa
  source 1..357: mol_type = genomic DNA
atggcctcaaactcttcaagcatgaagctactctgcatggtggtcatggtcatggtggtagttgcaccaacgacacatgccctaacatgtggccaagtgtctagcagtcttagcccatgcatcaactacctcaagagcggcggtgctgtcccttcaccatgttgtaatgggatcaggtcgctgaacagtgcggccaagaccccagcggaccgcaagactgcatgcaagtgcttgcagtctgcggccggcagcatcaaaggactcaacttaaacctcgccgcgggacttcctggaaagtgtggtgtcaatgttccatacaagatcagtccctccaccaattgcaacagtgtgcagtga

SEQ ID NO: 31 - protein, 521 aa, Cannabis sativa
MGRHSCCYKQKLRKGLWSPEEDEKLLNYITKHGHGCWSSVPKLAGLQRCGKSCRLRWINYLRPDLKRGPFSQQEENLIIELHAVLGNRWSQIAAQLPGRTDNEIKNLWNSCIKKKLRQKGIDPNTHKPLSEVENDIGNKLENKGNKAATNNNNNENINNSTVRASSLGNLSNDHHHHHHHHLNLADQSQPSMAAINRYPLLEVSSSTPPTQEFFIEKSTDTRSSPSISSSSPCDFSTYFSFHSNNYNTTSSAAAAAAVSHHQDQNNNNNMASFCFNINQNSTRPPQHHHHNQMISNLIQPLQQQVSPSSTTTASSSSPPSNIPRVKPSISLPLLSDHQNNSNSTTTTTTTTTGAVQNWETSTFSNNGSSSSSCNIELQGNNNNNNNNFFDHNTNSTAAAAAAAAPNNFSWGLVNESTVGSIKSDDPEDIKWSEYLHSPFLLGGGISNTNNQNSSSSSHLQPILYSNIVKPESHFSNTTTATGSNPTWHHQNDHHQLQAASSEIMYTNKDLQRLAVAFGQTL

SEQ ID NO: 32 - protein, 234 aa, Cannabis sativa
MAELEYQITKNPTPTTTRLKLFGFNVQDDYEEAVADSAKTTPSGSPESGNGFQNSGDRKYECQYCCREFANSQALGGHQNAHKKERQQLKRAQLQASRNAAVSYARNPIISAFAPPPHLLSPAGQVMVPAASPSWVYVPRSAPPFHVSHGCVFPPVMNGGRGASSVGLSYGSDVGDNSTLTMGSQVQQQQQHSRAHNLRVDGPSLSRFSKGDGGPNFDDALGLDLHLSLAPAAP

SEQ ID NO: 33 - protein, 118 aa, Cannabis sativa
MASNSSSMKLLCMVVMVMVVVAPTTHALTCGQVSSSLSPCINYLKSGGAVPSPCCNGIRSLNSAAKTPADRKTACKCLQSAAGSIKGLNLNLAAGLPGKCGVNVPYKISPSTNCNSVQ

SEQ ID NO: 34 - DNA, 1768 bp, Cannabis sativa
  source 1..1768: mol_type = genomic DNA
atggggaggcactcttgttgttacaagcagaaactgagaaaagggttgtggtcaccagaagaagatgagaaacttcttaattatataaccaagcatggacatggctgctggagctctgtccctaagctagctggtattaattctactattactactatcaatgctaataatttctgtatgtttgcatcaaaattagttacatatttattcatcattaattttttatgacaggtcttcagagatgtggaaaaagttgcaggctaaggtggataaattatttgaggcctgatttgaaaagaggcccattttcacaacaagaggagaatttgataattgaacttcatgcagttcttggcaacaggtaattaattacttgtttcttgagaaaaaagaagaatgacattaattttgagttgaaattcatttctgggtacttatgtaaataattttgtgcaaatttttcagatggtcacagattgcagctcagttaccaggaagaacagataatgagattaaaaacttatggaattcttgcattaagaagaaactgaggcaaaaagggattgacccaaatactcataagccattatctgaggtagaaaatgacattggtaataaattggagaacaagggtaacaaagctgcaaccaataacaacaacaatgagaatattaataattctactgttagagcttcttcattaggaaacttatccaatgatcatcatcatcatcatcatcatcatctgaatctagctgaccagtcacaaccatcaatggcggccatcaatcgttacccactattggaagtctcatcctcaactccgccgacacaagaattcttcatagaaaaatcaacagataccagatcatcaccatcaatatcatcatcatcaccttgtgatttttctacctacttctctttccactcaaacaattacaatacgacgtcgtccgctgctgcagctgctgctgtttctcatcatcaagatcaaaacaacaacaacaacatggccagtttctgcttcaacattaatcaaaattcaactagacctccacaacaccatcatcataatcagatgattagtaatctcatccagccactacaacaacaagtatcaccttcatcaacaacaacagcatcatcatcatcaccaccctccaatattccacgtgtaaagccctccataagtctccccttattatctgatcaccaaaacaacagtaatagcactactactactactacaacaactactggagccgtacaaaattgggaaactagtactttcagcaacaacggaagtagtagtagtagctgcaatatcgaattacaaggtaataataataacaacaacaacaacttctttgatcacaacactaattccaccgccgcggccgccgccgccgccgctcctaataacttctcgtggggattagtcaatgaaagtactgttggtagcataaaatctgatgacccagaagacataaaatggtctgaatatctccatagcccttttcttcttggtggaggaattagtaatactaataatcaaaattcttcttcttcttcacatcttcaacccattttgtacagtaacattgtgaaaccagaatcacactttagtaatactactactgctacaggatcaaaccccacgtggcatcatcagaacgatcatcatcagctacaagcggcttcatcagaaataatgtacactaataaagatctacagagacttgctgtagcttttggacagaccctttag

SEQ ID NO: 35 - DNA, 705 bp, Cannabis sativa; identical to SEQ ID NO: 29
  source 1..705: mol_type = genomic DNA

SEQ ID NO: 36 - DNA, 474 bp, Cannabis sativa
  source 1..474: mol_type = genomic DNA
atggcctcaaactcttcaagcatgaagctactctgcatggtggtcatggtcatggtggtagttgcaccaacgacacatgccctaacatgtggccaagtgtctagcagtcttagcccatgcatcaactacctcaagagcggcggtgctgtcccttcaccatgttgtaatgggatcaggtcgctgaacagtgcggccaagaccccagcggaccgcaagactgcatgcaagtgcttgcagtctgcggccggcagcatcaaaggactcaacttaaacctcgccgcgggacttcctggaaagtgtggtgtcaatgttccatacaagatcagtccctccaccaattgcaacaggtataaataattatgtataaatttttaatgattaacaattgttttatcaatctaataacataaacaaatttatgttgttacttatgataattattttatttttcttaattaatacagtgtgcagtga

SEQ ID NO: 37 - DNA, 993 bp, Artemisia annua
  source 1..993: mol_type = genomic DNA
atggccaggcattccacttgttacaaacaaaggctaagaaaaggcctttggtcaccagaagaagatgaaaaactcatcaaacacatcaccaaatttggtcacggctgttggagttctgtccctaagctcgcgggtcttcaaagatgcggaaagagttgtaggttacgatggattaattaccttaggcctgatttgaaacgaggcacattttctcaacaagaagagaccttaatcgtagagttgcatgcagtactcggcaacaaatggtctcagattgcggctcagttgccagggagaacagataacgagattaagaacttatggaactcatccattaagaagaagttaagacaacgagggattgatccaaatactcacaaaccattatcggacgtcgaaaatgaggacaaaccatcacctagaagtaacaacaaaaaccaccaacaaactattattccaagtatagagaatccatcactagagacacacgaattcttcagaaaccgatttaccacaagtcacgaaaatgccaacctcgcctcccacaccgacactaaacataacaatactgaccagttttcaggtttcctagacttcgcttacaaccaaccaccacaacctgaatcaagtctcttgtttggctctagctctaacactgacaccagtctttccaatcccttccaacctaccaattgggatacaacgacatctttatttgacgcgaataacgggtttcataaccaagttcctttagttggaaatgaaagccaaccagaagacatcaaatggaataagtatctccaatcaccatttatgtttggaggtgctacactccagagtcgggttttgtgtaatgagacaaaacccgacttgggaatgagtataaatatgaatatgaacataaataatgagttatatgggaatcatgaaggtgtggatacatacaacaagcaattacaaaggatttgtgcaagctatgggcaatttacatag

SEQ ID NO: 38 - protein, 330 aa, Artemisia annua
MARHSTCYKQRLRKGLWSPEEDEKLIKHITKFGHGCWSSVPKLAGLQRCGKSCRLRWINYLRPDLKRGTFSQQEETLIVELHAVLGNKWSQIAAQLPGRTDNEIKNLWNSSIKKKLRQRGIDPNTHKPLSDVENEDKPSPRSNNKNHQQTIIPSIENPSLETHEFFRNRFTTSHENANLASHTDTKHNNTDQFSGFLDFAYNQPPQPESSLLFGSSSNTDTSLSNPFQPTNWDTTTSLFDANNGFHNQVPLVGNESQPEDIKWNKYLQSPFMFGGATLQSRVLCNETKPDLGMSINMNMNINNELYGNHEGVDTYNKQLQRICASYGQFT

SEQ ID NO: 39 - DNA, 1485 bp, Nicotiana tabacum
  source 1..1485: mol_type = genomic DNA
atgtcggtctttgcggttatttcattctttctacttctgttttttcttttcaaatcatatttgccctcatcgaaaacaaagaaaaattctccaccatctccttcaaagcttccgttaatcggtcacttccacaaactaggcttacaacctcaccgttctctacaaaaactatcaaatgaacatggtcccatgatgatgcttcaattcggtagcgtacctgtgcttatcgcttcatcagctgaagctgcttccgaaatcatgaaaacccaagatttgtcttttgcaaacaaacccatttcaaccattcctagcaagcttttcttcggcccaaaggacgttgccttcaccccatatggggattactggaggaatgccagaagcatttgcatgcttcagcttttgaacaacaaaagagtccagtcttttcgaaagataagggaagaagagacttctcttcttctccagaggattagggaatcgccaaattcagaagtcgatttaacggagctgttcgtttccatgactaacgacatagtttgcagggtggccttaggaaggaagtattgtgatggggaagaagggaggaaattcaagtctttgctgttagagtttgtggaattgttgggagtttttaacattggagattacatgccgtggcttgcatggatgaatcgtttcaatggtttgaatgccaaagtggataaagtggcgaaagagtttgatgcatttttggaggatgtgattgaggaacacggaggaaataagaaatcagacactgaagctgaaggggcagacttcgtggatatattattgcaggttcacaaagaaaacaaggctggttttcaagtcgaaatggatgcaatcaaagctattatcatggatatgtttgctgcgggaacagatacaacttccacgcttctagagtggacaatgaacgagctcttaagaaatccaaaaacattgaataagttgagagatgaggtgagacaagtgactcaagggaagacagaggtaacagaggatgacttagagaaaatgccgtatttaagagcagcagttaaggagagttccaggctacactctccagtgccacttctacctcgagaagcaattaaggatgcaaaggttttgggctacgatatagctgcagggactcaagtcctcgtttgtccatgggcaatctcaagagatccaaacctttgggaaaatccagaggagtttcaacctgaaagattcttggatacttccatagattacaaaggcttacatttcgagttaattccattcggtgcaggtcggaggggttgccctggcatcacatttgctaagtttgtgaatgagctagcattggcaagattaatgttccattttgatttctcgctaccaaaaggagttaagcatgaggatttggacgtggaggaagctgctggaattactgttagaaggaagttcccccttttagccgtcgccactccatgctcgtga

SEQ ID NO: 40 - DNA, 966 bp, Nicotiana tabacum
  source 1..966: mol_type = genomic DNA
atggcagaaaaaatcaccagccacgagaacacaaggtatgcagtggtgacagggggaaataaaggaataggatatgaaacatgcaggcaactagcaaaggaaggaatagtggtagtgttgacagcaagggatgaaaggagaggaattgaagctctcgaaaagctcaaggaagagtactcaagcaataaaactgatgatgatcagattttatttcatcaacttgatgttatggatccagctagtatttcttctcttgtggacttcatcaaaactaaatttggaaagctcgatattctggttaacaacgcagggattggtggattaatggtagaaggagatgttgttataataaaagatttaatagaaggagacttcgtaaccatttctgctgaaaatggggaagaggatggtattaagaaatcaattgaaggtattgagcgtattgttacagattatgagttgacaaaacaatgcctggagacaaacttctatggtgcaaaaagaatgattgaagcatttattcccctccttcagctctctaactccccaagaattgttaatgtcgcttctttcttggggaagttaaagctattgtgcaaccaatgggctataggaatgctaagtgatgctaaaagcctgagagaagaaagggtggatgaagtgttgaatgaatttataaaagattttaaagagaaatcaatagaagccaaaggatggccaacttatttctcagcttacaaagtctcgaaagcatccctgattgcttacacaagggttttagctacgaaatatccaaattttcggataaattctgtgtgtcctggattttgcaaaacagacgtgaactgcaatactgggagcttaactgctgaagaaggtgctgaaagcttggtgaagcttgctttggtgccaaatgatggaccctctggtctcttcttttatagaaaggaggtcacctctttttga

SEQ ID NO: 41 - DNA, 1797 bp, Nicotiana sylvestris
  source 1..1797: mol_type = genomic DNA
atgagtcaatcaatttctccattaatctgttctcactttgcgaaatttcagtcgaatatttggagatgcaatacttctcaactcagagttatacactcatcatatgcctcttttggagggagaagaaaagagagagtaagaagaatgaatcgagcaatggatctttcttcaagctctcgtcatttggcagattttccctcaacaatttggggtgaccattttctctcctacaattctgaaataacagaaattactacccaagagaaaaatgaacatgaaatgctaaaagaaatagttcggaaaatgttggtagaaactccagataatagtacacaaaaactagtcttgattgacacaattcaaagattgggattagcatatcatttcaatgatgagattgaaaactccattcaaaacatctttaatttgtctcaaaatagtgaagatgacgatgaacacaacctttatgttgctgctcttcgttttcgacttgcgaggcaacaaggatattacatgtcttcagatgtgttcaagcaattcactaaccatgacggaaaattcaaggaaaatcatactaatgatgttcaaggattattgagtttgtatgaagcagcacatatgagagtgcacgacgaggaaattctagaagaagctcttatctttaccacgactcatctcgagtccgtgatcccgaatttgagcaactcgcttaaggtacaagttactgaagccttaagccatcctattcgcaaagctataccaagggtgggagcaaggaaatacatacacatatatgaaaacattggaacacataatgatttacttttgaaatttgcaaagttggacttcaacatgttacaaaagcttcatcgaaaagagcttaacgagctaacaagctggtggaaagatttggatcgtgcaaacaaatttccatatgcaaaggacagattagtagaagcttacttttggacggtgggaatatattttgaacctcaatatagtcgttcaagaagtttggtaacaaaagtagtcaaaatgaactccattattgatgacacttatgatgcttatgcaacttttgatgagcttgtgcttttcacggatgcgatccaaagatgggacgaaggtgccatggatttattaccgacatatctgagacctatttatcaaggccttctcgacgttttcaatgaaatggaagaagtattggccaaagaaggtaaagcagatcacatctactatgcgaaaaaagagatgaaaaaggtggcggaagtctattttaaggaagctgaatggttgaatgctaactacattccaaaatgcgaggagtatatgaaaaatggacttgtaagctctaccggtccgatgtatggaataatttctttggttgttatggaggaaattataacaaaagaggcttttgaatggttgacaaatgaacctttgattcttcgagctgcatcaacaatttgtagattaatggatgatatggctgatcatgaagttgaacaacaaagaggacatgttgcttcatttgttgagtgctacatgaaagaatatggagtttcaaagcaagaagcatatgttgagatgcggaaaaaaatcacaaatgcgtggaaagatataaataaggaactcttgcgccctactgcagtaccaatgtttatcctcgaacgatctttaaatttttcaagattggccgatacatttttgaaagatgatgatggatacacaaatcccaaatccaaagttaaagacttgattgcttcgttgtttgtcgaatctgtcgacatatga

SEQ ID NO: 42 - DNA, 1098 bp, Nicotiana tabacum
  source 1..1098: mol_type = genomic DNA
atggcatttttggctaccatttctggccatgaaaatatgcttctttccaataccctaaacaataactttattttcagtggaaaacctccacagagacattcttatagtttcctccccaagaaaatccaggccagaagtgttgcaaactcatccaaaacatttcaagtcaaagaagaagaattctcatctaagacagagaaattcatcttgcctaagtttgactttgaagaatatatgaaaatgaaggcaattaaggtaaacaaagcactagatgatgcaataccaatgcaagagcctataaaaattcatgaagccatgagatactcacttctagctgggggaaaacgcgtccggccgatcctatgcatggcttcttgtgaagtagtaggaggggatgaatccttagctattcctgcagcttgctcggttgagatgatccacaccatgtcactcatccacgacgatcttccttgcatggacaacgatgatctacgtcgtggcaagcccacgagccacaaggctttcggggaagacactgcagttctaacaggggatgcacttttgtctttggcctttgaacatgtagcttccaagactaaagatgtgacaccccaaagagtggttcaagccgttggcgaattgggttcagccgttggctcgaaagggcttgtggcggggcagattgtggacatagctagtgagggaaaacaagtgagcctaactgaattagagtacattcacaaccataagacagggaaactattggaggctgctgtggtttgtggggcaataattgggggagggaatgagattgaggtggagagaatgaggaactatgctagatgccttggactgttgtttcaagtggtagatgatattcttgatgttactaagtcatcagaagagttgggaaagacagctggtaaagacctagtgactgataaggctacatatcctaagttgatggggctagaaaaagctcgggagctcgccggagagctggtggctaaggccatggatgagctgagctactttgatgctgccaaggcggcacctctttatcattttgctaattatattgcacatcgccagaattga

SEQ ID NO: 43 - DNA, 2379 bp, Nicotiana sp.
  source 1..2379: mol_type = genomic DNA
atggtacttggactgagaagcaaaatcataccacttcctgatcataagttgggaaatatcaaattaggttcagtaaccaatgcaatttgccacagaccatgtagagtaagatgcagccacagtactgcttcatcaatggaagaggcaaaggagagaataagggaaacatttggaaaaatagagctatctccttcttcctatgacacagcatgggtagctatggtcccttcaagatattctatgaaccaaccatgttttcctcagtgcttagattggattcttgaaaatcaaagagaagatggatcttggggcctaaatcctagccatccattgcttgtaaaagactccctttcttccactctagcatctttgcttgcccttcgcaaatggagaattggagataaccaagtccaaagaggccttggctttattgaaacgcatggttgggcagtcgataacaaggatcagatttcacctttaggatttgaaattatatttccctgcatgatcaactatgcagagaaacttaatttggatctacctttggatcctaaccttgtaaatatgatgctctgcgaacgtgaattaacaattgaaagagccttaaagaatgaattcgaggggaatatggcaaatgtagaatattttgctgaagggctcggtgaattatgtcattggaaagagatgatgcttcgtcagagacacaacgggtcgctctttgattcaccagccactactgcagctgccttgatttaccatcagtacgatgagaaatgctttgggtacttgaactcaatcttgaaactgcacgataattgggtccccactatttgccctacaaagatacattcaaatctcttcttagttgatgcccttcaaaatcttggagtagatcggtattttaaaacagaagtcaaaagagtactagatgaaatatacaggctttggctagaaaagaatgaagaaattttttcagacgttgctcattgtgccatggcgtttcgacttttacggatgaataactatgaagtttcctcagaagaacttgaaggatttgtcgaccaagaacatttctttacaacatcaagtgggaaacttatgaatcacgttgcaattctcgaacttcaccgagcttcacaggtggctattcatgaaaggaaagatcacattttagataaaataagtacttggacaaggaattttatggagcaaaaactcttggacaagcacatccctgataggtcaaagaaggagatggaatttgctatgaggaaattttatggcacatttgatcgagtggaaactagacgttacatcgagtcatacaaaatggacagttttaagatcttaaaagcggcttacaggtcttccggtattaacaacatagacttgctaaagttctcagaacacgattttaacttgtgccaaacccgacacaaagaagaacttcaacagatgaaaaggtggttcacagattgcaaactcgaacaagtaggattatcacaacagtacttatacactagttacttcataattgctgccatactctttgaacctgaatatgctgatgctcgtctagcatatgcaaagtacgccataataataacagcggtggatgatttcttcgattgttttatttgcaaagaagaactgcaaaacatcatcgaattagtagagagatgggagggatactcaaccgtcggattccgttcagagagggttagaattttctttttggcactttacaaaatggtagaggaaattgcggcaaaggcggaaactaagcaaggtcgatgtgtcaaagatcaccttattaacttgtggattgatatgttgaagtgtatgctggtggaattggacctttggaaaattaaatcaactaccccaagcatagaggagtacttgtctgttgcatgtgtaactattggtgttccatgttttgttctcacatcactatatcttcttggaccaaaactgtccaaggatgtcatagaaagttctgaggtcagtgccttatgcaattgtacagctgctgtggcccgattgattaatgatatacacagttacaagagagaacaagcagaaagttcaacaaatatggtatcaatattaataacacaaagtcagggaactatctctgaagaagaggctataagacagataaaggaaatgatggaaagtaagagaagagagttgctagggatggttctacaaaataaagaaagccaattgccacaagtgtgcaaggatcttttttggacgacaatcaacgcagcttattctatacatacacatggcgatgggtatcgcttcccagaggaattcaagaaccatatcaacgatgtaatttacaaaccactcaatcaatattccccataa

SEQ ID NO: 44 - DNA, 1792 bp, Nicotiana sp.
  source 1..1792: mol_type = genomic DNA
atgtcggtctttgcggttatttcattctttctacttctgttttttcttttcaaatcatatttgccctcatcgaaaacaaagaaaaattctccaccatctccttcaaagcttccgttaatcggtcacttccacaaactaggcttacaacctcaccgttctctacaaaaactatcaaatgaacatggtcccatgatgatgcttcaattcggtagcgtacctgtgcttatcgcttcatcagctgaagctgcttccgaaatcatgaaaacccaagatttgtcttttgcaaacaaacccatttcaaccattcctagcaagcttttcttcggcccaaaggacgttgccttcaccccatatggggattactggaggaatgccagaagcatttgcatgcttcagcttttgaacaacaaaagagtccagtcttttcgaaagataagggaagaagagacttctcttcttctccagaggattagggaatcgccaaattcagaagtcgatttaacggagctgttcgtttccatgactaacgacatagtttgcagggtggccttaggaaggaagtattgtgatggggaagaagggaggaaattcaagtctttgctgttagagtttgtggaattgttgggagtttttaacattggagattacatgccgtggcttgcatggatgaatcgtttcaatggtttgaatgccaaagtggataaagtggcgaaagagtttgatgcatttttggaggatgtgattgaggaacacggaggaaataagaaatcagacactgaagctgaaggggcagacttcgtggatatattattgcaggttcacaaagaaaacaaggctggttttcaagtcgaaatggatgcaatcaaagctattatcatggtatgtgtatatatatatcactggcattcaactttaaactttcattttactcattttactcttgatgagaagcatttataatcacataaatgtcatgtccccacaaaacatttaccccttaaacttctaagaacacaactttcaaaaaataaaatttttcataaatttcgtgtcgagtcaaactaagtcatgatctaatttgaaatggaaggagcatataattaccctctaaacgaacaaattaaactgaaagagaaaactatatattgtatttgatttacatgtgctaattaattgtttgatgcaggatatgtttgctgcgggaacagatacaacttccacgcttctagagtggacaatgaacgagctcttaagaaatccaaaaacattgaataagttgagagatgaggtgagacaagtgactcaagggaagacagaggtaacagaggatgacttagagaaaatgccgtatttaagagcagcagttaaggagagttccaggctacactctccagtgccacttctacctcgagaagcaattaaggatgcaaaggttttgggctacgatatagctgcagggactcaagtcctcgtttgtccatgggcaatctcaagagatccaaacctttgggaaaatccagaggagtttcaacctgaaagattcttggatacttccatagattacaaaggcttacatttcgagttaattccattcggtgcaggtcggaggggttgccctggcatcacatttgctaagtttgtgaatgagctagcattggcaagattaatgttccattttgatttctcgctaccaaaaggagttaagcatgaggatttggacgtggaggaagctgctggaattactgttagaaggaagttcccccttttagccgtcgccactccatgctcgtga

SEQ ID NO: 45 - DNA, 3575 bp, Nicotiana sp.
  source 1..3575: mol_type = genomic DNA
atggcagaaaaaatcaccagccacgagaacacaaggtatttacaaaccttgttatgtctattaagttttatgtactgatagtaaaaaattatttacggaatcagtacggttattaggtaattataggtaaatctctactataagcatagtagtaatatttaagttgtatgatcgatcaggtatgcagtggtgacagggggaaataaaggaataggatatgaaacatgcaggcaactagcaaaggaaggaatagtggtagtgttgacagcaagggatgaaaggagaggaattgaagctctcgaaaagctcaaggaagagtactcaagcaataaaactgatgatgatcagattttatttcatcaacttgatgttatggatccagctagtatttcttctcttgtggacttcatcaaaactaaatttggaaagctcgatattctggtactattcactactattatatttatcaccccatcacttaaatctagggctaagttggttgggttagtgatttcccacgtgagagaccttagatccatttttttcccagcagccttctcctcgcgctgggatgtctagtgcaatttatattttctatgtgatttgcgaactattgcacagtaaataggggcacacatccgaagggtagcggctgctgaacctgagaattttcccccaaaatatcgtttagaaaaaaacaagaagtcatctattattacttttttccagatccattcttcctgtttcaatggatgaagtattaactaattaaatgagccatttaaggaaagataaagggtgattaaataattactagtattaaatgactctcttaaggggtgtgtaaaacccataaaaacaataacatgtgctggagggaacagtaattttattgtcagtatggtaaagcttcgtatatttttctatttacttgtactgaattcttgatgttttcatgttagagatatatgaagtttaaccctgcaaaatgttatgcatgtggtaaatattattacagaactaggatattttggctgactggcttatccctcgttagaaatgaatactgcaaagctccatatctatgtagttaatcaagtcgcaatgcatctataccgtagagtgcaatttatcttactcctatatactctactcgcttcttcgtgtacgtaataagtaattttttagctttttagcatatctcactcttagaaaaaagtattttgattaaattaccttaattaaacttgcatattgttaacttgacatcttgaaataagaaaataaggaattttttatttaaaaataacattaatacttgttgataatatgaaaaccaaaataaaaagatttgtagctctattcagtgatcagtagccaaagaaagaaaggtcgaggaggtgaaaaaggtatagggtccttgagatctacaaaagttggtgaacagtgacatgcaatctaatttatttaggattaacatttcttttccaagtgaggaagcccatcagttgttagatggccccttgagatctacaaaatagattctttttgtgcaaaagataatattcattaattaaatgtatgcgtaccgagagagtggtgcgtagcttgcaccatattacaaagtactaaactattacttgtatcttctacagaataggtaacttatggttatcataggaattacttgtactttttttatgatagtgtaaatatttctttattatcgatgtataattataaactcgctcacaaattagtgaaataagatataacttatgggtacagagtcgtacttaacattgaaattgtaagatatacgattaatattttattaaatttcctacatcttttttcagtgctataggttgtcttagcaaggtagtcatgagggacaaaatattttagtgctaattagttttggttttaatctcttggggtcgtttggtagagaacaagttatcccgagattataatgatgaaaattataatacagaaattaaataatgatgagattatttagtgaatatatttttattggactaaaaatggggtacatgggtacatatttcatatatgctttacttagaaataagaaattatgaatgcttcaatttaatttgcaggttaacaacgcagggattggtggattaatggtagaaggagatgttgttataataaaagatttaatagaaggagacttcgtaaccatttctgctgaaaatggggtaagatatatagccaatgctaatatgttatcctaaattaattagaaacagtcaatggtgatctctttgataattttcagttattttatgtcacaaatacattcaaagaaatagtaacaaattgaagtaattattatgcaggaagaggatggtattaagaaatcaattgaaggtattgagcgtattgttacagattatgagttgacaaaacaatgcctggagacaaacttctatggtgcaaaaagaatgattgaagcatttattcccctccttcagctctctaactccccaagaattgttaatgtcgcttctttcttggggaagttaaaggtaatgaaatcataactgaatactatttcataatatatggaccctctgaattatgttgagtttaatttctacatacttataatagtggtagcaaatggcctgtttgggctgatcaagatagattgactcattgcccaattctgcctaaatggttaagataggctaaacacaacccatccaaattgacccaaatctttgcaatattttcaacttttaaacaagcattataaaatattcttacatatataataatatttttcgtaaatatcattttggaatggtttacgatgatgattttttttttctttttttcctgttacttgtcttatgttttgaaaaaatgttaatttatgcctaatgatttatttcctcaatttcgatttaaaactttatttgatagttccttatcgtataatactaacaaaaatcagaataataaaaaagtagtaaatcatgggttgatctgggctactgaaaataaatgggcttagttgggttgtgcccaaatttagcccatcactaaagtagcttatgtccaacccactaaaacctaacggattgggcgggtcaaatgggtttgtgcaacttttgccaccctaagtgatagtgtactcctaaccgttttccgctattctacagctattgtgcaaccaatgggctataggaatgctaagtgatgctaaaagcctgagagaagaaagggtggatgaagtgttgaatgaatttataaaagattttaaagagaaatcaatagaagccaaaggatggccaacttatttctcagcttacaaagtctcgaaagcatccctgattgcttacacaagggttttagctacgaaatatccaaattttcggataaattctgtgtgtcctggattttgcaaaacagacgtgaactgcaatactgggagcttaactgctgaagaaggtgctgaaagcttggtgaagcttgctttggtgccaaatgatggaccctctggtctcttcttttatagaaaggaggtcacctctttttga

SEQ ID NO: 46 - DNA, 4497 bp, Nicotiana sp.
  source 1..4497: mol_type = genomic DNA
atgagtcaatcaatttctccattaatctgttctcactttgcgaaatttcagtcgaatatttggagatgcaatacttctcaactcagagttatacactcatcatatgcctcttttggagggagaagaaaagagagagtaagaagaatgaatcgagcaatggatctttcttcaagctctcgtcatttggcagattttccctcaacaatttggggtgaccattttctctcctacaattctgaaataacagtaagtcacaatcccatgcttatattctaccgtcaaacttctctataacaactccatttgttgcgatcgttttttattgttatagtgaagtgttgttatagagattatatattataatataacatagtaatcggttccgagaaaacttgattctttagtgaatgactgttatatggggatgttatagagaggtctgactgtgttacctaaatgaataatgaagatgttataattttattagagatggatgaagcatttaaacatgaaagattcaagctttaagattggtctttatcgtacttttgaaattatgagtttggaatttaatacttcctccgtctcaaaaaagaatgaatcttttactatatggggtcaaataagattttctttggccatattttttgcaaatgcatttcaaatattttgaatttttaattgttgtgacttacaataccttttatgcagtttctaaatgtgtaaaatatatttcaaaacatttaaagaatatatgttcatcacattaaaaaatattgaacttgactctcatgctccgacaaggtttaaataaattgaaacgaatggaatattactattttgtaaaaacttaggtaggttttcactatatatctaataatgtattcaaaactcataaataatgatcaaaaggagccttgtgaccgggaggtcacgggttcgagccgtggaaacaacctcttgcaaaaatgtaaggtaagactgcatacaatagactcttgtggtccgccccttctccggaccccgcacatagcggaagcttattgcactaggctgccctttttagtgattaacatatgtggatctaggatttaaattttttgggttcaacctttaaggatctgttacaattttagtagaatgttacacataaatttgtgctccgtgaatgtattgagtcagatgaacctggtattatacatgcggatacgctcctgatgctcaatttctgcctgcaggaaattactacccaagagaaaaatgaacatgaaatgctaaaagaaatagttcggaaaatgttggtagaaactccagataatagtacacaaaaactagtcttgattgacacaattcaaagattgggattagcatatcatttcaatgatgagattgaaaactccattcaaaacatctttaatttgtctcaaaatagtgaagatgacgatgaacacaacctttatgttgctgctcttcgttttcgacttgcgaggcaacaaggatattacatgtcttcaggtaccttacatttctgccctttcccgcacagcttcattttttttcgttgttaaaagacagttcggcgcataaaatatctcatgtatacgcagggtcaggacgaaccgcccccaaggggtgtaaagtatgcaacttaccctaatactaaatatctcgtgtatacacagggtcaggacaagtcgcacccaaggggtgtaatgtagacaacttatcctaatgctattagtaactgattttatggctcgaacacataaattataggtcacacagtaacaactttaccgttgctcaaagactcgccttcctctttttttagttatcgcaccttatttgtgcagagaatagcaagtttcgagatctgcttctatatagaagacttctgtattatacttttttattttgtccttctgcttaaaaatagtaaaaaactatagtgtggaaattgtaaatttcttaactagctgtgaaatcaaatagttattataggaatattatttaagactccacttatggaaaaccactgggttgttgttgttattgtcaataataacttggggtacgatttacttctttttccatggcttgtccacgactatatttctattaacaatgttgtgactatgctttctttgagtcgagggtctattgataacaggctctcgatctttacaaggtaaaagtaatgtctgcgtacacactctactccgcagactccacttgtaggatttcactgaatattttttgttgttgttgttgttgtaataacttagggtttaatttcttgatgctaatgaaattcatttctttcaaaatataaacatggtgttcaaccagatgtgttcaagcaattcactaaccatgacggaaaattcaaggaaaatcatactaatgatgttcaaggattattgagtttgtatgaagcagcacatatgagagtgcacgacgaggaaattctagaagaagctcttatctttaccacgactcatctcgagtccgtgatcccgaatttgagcaactcgcttaaggtacaagttactgaagccttaagccatcctattcgcaaagctataccaagggtgggagcaaggaaatacatacacatatatgaaaacattggaacacataatgatttacttttgaaatttgcaaagttggacttcaacatgttacaaaagcttcatcgaaaagagcttaacgagctaacaaggtacatctactattcttgtcatcttcataattatggtacaatcagacctctctataaaatacatcctttataacaacagttcactataacggtcaagttttctttaaaatcaatgttttatgttaccaaattattttgaaagaaatgtgactattatagagaggtttgactgtaactcgcgctaattaataacacctaaagtttaagtatgttaatgctgttatgatatctatagctggtggaaagatttggatcgtgcaaacaaatttccatatgcaaaggacagattagtagaagcttacttttggacggtgggaatatattttgaacctcaatatagtcgttcaagaagtttggtaacaaaagtagtcaaaatgaactccattattgatgacacttatgatgcttatgcaacttttgatgagcttgtgcttttcacggatgcgatccaaaggtaaaattatatataataaaatctttctataacaacgtcatttattctgatatttttttaagatgctatagtgaagtattgttatatagaaatatattatgacaacttagactttgcagatgggacgaaggtgccatggatttattaccgacatatctgagacctatttatcaaggccttctcgacgttttcaatgaaatggaagaagtattggccaaagaaggtaaagcagatcacatctactatgcgaaaaaagaggtaatccttgattaagttacattaattactacttaatagttaattaagtaaaccaagttgtagggaagaatcgcaattttgaactattagtacattttctgttacttttttagatgaaaaaggtggcggaagtctattttaaggaagctgaatggttgaatgctaactacattccaaaatgcgaggagtatatgaaaaatggacttgtaagctctaccggtccgatgtatggaataatttctttggttgttatggaggaaattataacaaaagaggcttttgaatggttgacaaatgaacctttgattcttcgagctgcatcaacaatttgtagattaatggatgatatggctgatcatgaagtaagtataacaatataattttcattttatataacaatagccatcgtaattcgcgaattttgtccctaaatacaatacaaaaacaactacaataacaaaaacaacatatccaatatattcctacagtacgggtctaggaagagagatgtgtacgcagatcttaccctaccttatagaggtagaaatgttgttcccgatagaccctcgactcaaaaaaagcatttctcagtctgatttcgagtctaggtggcacttttgcatgataaaataaatagacatgcttgataaattacaacttcaatgatcacatttacttaaactgaattatggaactttagaaacggctgattaaaatggtaaaatattgtataatattaatgaagaaattgaaatatattatgttgtaggttgaacaacaaagaggacatgttgcttcatttgttgagtgctacatgaaagaatatggagtttcaaagcaagaagcatatgttgagatgcggaaaaaaatcacaaatgcgtggaaagatataaataaggaactcttgcgccctactgcagtaccaatgtttatcctcgaacgatctttaaatttttcaagattggccgatacatttttgaaagatgatgatggatacacaaatcccaaatccaaagttaaagacttgattgcttcgttgtttgtcgaatctgtcgacatatga

SEQ ID NO: 47 - DNA, 1098 bp, Nicotiana tabacum; identical to SEQ ID NO: 42
  source 1..1098: mol_type = genomic DNA

SEQ ID NO: 48 - DNA, 9552 bp, Nicotiana sp.
  source 1..9552: mol_type = genomic DNA
atgatacttggactgagaagcaaaatcataccacttcctgatcataagttgggaaatatcaaattaggttcagtaaccagtaagacacataatttttattactatacgttacaagaagataagtatcattcaattcttttataactaatttcagtctcttttttgataaaatttctgtgtctccttgttcgaagaagatgcaatttgccacagaccatgtagagtaagatgcagccacagtactgcttcatcaatggtatgctatttctttttgcatacaaatttagccaactggagtaactgagacagaattttaaaaagtggcctagaaaaggccatttgtgtaaatgcccttaataaagcaatggtggttgcaggaagaggcaaaggagagaataagggaaacatttggaaaaatagagctatctccttcttcctatgacacagcatgggtagctatggtcccttcaagatattctatgaaccaaccatgttttcctcagtgcttagattggattcttgaaaatcaaagagaagatggatcttggggcctaaatcctagccatccattgcttgtaaaagactccctttcttccactctagcatctttgcttgcccttcgcaaatggagaattggagataaccaagtccaaagaggtaaatgacaccgagtttagaagaacgatttctttgaaatttgtggttgtaaatatgcaatgatatttttttggttataaaacaaaaaatttaaagttaaattttatccaaatatataaaggtgtcactcattttaaaactgacctgaagaaattacatcatttgaattaattttttctgtgtagtgtcagaatattaaaattttaattgtttacaaatcagttatatatcttagtgtagcttagctccaaaggttaaataatatatgaataaaagatatatcttgtaagctaattgatctcccattagcaagcgttcagggtggtcctttgtaacaggtcacagaccaacagtaagaagtttgcttgtctgtatcttgtttgaagcggttttgattttatgtctcccatctgtgtactaattaatttatttttactatataaggtaggagataatgccgaaccccatcatcatgggccagcatagcgcagggtgaatggcttcaattaaaaaaaattaaccttagtagagaaaaattaaagagaaaaacagaatgtgtgtatatcacttaaccatatggttaaataataatataatatatgaataaaagatgcatgcattcattagtttgtgtaaacactaagcagaataaaatgtttagttaccatcttgcatgttataattgtcaagtatctagacgatgtgcaaaaacaagtaatgctattgacatggttcaagaaagagtgagcaaaggagtttgcattttgtagtattattaattacttgaatagttccttcatttttcttttaggccttggctttattgaaacgcatggttgggcagtcgataacaaggatcagatttcacctttaggatttgaaattatatttccctgcatgatcaactatgcagagaaacttaatttggatctacctttggatcctaaccttgtaaatatgatgctctgcgaacgtgaattaacaattgaaaggtatgcttctttattaaatggtcattttacatattatggaaattattagttttgcagccttgcgcaaaaactcctaatgtataaaacaattataggggaagttcaacaatattagtcgtctgaagctaaatcttaataaaaggcctaagcgtaatctcattcgattcatatattaataagaaaaaggacatattttttatatctatagatcaataatcaaacatgacaaagagaaatatcaaaaaaactttgttgaaattggaaagataaatgactagaaataggaaaaacgaatggatatatgcatgaaaggaaatatcatagagggtaggagaggtaaatgtactactagtagttaataatcttttggaagaagtttacacatctactcatccgtccaaacagtaaaaagctaacataaattttgaaacaacttataagtgatagttatttcctttgtttcaatttatgcggcaacattttgtttttggtccattttataaaaaatagtgactttctaaatttgagaagattaaacttctcatttttattcttaatgagaaatttttatatatatcctcacaaatgtctatgatttttttatcataaatttcaaaagtatcctttatttcaaactccatagtaattcaaattttaccacataaagtctcccatggtggtatataataactgtaattttttgcctgtaattattgagaccaaaagtactaacttcatttgtcttgcctttaatcgatcacacgaacaattatacgacggtgtttgactaggtactgaacttaaggaagattgaatgacttttgccacatgtctaaagtgcgctagacttacacatgacatagcatttatttgattataaaagtatctcattaaggataaattaaaagtttacaaaaagttatcaaataaagaatgggattcttcttttgtgaaaagacattaaaaaaaacatgtcatataaaatggaacaaaggaatatagaatttgccctctccacgaatttgtatgcagatttgtaaattgacttataaaactcctaactttatggagtaagtctcttgggaaaatgtcattctgaagaatgataaagcaaaaaaaaatagtttaaaatttgtagagtgcaaaacttcatttttaatttaaacatgactgcaacactcaccccttccaaaaatagaaagaggtcattctttttttgtacagactaaaaagaaaataggttcacataaattgaaacgaaaggagtactagattttgatactctagcttcaactcatcaatttgttgggccattttttaaccaatagagccttaaagaatgaattcgaggggaatatggcaaatgtagaatattttgctgaagggctcggtgaattatgtcattggaaagagatgatgcttcgtcagagacacaacgggtcgctctttgattcaccagccactactgcagctgccttgatttaccatcagtacgatgagaaatgctttgggtacttgaactcaatcttgaaactgcacgataattggggtatgcactactaagatcatctcaatacttccccattatctgcctaaaggaaaatacttttcatatctcttcttagaattcgttggaattaagttttaattatgttagtatgtttactatatgttcgatatgtttgataggagactaatagttttagggtactagaaaatataaagtacttctactgataagaaatttatgttagtatgataatttagcgcttgtgacgcatggtgacatccttaccgcttctccccacaagtacaaatgtacatctacgaacaactcacatattgactcaaatcatgctttagagaaaatcacaagaagagtaagtctaataaacttacctctcaagtccaaactctgacatagtactacgatgaaccaatttatttaaaaagctcggacgtcatcaacaaagggatatctacatatatgtggaaaatggcagaattctgaccgccaaaacagtcggaattcacgaatttcctactactttccgaccaaaattggtcggtcgaaaaacagttggtcggaaaataattttcgatcaaatccgttagtgatttccgaccaaagtagtcggaacgttcgaaaggttagacgaccaaatattttcaaatttccgaccaaagtggtcggaatttaccgaccgaatcagtcgtaataatataaataaaataattatttatttaaaattaaataatataaatatataatttccgaccgaatcggtcggaaattaatatttaaaaaatataatttccgaccgaatcggtcggaaattaatatttaaaaaatatattttatttaatttccgacgaaatccgtcgaaaactttttaatttttttttaaaatttccgaccgcatcggtcgaaaatctgggaatttttggcaaaatgtgggcagctgtccgactgtttccgtcggaaatcagtcggaattttctgtaaaactgagcagcattctgcccaatttggagctacaccacgtgtcaaactattacaatataataacgccaacaaccaatcaaccattaacacaatctaaaccaacaataccaatcattaacataatctaaactaacaataccaactattaaaataatctaaaccaaccattaacacaacctaaactaacaataccaaccattaaacctaacaattttggacataaaaatatccaaatagtagcccacattcaagtttaaaaataaaacataaagttgtctctcacaatcaagtttaccaatcaaccaaaataccacacctaaactactacacatcagattcagtttgttcatcctcatcatcagatagatcatgcccatgttttctcatgaatttctggcagtggagggtcgtcgacggagctgcagcgaggcggcatcgccggtgagtggcattggtgatggcagcggcgggttgggtgggggggagaggggaggtttgagtgtgagagagagaagagagaaggaagaaaacagggaaagaaaaaagaaaggggtcaggggggttttaaaagaaaataggtcaaatagtcgaacctcgttttaataaaaaattccgaccgaattagtcgataacagttatgcggtatttttcgccaaaatttacgacggatatagacggaatatcagccgtaattattattaaaaattgaaaaatatatttttatgcaatttccgaccgatttcgtcagaaatttgcgactactttttccggtcggaatatttcggtcggaaattggccgttttttagtagtgatatatgagttcatatatcaataacgcttcataacaataccaactaagtaagtagcacttaggtccaacttcacatgactttcacaaaaattcttatttcaccatttgatatcaatccttgaattttaactccaattcatctattaaatgtgttatggaatctagccatttcattagaagttcaaaacgatatcgttcaaataagtccaacactattcatcttccctattaataaactttcagtgtcaaagctatgataggagggttcatctattccacccttctaactactttttcagctcataaataatattaaaacactcacttatacttcataaataaggttcatgtagtaaaaattatctttgtaagaaaatcctagaaaatctctcttttggtgtttttggttgatttaataaaatttagcaaactaaaggaagtttcaatgttaatactagaattaattaactttaatgagcacaaaacatcgataaaaacttgccttgtatgacccaaatgaacccttggtcaaaatcctctcgatcctaaatcatctaactaataatgagttgtacacctgagtatttaaagacactgtaaactaacttctaactaataactaactaacagctaattacaaagatcaataactaacactaatttatgaaccttctagaagtgtactcctttaaataatacactgtacaacctgtctaagctacatatttcagcattactaatataatctttttcgttattcttatagtccccactatttgccctacaaagatacattcaaatctcttcttagttgatgcccttcaaaatcttggagtagatcggtattttaaaacagaagtcaaaagagtactagatgaaatatacaggtaatttgaaatgatcttcatggtttatacaatttatctttttaaaacttttattaatattaatgctgaattatgaataggctttggctagaaaagaatgaagaaattttttcagacgttgctcattgtgccatggcgtttcgacttttacggatgaataactatgaagtttcctcaggtttgtaactaaaaactcagaaaattaaatttttacctatgcaccaataatataaggggtgtcttgtcgtaattggtaaagttgtttgtcgtaattggtaaagttgttggtcacaagttcaggccgtgcaaatagcctcttgcaaaaatgcagggtaaggctgcttagaatatacccttatggttcgacccttccccggacctcgcgcatagcgggagcttaatgcaccgggctgtccttttacttatgcaccaataaattgttactttctttctgcagaagaacttgaaggatttgtcgaccaagaacatttctttacaacatcaagtgggaaacttatgaatcacgttgcaattctcgaacttcaccgagcttcacaggtggctattcatgaaaggaaagatcacattttagataaaataagtacttggacaaggaattttatggagcaaaaactcttggacaagcacatccctgataggtcaaagaaggaggtatcacctgtatataaattaggccattcaaaaaacattctttgtaattttcaattttatgatattccattactagcgagtcaattaaatatcagatggaatttgctatgaggaaattttatggcacatttgatcgagtggaaactagacgttacatcgagtcatacaaaatggacagttttaagatcttaaaagcggcttacaggtataattctgacactattactataagattttagataatatgtaccgtcagcgggttacatttgccaattattgcaggtaatctatattgttttcaagattataaatctcacattttaaggaggcttacctgtaaatattatttgagtgacggttaaaaaaactttacattaatgatgtatgtaacgtaaactcttactaaaagtagtatatatgttttgtttattcttattattctcttctgcatttaaacgtgtggacaggggcagatttataggagagattatggggcacgcaaacccatgatctcctcaaaattagatattttatgtacatattttttaaaattggtataatattaactattggcatccatgcttctatttaatatatttttcctctttttattttagtaatacattcatgtactgaaaatcctagatccacatctgtatatgggcggatcagagttacggttgtagcaatgcccaccgccttcaacttaaacaacatatgtcagtgaacattggtatgctaacgcttaatatgggtagcaagaaaaaatacaagctactttaaataaaatgtaatggtacattttctatcgaagttggaagtccttaaaaattggtcatattattatgtggggtctctctctttctctcttttaggtcttccggtattaacaacatagacttgctaaagttctcagaacacgattttaacttgtgccaaacccgacacaaagaagaacttcaacagatgaaaaggtatctacactggttagcttgactagaccttgtcattcttaaacaacttcattttgttgataattatatatatatatatatatatatatatatatatgaatgtaggtggttcacagattgcaaactcgaacaagtaggattatcacaacagtacttatacactagttacttcataattgctgccatactctttgaacctgaatatgctgatgctcgtctagcatatgcaaagtacgccataataataacagcggtggatgatttcttcgattgttttatttgcaaagaagaactgcaaaacatcatcgaattagtagagaggtattgttatctcttttcttataatatatcaacaaaatcctttgacctatgttgttcaactcacattttttagaggcacttgagtttatgataaagaatgttacacttgtccctcatcaattttgtacgggcttttaaaagggttcaaatgatcctaggctacttcattcatagctttatggttttggatttatactaatattctttcacatggtatcagagccaaaactttctttttttcattcagaccattaagccactaacattgctatggcctaacttatcaattatcatgttgagcctaatctacttaactcttgaagatcacaatttgttgagactcttctctttctacgttaaaaataataaccaaaacagaaacaaaccgtagaaccgggctgtgttgcaagagagtattagagaatgttacacttgtcccttagatactccacccgattaccttgaaattttggattagatactcatactctttcacatattatgttaacactttcactatagatgggagggatactcaaccgtcggattccgttcagagagggttagaattttctttttggcactttacaaaatggtagaggaaattgcggcaaaggcggaaactaagcaaggtcgatgtgtcaaagatcaccttattaacttggtatgaagaagtacattaattattatatattctctaaatcaatttttttttcatgcttatatttctttcttttcgattgactagaaatatatcttgttatagtggattgatatgttgaagtgtatgctggtggaattggacctttggaaaattaaatcaactaccccaagcatagaggagtacttgtctgttgcatgtgtaactattggtgttccatgttttgttctcacatcactatatcttcttggaccaaaactgtccaaggatgtcatagaaagttctgaggtcagtgccttatgcaattgtacagctgctgtggcccgattgattaatgatatacacagttacaaggtaatattattttcttcgttagaagaatattttatcaaatgttggctattgatttttgacaatcttttttcaatatattgaactgagataaaattttacataaaatattcttttttcgaaacactaacttcttcaaatcaaattttattccaaaaattactaaaatttacatttgaaagtgagaagtgacacaaaaacaaagccttgtccggctagattgatatttgataaattaatataatataatgtgctgctggttcattttcttcttattaagctcgtctcactaagttttcgcgctagaaggatcagaccacgttgaatttattttgtacgcttttttttcaagtagacatacaagtgtataattataacattaatccttattatttcgtgtgttcttgcagagagaacaagcagaaagttcaacaaatatggtatcaatattaataacacaaagtcagggaactatctctgaagaagaggctataagacagataaaggaaatgatggaaagtaagagaagagagttgctagggatggttctacaaaataaagaaagccaattgccacaagtgtgcaaggatcttttttggacgacaatcaacgcagcttattctatacatacacatggcgatgggtatcgcttcccagaggaattcaagaaccatatcaacgatgtaatttacaaaccactcaatcaatattccccataa

SEQ ID NO: 49 - protein, 494 aa, Nicotiana tabacum
MSVFAVISFFLLLFFLFKSYLPSSKTKKNSPPSPSKLPLIGHFHKLGLQPHRSLQKLSNEHGPMMMLQFGSVPVLIASSAEAASEIMKTQDLSFANKPISTIPSKLFFGPKDVAFTPYGDYWRNARSICMLQLLNNKRVQSFRKIREEETSLLLQRIRESPNSEVDLTELFVSMTNDIVCRVALGRKYCDGEEGRKFKSLLLEFVELLGVFNIGDYMPWLAWMNRFNGLNAKVDKVAKEFDAFLEDVIEEHGGNKKSDTEAEGADFVDILLQVHKENKAGFQVEMDAIKAIIMDMFAAGTDTTSTLLEWTMNELLRNPKTLNKLRDEVRQVTQGKTEVTEDDLEKMPYLRAAVKESSRLHSPVPLLPREAIKDAKVLGYDIAAGTQVLVCPWAISRDPNLWENPEEFQPERFLDTSIDYKGLHFELIPFGAGRRGCPGITFAKFVNELALARLMFHFDFSLPKGVKHEDLDVEEAAGITVRRKFPLLAVATPCS

SEQ ID NO: 50 - protein, 321 aa, Nicotiana tabacum
MAEKITSHENTRYAVVTGGNKGIGYETCRQLAKEGIVVVLTARDERRGIEALEKLKEEYSSNKTDDDQILFHQLDVMDPASISSLVDFIKTKFGKLDILVNNAGIGGLMVEGDVVIIKDLIEGDFVTISAENGEEDGIKKSIEGIERIVTDYELTKQCLETNFYGAKRMIEAFIPLLQLSNSPRIVNVASFLGKLKLLCNQWAIGMLSDAKSLREERVDEVLNEFIKDFKEKSIEAKGWPTYFSAYKVSKASLIAYTRVLATKYPNFRINSVCPGFCKTDVNCNTGSLTAEEGAESLVKLALVPNDGPSGLFFYRKEVTSF

SEQ ID NO: 51 - protein, 598 aa, Nicotiana sylvestris
MSQSISPLICSHFAKFQSNIWRCNTSQLRVIHSSYASFGGRRKERVRRMNRAMDLSSSSRHLADFPSTIWGDHFLSYNSEITEITTQEKNEHEMLKEIVRKMLVETPDNSTQKLVLIDTIQRLGLAYHFNDEIENSIQNIFNLSQNSEDDDEHNLYVAALRFRLARQQGYYMSSDVFKQFTNHDGKFKENHTNDVQGLLSLYEAAHMRVHDEEILEEALIFTTTHLESVIPNLSNSLKVQVTEALSHPIRKAIPRVGARKYIHIYENIGTHNDLLLKFAKLDFNMLQKLHRKELNELTSWWKDLDRANKFPYAKDRLVEAYFWTVGIYFEPQYSRSRSLVTKVVKMNSIIDDTYDAYATFDELVLFTDAIQRWDEGAMDLLPTYLRPIYQGLLDVFNEMEEVLAKEGKADHIYYAKKEMKKVAEVYFKEAEWLNANYIPKCEEYMKNGLVSSTGPMYGIISLVVMEEIITKEAFEWLTNEPLILRAASTICRLMDDMADHEVEQQRGHVASFVECYMKEYGVSKQEAYVEMRKKITNAWKDINKELLRPTAVPMFILERSLNFSRLADTFLKDDDGYTNPKSKVKDLIASLFVESVDI

SEQ ID NO: 52 - protein, 792 aa, Nicotiana tabacum
MVLGLRSKIIPLPDHKLGNIKLGSVTNAICHRPCRVRCSHSTASSMEEAKERIRETFGKIELSPSSYDTAWVAMVPSRYSMNQPCFPQCLDWILENQREDGSWGLNPSHPLLVKDSLSSTLASLLALRKWRIGDNQVQRGLGFIETHGWAVDNKDQISPLGFEIIFPCMINYAEKLNLDLPLDPNLVNMMLCERELTIERALKNEFEGNMANVEYFAEGLGELCHWKEMMLRQRHNGSLFDSPATTAAALIYHQYDEKCFGYLNSILKLHDNWVPTICPTKIHSNLFLVDALQNLGVDRYFKTEVKRVLDEIYRLWLEKNEEIFSDVAHCAMAFRLLRMNNYEVSSEELEGFVDQEHFFTTSSGKLMNHVAILELHRASQVAIHERKDHILDKISTWTRNFMEQKLLDKHIPDRSKKEMEFAMRKFYGTFDRVETRRYIESYKMDSFKILKAAYRSSGINNIDLLKFSEHDFNLCQTRHKEELQQMKRWFTDCKLEQVGLSQQYLYTSYFIIAAILFEPEYADARLAYAKYAIIITAVDDFFDCFICKEELQNIIELVERWEGYSTVGFRSERVRIFFLALYKMVEEIAAKAETKQGRCVKDHLINLWIDMLKCMLVELDLWKIKSTTPSIEEYLSVACVTIGVPCFVLTSLYLLGPKLSKDVIESSEVSALCNCTAAVARLINDIHSYKREQAESSTNMVSILITQSQGTISEEEAIRQIKEMMESKRRELLGMVLQNKESQLPQVCKDLFWTTINAAYSIHTHGDGYRFPEEFKNHINDVIYKPLNQYSP

SEQ ID NO: 53 - protein, 365 aa, Nicotiana sp.
MAFLATISGHENMLLSNTLNNNFIFSGKPPQRHSYSFLPKKIQARSVANSSKTFQVKEEEFSSKTEKFILPKFDFEEYMKMKAIKVNKALDDAIPMQEPIKIHEAMRYSLLAGGKRVRPILCMASCEVVGGDESLAIPAACSVEMIHTMSLIHDDLPCMDNDDLRRGKPTSHKAFGEDTAVLTGDALLSLAFEHVASKTKDVTPQRVVQAVGELGSAVGSKGLVAGQIVDIASEGKQVSLTELEYIHNHKTGKLLEAAVVCGAIIGGGNEIEVERMRNYARCLGLLFQVVDDILDVTKSSEELGKTAGKDLVTDKATYPKLMGLEKARELAGELVAKAMDELSYFDAAKAAPLYHFANYIAHRQN

SEQ ID NO: 54 - DNA, 354 bp, Nicotiana tabacum
  source 1..354: mol_type = genomic DNA
atggctaggctattctatactatgatcattctcttgcttgctttagcaacatcagcaattgctgaaccttcttgtaaaactgttgcaaaagggatagtaccttgtgtgtcttatattagagggaaacatcataaatcggacaagccatcaaatatgtgctgcaaaggactgaatgacatagccaatgtgataaaaaatggcaaggatcgtgtagctgtttgcaagtgtataaagatggcactttcacgtattcattatgatcccactcgtatcacacttgcttcacaacagtgtcatacgccttcatctctgccttccgttggccaaaacactaattgtgcaagggcgatctga

SEQ ID NO: 55 - DNA, 351 bp, Nicotiana tabacum
  source 1..351: mol_type = genomic DNA
atggcaagagagaaatctgtaatgtgtatggtgatggttttgggggttgccttaattattcagggcactggtgctgctgaagaatgtagcacagtgacagcactggtagcagcatgctccagctttgtgaactatggcacaccagatccgactccaggtgcgccatgctgcgttgctatgacgaccctaagcaacatagctagctccaccgaaatgcgccaggccgtctgtagatgtgttatggaccttattactacttacaacccaaatgctactgccattgccactttgcctggtttctgtggtgtttctcttggtttcaccattgaccctaacactgactgtgaatag

SEQ ID NO: 56 - DNA, 462 bp, Nicotiana tabacum
  source 1..462: mol_type = genomic DNA
atggcaagagagaaatctgtaatgtgtatggtgatggttttgggggttgccataataattcagggcactggagctgctgaagaatgtagcacagtgacagcactggtagcagcatgctccagctttgtgaactatggcacaccagatccaactccaggtgcaccatgctgcgtcgctatgacgaccctaagcaacatagctagctccaccgagatgcgccaggccgtctgtagatgcgtgatggaccttattactacttacaacccaaatgctactgccattgccactctgcctggtttctgtggtgtttctcttggtttcaccattgaccctaacactgactccatccgatattcaaggtccactggtcctattaatcgtgatttgctctggttaaagaagttctctactcccgggtgtctcataacaatatggtctctggaaagtgatgctgaagaatga

SEQ ID NO: 57 - DNA, 480 bp, Nicotiana tabacum
  source 1..480: mol_type = genomic DNA
atgaagatgttagctagtgcaagttcaatttttgtagctattgtccttctttcattacatgttagtgctcagtcggattgccaacaagtgatcgttggtttagccccgtgtctgcaatacattcaaggaaatgcgacaaccccatcatcaggatgttgcactcaacttgctactatagtgaaaaatcagccacaatgcttatgtcaagttgttaatggtggtggttcaaatttaggaattaatgttaatcaaacacagactatggctcttcccaaagcttgtaatgtccaaacaccctctattagcctttgcaaaggtactactccttcaggttcaccggggtctccatccactccttcaggtggatcgaagggtgagccaagtggaaattcatcaggaaactcagtcaagatgccatactctctcttatttacccttgtagctatcgcgtttttcgccacagtcttcaccaccatctga

SEQ ID NO: 58 - DNA, 495 bp, Nicotiana tabacum
  source 1..495: mol_type = genomic DNA
atgcagatatcagcgagcacaataagtgcaataatagttgtggcagtggctcttctttcattacatgtcatcagcgctcaatcggattgcgaacaggtggttgttgggttagctccgtgcctgcaatacatagaagggaatgccacgagcccgtcatcaggatgctgcactcagctcgccactatagtgaagacgcagcctcagtgcttgtgtcacgttttgaatggcgagggctcatcctttggagttaatatcaatcagacacttgctttggctcttcctacagcttgtaatgcccacactccctttcttactctatgtaaagcaacttccccaactggttctccggaaatctctccgtccattccttcaggagacagatattcaagattttcgccaaatggagattcattgaagctgcagccatactctctgttatttacccttaatgtagccactttgtcatatgtcacaatattcagctccatcttataa

SEQ ID NO: 59 - DNA, 336 bp, Nicotiana tabacum
  source 1..336: mol_type = genomic DNA
atggcaaaaatccttatagctttgtttgctttgtctctaattttaggccaaacaaatgcagatattcagtgtagtgatgttatatcaaaagtgagctcttgtgaagggtatttgctaggtaaagttgcagcacctagcccaaattgttgctttggattgcaagatttggctaaagtggctgatgactcacaaccagatcgtcaaactatttgtcaatgctttaaagctgccatgcaaactttccctgtggacttccaaaaagctaaacaacttcctcagatttgccatttcaagagtactataccaattgaacccaatgttgattgctcaaagtaa

SEQ ID NO: 60 - DNA, 354 bp, Nicotiana tabacum
  source 1..354: mol_type = genomic DNA
atggctaaagtagcattgttggtggtggtgtgcatggcagcagtagctgtgatgctaacgccccatgcagacgctgacatctcttgtgggcaggttgttgcgagcttgtcaccatgcattagctatgtgaggcaaggtggtgctattccagcaccatgctgcagtggaattaattccctcaacaaccaagctaccagcactcctgatcgacagacggcttgtaactgtattaaatctgctgctgcaggcatcagtggcatcaacttcagtcttgctggtagtcttcctagcaaatgtggtgtcaatcttccctataagattagcccttccattgactgctccacggtgcagtaa

SEQ ID NO: 61 - DNA, 345 bp, Nicotiana tabacum
  source 1..345: mol_type = genomic DNA
atggaaatggtaggtaagattgcatgctttgtggttttgtgcatggtggtggttgcaccccatgcagaggcactgagctgcggccaagttcagtccggcttggctccttgtctcccttatttgcagggccgcggccctttggggagctgttgtggtggtgttaaaggtctgttgggtgcagccaagtccccatctgaccggaagactgcatgcacttgcctgaaatcggctgctaatgctattaagggtattgatatgggcaaagccgctggtctccctggtgcttgtggtgtcaacattccttacaagatcagcccctccacagactgctctaaggtccagtaa

SEQ ID NO: 62 - DNA, 294 bp, Nicotiana tabacum
  source 1..294: mol_type = genomic DNA
atgaagaagggtggtaattcttttgcggcaataatcttggttgtgacactagtcctttttcttggcgaatttctagtgacagaggcagtaacgtgcagtgtcgtggagctgagtccgtgtgccggggcgatctcgtcgccacagccaccctcttcggcatgttgcgctaagttgaaagagcagaagccttgtctttgtgggtacctcaaaaatccaaacctaaggccttatgtcaattctcctaatgccaagagagttgctaaatcctgtggagtacccactcccagctgttag

SEQ ID NO: 63 - DNA, 354 bp, Nicotiana tabacum; identical to SEQ ID NO: 54
  source 1..354: mol_type = genomic DNA

SEQ ID NO: 64 - DNA, 351 bp, Nicotiana tabacum; identical to SEQ ID NO: 55
  source 1..351: mol_type = genomic DNA

SEQ ID NO: 65 - DNA, 655 bp, Nicotiana tabacum
  source 1..655: mol_type = genomic DNA
atggcaagagagaaatctgtaatgtgtatggtgatggttttgggggttgccataataattcagggcactggagctgctgaagaatgtagcacagtgacagcactggtagcagcatgctccagctttgtgaactatggcacaccagatccaactccaggtgcaccatgctgcgtcgctatgacgaccctaagcaacatagctagctccaccgagatgcgccaggccgtctgtagatgcgtgatggaccttattactacttacaacccaaatgctactgccattgccactctgcctggtttctgtggtgtttctcttggtttcaccattgaccctaacactgactgtgaatagtaagtaagacaaccataagtttcttatcacctaatatttcatactttgtttgttggtttagccatccgatattcaaggtccactggtcctattaatcgtgatttgctctggttaaagaagttctctactcccggggtgaaaacccgaaaactttgtcttaagaggtggaggtattctaaccatccaccacacaccttggtggttaatatttcatagttttatcacaatgtctgatcaatcttttcttttctctttgcagtgtctcataacaatatggtctctggaaagtgatgctgaagaatga

SEQ ID NO: 66 - DNA, 1174 bp, Nicotiana tabacum
  source 1..1174: mol_type = genomic DNA
atgaagatgttagctagtgcaagttcaatttttgtagctattgtccttctttcattacatgttagtgctcagtcggattgccaacaagtgatcgttggtttagccccgtgtctgcaatacattcaaggaaatgcgacaaccccatcatcaggatgttgcactcaacttgctactatagtgaaaaatcagccacaatgcttatgtcaagttgttaatggtggtggttcaaatttaggaattaatgttaatcaaacacagactatggctcttcccaaagcttgtaatgtccaaacaccctctattagcctttgcaaaggtatgttatgtttatcaataatattctgctttctttttctcttttcttttcactttttcacttttgttcaaacttgatgtatattttgtataatgacattttctagataaatattttttttaaagaagtcattttttaagaaatacatttccaatcaaaagtgaagaatgacttcccttggacgaaatttgttttccttacaacaatattaagttgtaactttatattaattattacttgttttatttagagattattattaatatttaatgcttaaatttttatcagatttaatattattattattattcattttctactggatattttttgtccaaaaaatatcacttttcaaaaaaatatatttcatggaaaatgacttctgatatgttaaacacactcgagtgaagattttcaagtttataacaagttttaatttgtcttatattatcaatataaacatttcaggtactactccttcaggttcaccggggtctccatccactccttcaggtacggataatacgagcaaaacttctttttctagtaatatatacgaacaaaactcatctttgtctaaaccatacttagcccaccttattacaccgcgtggcttcaagtagttcccctactagtaacacgcgtatcaaacccaccttatctcactgcatgtgtatcaaccctttggatgttattttttcttcctattaacaaaattaatcttgtatacttttggcaggtggatcgaagggtgagccaagtggaaattcatcaggaaactcagtcaagatgccatactctctcttatttacccttgtagctatcgcgtttttcgccacagtcttcaccaccatctga

SEQ ID NO: 67 - DNA, 1817 bp, Nicotiana tabacum
  source 1..1817: mol_type = genomic DNA
atgcagatatcagcgagcacaataagtgcaataatagttgtggcagtggctcttctttcattacatgtcatcagcgctcaatcggattgcgaacaggtggttgttgggttagctccgtgcctgcaatacatagaagggaatgccacgagcccgtcatcaggatgctgcactcagctcgccactatagtgaagacgcagcctcagtgcttgtgtcacgttttgaatggcgagggctcatcctttggagttaatatcaatcagacacttgctttggctcttcctacagcttgtaatgcccacactccctttcttactctatgtaaaggtacgctaaagttatcactacttctttaattcccaaacactagtcgtttgactaactaaattttcttaatatatttgtctttaaaaaaaacttgaagtatttattactttctctgtctcaatttatgtaaagttgattatacggagggtaagaaagaaatataaacctttgaaatttgtattctaaaacacgctttagatatttatatgtctataaattattttattaaggataaaatggatattttgaagtcaattaaattacttccaaaactagaaaggtaatattctttttaggacaaactaaaaagtttttccaaacacatcttaactacaatatccgttaaggacgtttacgggtcgtttggtacatgagataaaaataataattttgggataaagtttgggattaaatttatctcatgtttcatttggaatattagctaatcttgggattaacttttacactaaaattgtgggattagttatccaatatagaaggcgggataactaatcgcatgagatattccaccccataggatattctcggatatcccaccattataccaaacgatccataagagaaaaataaataacaacttataattacttttataattaaagtcattcaatatctttaagggtctgtttggaaagccttgtgtaattacttggttgtgtaattactaggatagtaattacacagcctggtaattacgtagtattgtaattacaatggcttgtttgtttgtcatggtgtaattacagtgtaattacatgtgtcatgtttggttgtacaagtgtaattatataaataaataaatattttttactgtatgataaatatttatatgtataatagatattaaatactattatatattgaaataaaaaaaaggaattggaaaggttgcctcaccaaattctttaccgccttttgagaattggagagtgtaattaccctctcccaattacactcaattcatcaccaatcaaataattacttggttaaataaatatgccaaagtgtataattacacccaattacacttaatttcaatttccatgtggctttccaaacaggctctaaggggtagcaacgtctttgcccttctttctttttattttttcccgaagcttttcatttccctctaattatgacgaaagatcattgttttaatttgtactgtgaatataaacatttcagcaacttccccaactggttctccggaaatctctccgtccattccttcaggtacgaatacgaacaaatcgtgcactgtaacaactgcactttttccatgttttttttcctgcaccaatttcaacatttatataatgggtgtgagaattatatacttgtgagcgcaggagacagatattcaagattttcgccaaatggagattcattgaagctgcagccatactctctgttatttacccttaatgtagccactttgtcatatgtcacaatattcagctccatcttataa

SEQ ID NO: 68 - DNA, 336 bp, Nicotiana tabacum; identical to SEQ ID NO: 59
  source 1..336: mol_type = genomic DNA

SEQ ID NO: 69 - DNA, 354 bp, Nicotiana tabacum; identical to SEQ ID NO: 60
  source 1..354: mol_type = genomic DNA

SEQ ID NO: 70 - DNA, 345 bp, Nicotiana tabacum; identical to SEQ ID NO: 61
  source 1..345: mol_type = genomic DNA

SEQ ID NO: 71 - DNA, 294 bp, Nicotiana tabacum; identical to SEQ ID NO: 62
  source 1..294: mol_type = genomic DNA

SEQ ID NO: 72 - protein, 117 aa, Nicotiana tabacum
MARLFYTMIILLLALATSAIAEPSCKTVAKGIVPCVSYIRGKHHKSDKPSNMCCKGLNDIANVIKNGKDRVAVCKCIKMALSRIHYDPTRITLASQQCHTPSSLPSVGQNTNCARAI

SEQ ID NO: 73 - protein, 116 aa, Nicotiana tabacum
MAREKSVMCMVMVLGVALIIQGTGAAEECSTVTALVAACSSFVNYGTPDPTPGAPCCVAMTTLSNIASSTEMRQAVCRCVMDLITTYNPNATAIATLPGFCGVSLGFTIDPNTDCE

SEQ ID NO: 74 - protein, 153 aa, Nicotiana tabacum
MAREKSVMCMVMVLGVAIIIQGTGAAEECSTVTALVAACSSFVNYGTPDPTPGAPCCVAMTTLSNIASSTEMRQAVCRCVMDLITTYNPNATAIATLPGFCGVSLGFTIDPNTDSIRYSRSTGPINRDLLWLKKFSTPGCLITIWSLESDAEE

SEQ ID NO: 75 - protein, 159 aa, Nicotiana tabacum
MKMLASASSIFVAIVLLSLHVSAQSDCQQVIVGLAPCLQYIQGNATTPSSGCCTQLATIVKNQPQCLCQVVNGGGSNLGINVNQTQTMALPKACNVQTPSISLCKGTTPSGSPGSPSTPSGGSKGEPSGNSSGNSVKMPYSLLFTLVAIAFFATVFTTI

SEQ ID NO: 76 - protein, 164 aa, Nicotiana tabacum
MQISASTISAIIVVAVALLSLHVISAQSDCEQVVVGLAPCLQYIEGNATSPSSGCCTQLATIVKTQPQCLCHVLNGEGSSFGVNINQTLALALPTACNAHTPFLTLCKATSPTGSPEISPSIPSGDRYSRFSPNGDSLKLQPYSLLFTLNVATLSYVTIFSSIL

SEQ ID NO: 77 - protein, 111 aa, Nicotiana tabacum
MAKILIALFALSLILGQTNADIQCSDVISKVSSCEGYLLGKVAAPSPNCCFGLQDLAKVADDSQPDRQTICQCFKAAMQTFPVDFQKAKQLPQICHFKSTIPIEPNVDCSK

SEQ ID NO: 78 - protein, 117 aa, Nicotiana tabacum
MAKVALLVVVCMAAVAVMLTPHADADISCGQVVASLSPCISYVRQGGAIPAPCCSGINSLNNQATSTPDRQTACNCIKSAAAGISGINFSLAGSLPSKCGVNLPYKISPSIDCSTVQ

SEQ ID NO: 79 - protein, 114 aa, Nicotiana tabacum
MEMVGKIACFVVLCMVVVAPHAEALSCGQVQSGLAPCLPYLQGRGPLGSCCGGVKGLLGAAKSPSDRKTACTCLKSAANAIKGIDMGKAAGLPGACGVNIPYKISPSTDCSKVQ

SEQ ID NO: 80 - protein, 97 aa, Nicotiana tabacum
MKKGGNSFAAIILVVTLVLFLGEFLVTEAVTCSVVELSPCAGAISSPQPPSSACCAKLKEQKPCLCGYLKNPNLRPYVNSPNAKRVAKSCGVPTPSC

SEQ ID NO: 81 - protein, 229 aa, synthetic construct
  REGION 1..229: note = Description of Sequence: Synthetic polypeptide
MSNNSLSIKNPKVSLAFVMLPKANMWKETKLKISIMLNKQLFMAKNIMKEMVGSIACLLVLCMVVAAPHAAAPIQRHAAITCGQVDASLAPCLPYLTGGGAPTRGPSAACCSGVKSLLGAAKTTAPDLDRQAACNCLKSAANRIFTGINDDAAAALPSKCGVNYLPFKISPSTEPVDCSKIQMSTPSGDRDGLFSYKFSGDSCKIQPYSLLFTLEVAHAFFATIFPSIL

SEQ ID NO: 82 - protein, 88 aa, Nicotiana tabacum
SAIAEPSCKTVAKGIVPCVSYIRGKHHKSDKPSNMCCKGLNDIANVIKNGKDRVAVCKCIKMALSRIHYDPTRITLASQQCHTPSSLP

SEQ ID NO: 83 - protein, 91 aa, Nicotiana tabacum
ALIIQGTGAAEECSTVTALVAACSSFVNYGTPDPTPGAPCCVAMTTLSNIASSTEMRQAVCRCVMDLITTYNPNATAIATLPGFCGVSLGF

SEQ ID NO: 84 - protein, 91 aa, Nicotiana tabacum
AIIIQGTGAAEECSTVTALVAACSSFVNYGTPDPTPGAPCCVAMTTLSNIASSTEMRQAVCRCVMDLITTYNPNATAIATLPGFCGVSLGF

SEQ ID NO: 85 - protein, 86 aa, Nicotiana tabacum
LLSLHVSAQSDCQQVIVGLAPCLQYIQGNATTPSSGCCTQLATIVKNQPQCLCQVVNGGGSNLGINVNQTQTMALPKACNVQTPSI

SEQ ID NO: 86 - protein, 87 aa, Nicotiana tabacum
LLSLHVISAQSDCEQVVVGLAPCLQYIEGNATSPSSGCCTQLATIVKTQPQCLCHVLNGEGSSFGVNINQTLALALPTACNAHTPFL

SEQ ID NO: 87 - protein, 87 aa, Nicotiana tabacum
LGQTNADIQCSDVISKVSSCEGYLLGKVAAPSPNCCFGLQDLAKVADDSQPDRQTICQCFKAAMQTFPVDFQKAKQLPQICHFKSTI

SEQ ID NO: 88 - protein, 88 aa, Nicotiana tabacum
MLTPHADADISCGQVVASLSPCISYVRQGGAIPAPCCSGINSLNNQATSTPDRQTACNCIKSAAAGISGINFSLAGSLPSKCGVNLPY

SEQ ID NO: 89 - protein, 86 aa, Nicotiana tabacum
VVAPHAEALSCGQVQSGLAPCLPYLQGRGPLGSCCGGVKGLLGAAKSPSDRKTACTCLKSAANAIKGIDMGKAAGLPGACGVNIPY

SEQ ID NO: 90 - protein, 78 aa, Nicotiana tabacum
VLFLGEFLVTEAVTCSVVELSPCAGAISSPQPPSSACCAKLKEQKPCLCGYLKNPNLRPYVNSPNAKRVAKSCGVPTP

SEQ ID NO: 91 - protein, 86 aa, Nicotiana tabacum
VVAPHAEALTCGQVQSSLAPCVPYLLGRGPLGGCCGGVKRLLGAARTPADRKTACNCLKSAANTFKGIDMGNAARLPGTCGVNIPY

SEQ ID NO: 92 - protein, 103 aa, synthetic construct
  REGION 1..103: note = Description of Sequence: Synthetic polypeptide
VAAPHAAAPIQRHAAITCGQVDASLAPCLPYLTGGGAPTRGPSAACCSGVKSLLGAAKTTAPDLDRQAACNCLKSAANRIFTGINDDAAAALPSKCGVNYLPF

SEQ ID NO: 93 - protein, 162 aa, synthetic construct; identical to SEQ ID NO: 22
  REGION 1..162: note = Description of Sequence: Synthetic polypeptide
  VARIANT 1..48: note = This region may be absent

(19) United States
(12) Patent Application Publication
(10) Pub. No.: US 2025/0263735 A1
(43) Pub. Date: Aug. 21, 2025

(54) INCREASING TRICHOME DENSITY AND IMPROVING TRANSPORT OF METABOLITES IN PLANT TRICHOMES

(71) Applicants: Altria Client Services LLC, Richmond, VA (US); University Of Virginia Patent Foundation, Charlottesville, VA (US)

(72) Inventors: Chengalrayan Kudithipudi, Midlothian, VA (US); Yanxin Shen, Henrico, VA (US); Dongmei Xu, Glen Allen, VA (US); Michael Paul Timko, Charlottesville, VA (US); Roel Rabara, Charlottesville, VA (US)

(73) Assignees: Altria Client Services LLC, Richmond, VA (US); University Of Virginia Patent Foundation, Charlottesville, VA (US)

(21) Appl. No.: 19/199,865

(22) Filed: May 6, 2025

Related U.S. Application Data
(63) Continuation of application No. 17/591,057, filed on Feb. 2, 2022, now Pat. No. 12,319,923.
(60) Provisional application No. 63/145,259, filed on Feb. 3, 2021.
(60) Provisional application No. 63/145,262, filed on Feb. 3, 2021.
(60) Provisional application No. 63/145,263, filed on Feb. 3, 2021.

(51) Int. Cl. C12N 15/82; C07K 14/415; C12P 5/00

(52) U.S. Cl. CPC C12N 15/8262; C12N 15/8223; C12N 15/8243; C07K 14/415; C12N 2800/22; C12P 5/007

(57) ABSTRACT

The present disclosure relates to compositions and methods related to modification of trichome density and transport of metabolite and their uses in plants, including tobacco and cannabis. The provided transcription factors enable the increase in trichome density in plants.

### CROSS-REFERENCE TO RELATED APPLICATIONS

This application is a continuation of U.S. patent application Ser. No. 17/591,057, filed Feb. 2, 2022, which claims the benefit of U.S. Provisional Patent Application No. 63/145,259, filed Feb. 3, 2021; U.S. Provisional Patent Application No. 63/145,262, filed Feb. 3, 2021; and U.S. Provisional Patent Application No. 63/145,263, filed Feb. 3, 2021, all of which are incorporated by reference herein in their entireties.

### INCORPORATION OF SEQUENCE LISTING

A sequence listing contained in the file named “P35029US01_SL.xml” which is 147,545 bytes (measured in operating system MS-Windows®), created on May 5, 2025, containing a total number of 93 sequences, starting from SEQ ID NO: 1 to SEQ ID NO: 93, is filed electronically herewith and incorporated by reference in its entirety.

### FIELD

The present disclosure relates to enhancing trichome initiation and development on the surface of plant tissues as well as improving the transport of specialized metabolites into trichomes, the exudation of specialized metabolites from trichomes, and the application of such to change the chemical composition in plants, including tobacco.

### BACKGROUND

Glandular trichomes are epidermal outgrowths in plants that are the site of metabolic compound synthesis and storage. Their presence on stem, leaf, and floral tissues provides protection for plants against various biotic and abiotic stresses. Glandular trichomes also play a role in the biosynthesis, storage, and secretion of specialized or secondary metabolites.

Metabolites produced and secreted by glandular trichomes are often hydrophobic (e.g., fatty acid derivatives, flavonoids, terpenoids). Terpenoids constitute the largest and most diverse class of plant metabolites. The olefinic backbone of terpenoids is made of multiples of the five-carbon (C) isoprene unit, with the major groups being monoterpenes (10C), sesquiterpenes (15C), and diterpenes (20C). These terpenoids are produced through the condensation of five-carbon isoprene units (dimethylallyl diphosphate [DMAPP] and isopentenyl diphosphate [IPP]) most often by the sequential head-to-tail addition of DMAPP to IPP.

The amount of secondary metabolites produced is often tightly correlated to the glandular trichome density present on the plant epidermis (Chalvin et al., Cell, 25:477-487 (2020)). One way to increase the amount of secondary metabolite production in plants is to increase the density of trichomes present on the plant epidermis. Transcriptional regulation of trichome initiation has been shown to involve members of MYB and C2H2 zinc-finger family of transcription factors. Transgenic overexpression of Artemisia annua MYB1 (AaMYB1) was shown to increase trichome density and subsequently the production of artemisin (Matias-Hernandez et al., Plant Journal, 90:520-534 (2017)).

Lipid transfer proteins (LTPs) are important in the transport of specialized metabolites in glandular trichomes. Studies have shown that overexpression of LTPs leads to an increase of exudates in plants glandular trichomes (Choi et al., Plant Journal, 70:480-491 (2012)).

Due to the important role of glandular trichomes in the biosynthesis and secretion of terpenoids, there is a need for a greater understanding of the genes, regulatory factors, and signaling mechanisms involved in the control of trichome initiation and development in plants. It is also important to understand the mode of secretion of these specialized metabolites into the cuticle of trichomes. In this disclosure, candidate genes are provided that can be used to modify trichome density in plants. Modification of trichrome density will also improve transport of specialized metabolites in glandular trichomes.

### SUMMARY

In one aspect, this disclosure provides a modified plant, seed, or plant part, comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

In one aspect, this disclosure provides cured tobacco material from a modified tobacco plant or tobacco plant part comprising a recombinant nucleic acid molecule comprising a nucleic acid sequence at encoding an amino acid sequence least 80% identical or similar to an amino acid sequence selected from the group consisting of SEQ ID NOs: 18-22 and 31-33, wherein said nucleic acid sequence is operably linked to a heterologous promoter.

In one aspect, this disclosure provides a tobacco product comprising material from a modified tobacco plant, tobacco seed, or tobacco plant part comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

In one aspect, this disclosure provides a cannabis product comprising material from a modified cannabis plant, cannabis seed, or cannabis plant part comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

In one aspect, this disclosure provides a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

In one aspect, this disclosure provides a method for producing a plant, the method comprising: (a) obtaining at least one plant comprising a recombinant nucleic acid molecule comprising a nucleic acid sequence at least 80% identical to a sequence selected from the group consisting of SEQ ID NOs: 13-17, 23-30, and 34-36, wherein said nucleic acid sequence is operably linked to a heterologous promoter; (b) crossing said at least one plant with at least one plant of a second variety to produce at least one progeny seed; and (c) selecting said at least one progeny seed produced in step (b), or a plant germinated therefrom, wherein said at least one progeny seed or plant germinated therefrom comprises said recombinant nucleic acid molecule.

In one aspect, this disclosure provides a method of generating a modified plant, the method comprising: (a) introducing a recombinant nucleic acid molecule comprising a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell; (b) selecting at least one plant cell from step (a), wherein the at least one plant cell comprises the recombinant nucleic acid molecule; and (c) regenerating a modified plant from the at least one plant cell selected in step (b).

In one aspect, this disclosure provides a method comprising preparing a tobacco product using cured tobacco material from a modified tobacco plant or part therefrom, wherein the modified tobacco plant or part therefrom comprises a recombinant nucleic acid molecule comprises a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell/

In one aspect, this disclosure provides a method comprising preparing a cannabis product using material from a modified cannabis plant or part therefrom, wherein the modified cannabis plant or part therefrom comprises a recombinant nucleic acid molecule comprises a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell.

In one aspect, this disclosure provides a method comprising transforming a plant cell with a recombinant nucleic acid molecule, wherein the recombinant nucleic acid molecule comprises a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell.

In one aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 80% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions.

### BRIEF DESCRIPTION OF THE SEQUENCES

SEQ ID NOs: 1 to 12 are primers that can used for the cloning of nucleic acid sequences described herein for expression in transgenic plants.

SEQ ID NOs: 13 to 17 are nucleic acids sequences corresponding to the coding sequences for tobacco genes of interest described herein, specifically NtMYB86, NtGIS, NbGIS, and NtLTP1.

SEQ ID NOs: 18 to 22 are amino acid sequences corresponding to the nucleic acid sequences of SEQ ID NOs: 13 to 17.

SEQ ID NOs: 23 to 27 are nucleic acid sequences corresponding to the genomic sequences for tobacco genes of interest described herein, specifically NtMYB86, NtGIS, NbGIS, and NtLTP1.

SEQ ID NOs: 28 to 30 are nucleic acid sequences corresponding to the coding sequences for cannabis genes of interest described herein, specifically MYB61, GIS3, and non-specific lipid transfer protein 1-like.

SEQ ID NOs: 31 to 33 are amino acid sequences corresponding to the nucleic acid sequences of SEQ ID NOs: 28 to 30.

SEQ ID NOs: 34 to 36 are nucleic acid sequences corresponding to the genomic sequences for cannabis genes of interest described herein, specifically MYB61, GIS3, and non-specific lipid transfer protein 1-like.

SEQ ID NOs: 37 is a nucleic acid sequence corresponding to the coding sequence for sweet wormwood (Artemisia annua) specifically MYB transcription factor.

SEQ ID NOs: 38 is an amino acid sequence corresponding to the nucleic acid sequence of SEQ ID NOs: 37.

SEQ ID NOs: 39 to 43 are nucleic acid sequences corresponding to the coding sequences for tobacco genes of interest described herein, specifically MFS, NMD, CBTS2a(HM), GGPPS2 and cis abienol synthase (ABS).

SEQ ID NOs: 44 to 48 are nucleic acid sequences corresponding to the genomic sequences for tobacco genes of interest described herein, specifically MFS, NMD, CBTS2a(HM), GGPPS2 and cis abienol synthase (ABS).

SEQ ID NOs: 49 to 53 are amino acid sequences corresponding to the nucleic acid sequences of SEQ ID NOs:39 to 43.

SEQ ID NOs: 54 to 62 and 63 to 71 are nucleic acid sequences corresponding to coding sequences and genomic sequences, respectively, for tobacco LTP genes.

SEQ ID NOs: 72 to 80 are amino acid sequences corresponding to the nucleic acid sequences of SEQ ID NOs: 54 to 62. SEQ ID NO: 81 is a consensus LTP amino acid sequence. SEQ ID NOs: 82 to 91 are subsequences of SEQ ID NOs: 21 and 72 to 80 as shown in FIG. 9. SEQ ID NO: 92 is the consensus amino acid sequence shown in FIG. 9.

Table 1 provides nucleic acid sequences and amino acid sequences used in this disclosure.

TABLE 1
Sequences used in this disclosure

| SEQ ID NO | Sequence Description | Sequence Type |
|---|---|---|
| 1 | NtMYB86-Fwd | Nucleic acid |
| 2 | NtMYB86-Rev | Nucleic acid |
| 3 | Nb/NtGIS-Fwd | Nucleic acid |
| 4 | Nb/NtGIS-Rev | Nucleic acid |
| 5 | qNtMYB86-Fwd2 | Nucleic acid |
| 6 | qNtMYB86-Rev2 | Nucleic acid |
| 7 | qNbGIS-Fwd | Nucleic acid |
| 8 | qNbGIS-Rev | Nucleic acid |
| 9 | NtLTP1isoform1-Fwd | Nucleic acid |
| 10 | NtLTP1isoform1-Rev | Nucleic acid |
| 11 | NtLTP1isoform2-Fwd | Nucleic acid |
| 12 | NtLTP1isoform2-Rev | Nucleic acid |
| 13 | NtMYB86 (XM_016595116) | Nucleic acid, CDS |
| 14 | NtGIS (XM_016590730) | Nucleic acid, CDS |
| 15 | NbGIS (Niben101Ctg13716g00003) | Nucleic acid, CDS |
| 16 | NtLTP1isoform1 (AB625593) | Nucleic acid, CDS |
| 17 | NtLTP1isoform2 (XM_016578495) | Nucleic acid, CDS |
| 18 | NtMYB86 (XP 016450602) | Amino acid |
| 19 | NtGIS (XP_016446216) | Amino acid |
| 20 | NbGIS | Amino acid |
| 21 | NtLTP1isoform1 (BAK19150) | Amino acid |
| 22 | NtLTP1isoform2 (XP_016433981) | Amino acid |
| 23 | NtMYB86 (XM_016595116) | Nucleic acid, genomic |
| 24 | NtGIS (XM_016590730) | Nucleic acid, genomic |
| 25 | NbGIS (Niben101Ctg13716g00003) | Nucleic acid, genomic |
| 26 | NtLTP1isoform1 (AB625593) | Nucleic acid, genomic |
| 27 | NtLTP1isoform2 (XM_016578495) | Nucleic acid, genomic |
| 28 | MYB61 (Cannabis sativa) | Nucleic acid, CDS |
| 29 | Zinc finger protein GIS3 (Cannabis sativa) | Nucleic acid, CDS |
| 30 | Non-specific lipid-transfer protein 1-like (Cannabis sativa) | Nucleic acid, CDS |
| 31 | MYB61 (Cannabis sativa) | Amino acid |
| 32 | Zinc finger protein GIS3 (Cannabis sativa) | Amino acid |
| 33 | Non-specific lipid-transfer protein 1-like (Cannabis sativa) | Amino acid |
| 34 | MYB61 (Cannabis sativa) | Nucleic acid, genomic |
| 35 | Zinc finger protein GIS3 (Cannabis sativa) | Nucleic acid, genomic |
| 36 | Non-specific lipid-transfer protein 1-like (Cannabis sativa) | Nucleic acid, genomic |
| 37 | MYB transcription factor [Artemisia annua] | Nucleic acid, CDS |
| | KC118530.1 | |
| 38 | MYB transcription factor [Artemisia annua] | Amino acid |
| | AGR40501.1 | |
| 39 | Cytochrome P450_menthofuran synthase (g96188/MFS) | Nucleic acid, CDS |
| 40 | (+)-neomenthol dehydrogenase/ | Nucleic acid, CDS |
| | menthone_reductase_(NMD/g29837) | |
| 41 | Cembratrienol synthase 2a (CBTS-2a/HM/g58533) | Nucleic acid, CDS |
| 42 | Geranylgeranyl diphosphate synthase | Nucleic acid, CDS |
| | (g49326/GGPPS2/GQ911584) | |
| 43 | Cis-abienol synthase_Isoform 1 (g2330/AAB) | Nucleic acid, CDS |
| 44 | Cytochrome P450_menthofuran synthase (g96188/MFS) | Nucleic acid, genomic |
| 45 | (+)-neomenthol dehydrogenase/ | Nucleic acid, genomic |
| | menthone_reductase (NMD/g29837) | |
| 46 | Cembratrienol synthase 2a (CBTS-2a/HM/g58533) | Nucleic acid, genomic |
| 47 | Geranylgeranyl diphosphate synthase | Nucleic acid, genomic |
| | (g49326/GGPPS2/GQ911584) | |
| 48 | Cis-abienol synthase_Isoform 1 (g2330/AAB) | Nucleic acid, genomic |
| 49 | Cytochrome P450_menthofuran synthase (g96188/MFS) | Amino acid |
| 50 | (+)-neomenthol dehydrogenase/ | Amino acid |
| | menthone_reductase (NMD/g29837) | |
| 51 | Cembratrienol synthase 2a (CBTS-2a/HM/g58533) | Amino acid |
| 52 | Geranylgeranyl diphosphate synthase | Amino acid |
| | (g49326/GGPPS2/GQ911584) | |
| 53 | Cis-abienol synthase_Isoform 1 (g2330/AAB) | Amino acid |
| 54 | XM_016601333.1 (XP_016456819.1) | Nucleic acid, CDS |
| 55 | XM_016596613.1 (XP_016452099.1) | Nucleic acid, CDS |
| 56 | XM_016641408.1 (XP_016496894.1) | Nucleic acid, CDS |
| 57 | XM_016607671.1 (XP_016463157.1) | Nucleic acid, CDS |
| 58 | XM_016648588.1 (XP_016504074.1) | Nucleic acid, CDS |
| 59 | XM_016657701.1 (XP_016513187.1) | Nucleic acid, CDS |
| 60 | LTP4 (AB625595.1) | Nucleic acid, CDS |
| 61 | LTP3 (AB625594.1) | Nucleic acid, CDS |
| 62 | LTP2 (AB518680.1) | Nucleic acid, CDS |
| 63 | XM_016601333.1 (XP_016456819.1) | Nucleic acid, genomic |
| 64 | XM_016596613.1 (XP_016452099.1) | Nucleic acid, genomic |
| 65 | XM_016641408.1 (XP_016496894.1) | Nucleic acid, genomic |
| 66 | XM_016607671.1 (XP_016463157.1) | Nucleic acid, genomic |
| 67 | XM_016648588.1 (XP_016504074.1) | Nucleic acid, genomic |
| 68 | XM_016657701.1 (XP_016513187.1) | Nucleic acid, genomic |
| 69 | LTP4 (AB625595.1) | Nucleic acid, genomic |
| 70 | LTP3 (AB625594.1) | Nucleic acid, genomic |
| 71 | LTP2 (AB518680.1) | Nucleic acid, genomic |
| 72 | XP_016456819.1 | Amino acid |
| 73 | XP_016452099.1 | Amino acid |
| 74 | XP_016496894.1 | Amino acid |
| 75 | XP_016463157.1 | Amino acid |
| 76 | XP_016504074.1 | Amino acid |
| 77 | XP_016513187.1 | Amino acid |
| 78 | LTP4 (BAK19152) | Amino acid |
| 79 | LTP3 (BAK19151) | Amino acid |
| 80 | LTP2 (BAJ25798) | Amino acid |
| 81 | LTP Consensus sequence | Amino acid |
| 82 | XP_016456819.1 FIG. 9 sequence | Amino acid |
| 83 | XP_016452099.1 FIG. 9 sequence | Amino acid |
| 84 | XP_016496894.1 FIG. 9 sequence | Amino acid |
| 85 | XP_016463157.1 FIG. 9 sequence | Amino acid |
| 86 | XP_016504074.1 FIG. 9 sequence | Amino acid |
| 87 | XP_016513187.1 FIG. 9 sequence | Amino acid |
| 88 | LPT4_BAK19152 FIG. 9 sequence | Amino acid |
| 89 | LTP3_BAK19151 FIG. 9 sequence | Amino acid |
| 90 | LPT2_BAJ25798 FIG. 9 sequence | Amino acid |
| 91 | LTP1_BAK19150 FIG. 9 sequence | Amino acid |
| 92 | LTP Consensus FIG. 9 sequence | Amino acid |
| 93 | FIG. 8B consensus sequence | Amino acid |

### BRIEF DESCRIPTION OF THE DRAWINGS

FIG. 1 comprises Panels A, B, and C. Panel A depicts glandular trichomes in Nicotiana benthamiana. Panel B depicts glandular trichomes in Nicotiana tabacum variety ‘TN90’, and Panel C depicts glandular trichomes in Nicotiana tabacum variety ‘Izmir Ego’.

FIG. 2 depicts the expression vector used for tobacco transformation.

FIG. 3 comprises panels FIG. 3A and FIG. 3B. FIG. 3A depicts a phylogenetic analysis of the DNA-binding domain of the MYB transcription factor family in tobacco plants (N=319). FIG. 3B depicts protein sequence pairwise alignment of the tobacco MYB86 gene (SEQ ID NO: 18, XP_016450602) and sweet wormwood (Artemisia annua) MYB (SEQ ID NO: 38, AGR40501.1), highlighting the R2 and R3 DNA-binding domains (enclosed in boxes) specifically. The filled circle indicates MYB86 on the cladogram. The scale bar in FIG. 3A represents evolutionary distance used to infer the phylogenetic tree.

FIG. 4 depicts a phylogenetic analysis of the Glabrous Inflorescence Stems (GIS) family of C2H2 transcription factors in tobacco plants (N=247). The filled circle indicates NtGIS gene (SEQ ID NO: 19) on the cladogram. The scale bar represents evolutionary distance used to infer the phylogenetic tree.

FIG. 5 comprises Panels A, B, C, and D. Panel A depicts glandular trichome density in the abaxial leaf midvein of wild-type (WT) TN90. Panel B depicts glandular trichome density in the abaxial midvein of TN90 overexpressing NtMYB86 (SEQ ID NO: 13) under the control of a CaMV 35S promoter, demonstrating an increase in glandular trichrome density compared to wild-type TN90. An abaxial view of the leaf pedicle of wild-type (WT) N. benthamiana is shown in Panel C. Panel D depicts an increase in glandular trichrome density in N. benthamiana overexpressing NtMYB86 under the control of a CaMV 35S promoter as compared to wild-type (Panel C). Images are light micrographs shown at 10× magnification.

FIG. 6 comprises Panel A and Panel B. Panel A depicts the glandular trichome density in the main vegetative stem of wild-type (WT) Nicotiana benthamiana. Panel B depicts the glandular trichome density in the main vegetative stem of N. benthamiana overexpressing NtMYB86 (SEQ ID NO: 18). Images are light micrographs shown at 160× magnification.

FIG. 7 comprises panels FIG. 7A and FIG. 7B. FIG. 7A depicts the light micrographs of glandular trichome density of a tobacco leaf overexpressing NtMYB86 (SEQ ID NO: 18) (b), NtGIS (SEQ ID NO: 19) (c), and NbGIS (SEQ ID NO: 20) (c) in Izmir Ego background compared to a wild-type Nicotiana tabacum variety Izmir Ego (a). Images are shown at 70× magnification with scale bar representing 1 mm. FIG. 7B depicts leaf trichome counts of Izmir Ego WT, Izmir Ego T₀ transgenic lines overexpressing NtMYB86 (SEQ ID NO: 18), NtGIS (SEQ ID NO: 19), and NbGIS (SEQ ID NO: 20).

FIG. 8 comprises panels FIG. 8A and FIG. 8B. FIG. 8A depicts a phylogenetic analysis of the LTP gene family, and the position of the NtLTP1 gene is marked by a filled circle. Publicly available GenBank Accession numbers are provided for each LTP gene family member. FIG. 8B depicts a sequence alignment and gene structure comparison in tobacco plants of two NtLTP1 isoforms (SEQ ID NOs: 21, 22, and 93, respectively, in order of appearance).

FIG. 9 depicts the conserved motifs of a representative number of NtLTP of the 76 that were identified in the Nicotiana tabacum genome following ClustalW multiple sequence alignment algorithm. Six conserved cysteine residues (enclosed in boxes) are notably conserved in >90% of the NtLTPs identified.

FIG. 10 depicts the relative gene expression of NtMYB86 (SEQ ID NO: 14) in nine independent Izmir Ego transgenic lines in the T₀ generation. The relative gene expression was quantified following the 2^{(-ΔΔC(t))} method. See Livak and Schmittgen, Methods, 25:402-408 (2001) for information regarding the 2^{(-ΔΔC(t))} method. Expression of NtMYB86 in a wildtype control Izmir Ego plant is used as a baseline (e.g., wildtype expression is set to 1), and is not shown.

FIG. 11 depicts the relative gene expression of NtGIS (SEQ ID NO: 16) in eight independent Izmir Ego transgenic lines in the T₀ generation. The relative gene expression was quantified following the 2^{-ΔΔCT} method. Expression of NtGIS in a wildtype control Izmir Ego plant is used as a baseline (e.g., wildtype expression is set to 1), and is not shown.

FIG. 12 depicts the relative abundance of duvatrienediol in leaves of T₀ Izmir Ego transgenic lines overexpressing NbGIS (SEQ ID NO: 20), NtGIS (SEQ ID NO: 19), NtMFS (SEQ ID NO: 39), NtNMD (29837; SEQ ID NO: 40), Nicotiana sylvestris cembratrienol synthase 2a (HM; SEQ ID NO: 41), geranylgeranyl diphosphate synthase (GQ; SEQ ID NO: 42), and cis-abienol synthase (AAB; SEQ ID NO: 43).

FIG. 13 depicts the relative abundance of neophytadiene in leaves of T₀ Izmir Ego transgenic lines overexpressing NbGIS (SEQ ID NO: 20), NtGIS (SEQ ID NO: 19), NtMFS (SEQ ID NO: 39), NtNMD (29837; SEQ ID NO: 40), Nicotiana sylvestris cembratrienol synthase 2a (HM; SEQ ID NO: 41), geranylgeranyl diphosphate synthase (GQ; SEQ ID NO: 42), and cis-abienol synthase (AAB; SEQ ID NO: 43).

FIG. 14 depicts the relative abundance of 2,4-Dimethyl-1-heptene in leaves of T₀ Izmir Ego transgenic lines overexpressing NbGIS (SEQ ID NO: 20), NtGIS (SEQ ID NO: 19), NtMFS (SEQ ID NO: 39), NtNMD (29837; SEQ ID NO: 40), Nicotiana sylvestris cembratrienol synthase 2a (HM; SEQ ID NO: 41), geranylgeranyl diphosphate synthase (GQ; SEQ ID NO: 42), and cis-abienol synthase (AAB; SEQ ID NO: 43).

FIG. 15 depicts the relative abundance of cis-abienol in leaves of T₀ Izmir Ego transgenic lines overexpressing NbGIS (SEQ ID NO: 20), NtGIS (SEQ ID NO: 19), NtMFS (SEQ ID NO: 39), NtNMD (29837; SEQ ID NO: 40), Nicotiana sylvestris cembratrienol synthase 2a (HM; SEQ ID NO: 41), geranylgeranyl diphosphate synthase (GQ; SEQ ID NO: 42), and cis-abienol synthase (AAB; SEQ ID NO: 43).

FIG. 16 depicts the relative gene expression levels of NtMYB86 (SEQ ID NO: 13) under control of a CaMV 35S promoter in T₁ transgenic tobacco lines in the Izmir Ego variety.

FIG. 17 depicts the relative gene expression levels of NbGIS (SEQ ID NO: 15) under control of a CaMV 35S promoter in T₁ transgenic tobacco lines in the Izmir Ego variety.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

As used herein, the singular form “a,” “an,” and “the” include plural references unless the context clearly dictates otherwise. For example, the term “a compound” or “at least one compound” may include a plurality of compounds, including mixtures thereof.

Any tobacco plant, or part thereof, provided herein is specifically envisioned for use with any method provided herein. Similarly, any modified tobacco plant, or part thereof, is specifically envisioned for use with any method provided herein. Any nucleic acid sequence, amino acid sequence, or other composition provided herein is specifically envisioned for use with any method provided herein.

Any cannabis plant, or part thereof, provided herein is specifically envisioned for use with any method provided herein. Similarly, any modified cannabis plant, or part thereof, is specifically envisioned for use with any method provided herein. Any nucleic acid sequence, amino acid sequence, or other composition provided herein is specifically envisioned for use with any method provided herein.

Trichomes, in general, are hair-like epidermal outgrowths covering most aerial plant tissues. Trichomes tend to be multicellular, but unicellular trichomes are known as well. Multiple types of trichomes can be found on an individual plant, and trichomes vary in shape, size, and cellular organization. An individual trichome can be classified as a glandular trichome or a non-glandular trichome.

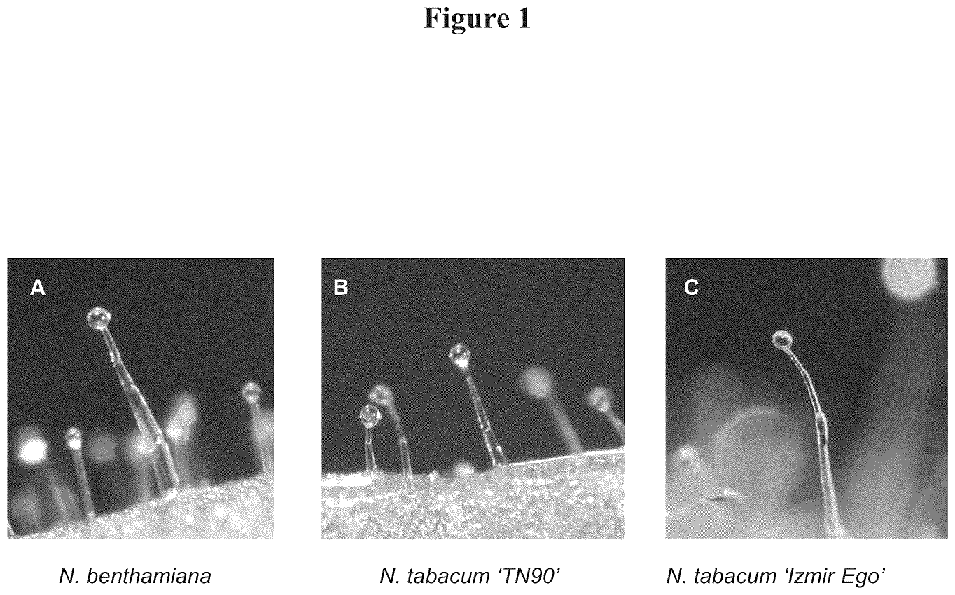

Glandular trichomes (see FIG. 1) are characterized by the presence of a head made of cells that can secrete or store large quantities of specialized metabolites (e.g., terpenes). Within the group of glandular trichomes, a trichome can be further characterized as being peltate or capitate. A capitate glandular trichome typically possesses a stalk with a length that is more than twice the height of the head, and the number of cells in the trichome is highly variable. A peltate trichome is a short-stalked trichome with a large head made of between four and eighteen cells arranged in one or two concentric circles.

In an aspect, a trichome is a glandular trichome. In an aspect, a glandular trichome is a capitate glandular trichome. In an aspect, a glandular trichome is a peltate glandular trichome. In an aspect, a glandular trichome is selected from the group consisting of a capitate glandular trichome and a peltate glandular trichome.

In an aspect, glandular trichome initiation and development is regulated by several transcription factors including, but not limited to, genes belonging to MYB and C2H2 transcription factor gene families.

In an aspect, this disclosure provides a modified plant, seed, or plant part comprising a recombinant nucleic acid molecule comprising a nucleic acid sequence encoding a polypeptide operably linked to a heterologous promoter for the initiation and development of trichome to improve its density. In another aspect, this disclosure provides a modified plant, seed, or plant part comprising a nucleic acid sequence encoding a polypeptide operably linked to a heterologous promoter for improving secretion of metabolites to the trichome cuticles. In a further aspect, a modified plant, seed, or plant part is a plant, seed or plant part of the Nicotiana genus. In a further aspect, a modified plant, seed, or plant part is a plant, seed or plant part of the Cannabis genus.

### Transcription Factors Regulation of Trichome Initiation

Transcription factors are known regulators of various biological processes involved in plants growth and development. Several transcription factors have been identified as regulators of glandular trichome initiation. These include, but are not limited to, the transcription factors gene families R2R3-MYB, HD-ZIP IV, MYC, and C2H2 (Chavlin et al., 2020). Overexpression of MYB1 (AaMYB1) in Artemisia annua significantly increase production of a sesquiterpene lactone, artemisin, as well as increased trichome density (Matias-Hernandez et al., 2017). Another transcription factor that regulates trichome development as well as terpene biosynthesis is MYC1, a basic helix-loop-helix (bHLH). Knockdown of MYC1 (SlMYC1) in tomato led to significant reduction in monoterpenes as well as the ectopic development of smaller Type VI glandular trichomes at low densities (Xu et al., Plant Cell, 30:2988-3005 (2018)). A transcription factor belonging to the C2H2 gene family is also involved in the regulation of trichome development in tobacco. Overexpression of GIS in Nicotiana benthamiana (NbGIS) resulted in the increase of glandular trichome density in transgenic lines (Liu et al., Plant Molecular Biology, 98:153-167 (2018)).

### Storage and Transportation of Metabolites in Glandular Trichome

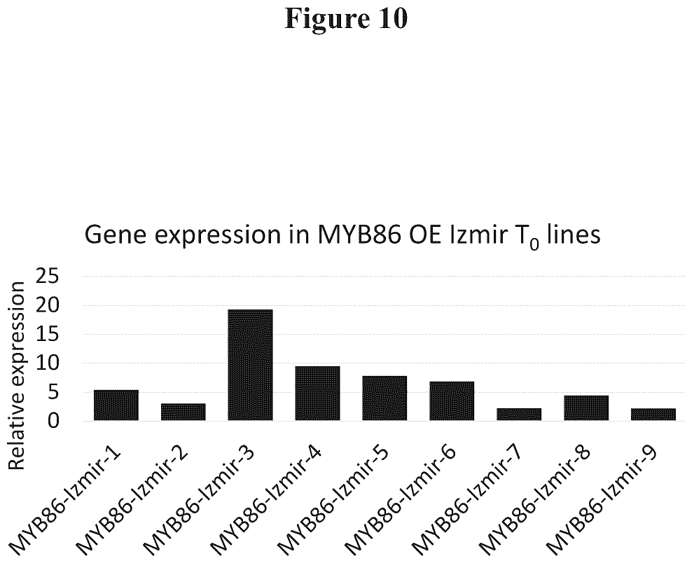

Glandular trichomes produce large amount of metabolites which can account for up to 20% of leaf dry weight (Tissier et al., Trends in Plant Science, 22:930-938 (2017)). Trichomes develop morphological features enabling the storage of secondary metabolites. The type, shape, and size of these morphological features depends on the type of glandular trichomes. Nonvolatile compounds, including diterpenoids, are typically produced by capitate glandular trichomes and directly secreted from the tip of the trichome while glandular trichomes that produce volatile compounds have dedicated structures for secretion and storage (Tissier et al., 2017). Transport of hydrophobic molecules in trichomes requires transporters and lipid transfer proteins (LTPs) that facilitate movement of volatile organic compounds to across hydrophilic cell walls and to prevent VOC repartitioning into the plasma membrane (Tissier et al., 2017). LTPs are small (about 10 kDa), soluble proteins that are characterized by a highly conserved cysteine-rich motif (Salminen et al., Planta, 244:971-997 (2016)). See FIG. 10. LTPs are involved in various functions during plant growth and development including, but not limited to, cuticular wax accumulation, pollen and seed development, and cell expansion (Salminen et al., 2016). The trichome-specific NtLTP1 was reported to increase secretion in trichome exudates in tobacco plants as well increasing protection against insect pests when overexpressed (Choi et al., 2012).

### Plants

In an aspect, a plant provided herein is a modified plant. In an aspect, a seed provided herein is a modified seed. In an aspect, a plant part provided herein is a modified plant part. As used herein, “modified,” in the context of a plant, seed, or plant part, refers to a plant, seed, or plant part, comprising a genetic alteration introduced for certain purposes and beyond natural polymorphisms. Without being limiting, a modified plant, seed, or plant part comprises a recombinant nucleic acid molecule. In another aspect, a modified plant, seed, or plant part comprises a genetic modification. In an aspect, a modified plant, seed, or plant part is a transgenic plant, seed, or plant part.

In an aspect, this disclosure provides a modified plant, seed, or plant part comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 85% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 90% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 95% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 96% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 97% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 98% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 99% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence at least 99.9% identical or similar to an amino acid sequence selected from the group consisting of SEQ ID NOs: 18-22 and 31-33, where the nucleic acid sequence is operably linked to a heterologous promoter. In an aspect, this disclosure provides a modified plant, seed, or plant part comprising a recombinant nucleic acid molecule comprising a nucleic acid sequence encoding an amino acid sequence 100% identical or similar to an amino acid sequence selected from the group consisting of SEQ ID NOs: 18-22 and 31-33, where the nucleic acid sequence is operably linked to a heterologous promoter.

In an aspect, this disclosure provides a modified plant, seed, or plant part comprising a recombinant nucleic acid molecule comprising a nucleic acid sequence encoding an amino acid sequence at least 80%, at least 85%, at least 90%, at least 95%, at least 96%, at least 97%, at least 98%, at least 99%, at least 99.5%, at least 99.9%, or 100% identical or similar to an amino acid sequence selected from the group consisting of SEQ ID NOs: 72-81, where the nucleic acid sequence is operably linked to a heterologous promoter.

In an aspect, this disclosure provides a modified plant, seed, or plant part comprising a recombinant nucleic acid molecule comprising a nucleic acid sequence at least 80% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 85% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 90% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 95% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 96% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 97% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 98% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 99% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence at least 99.9% identical to a nucleic acid sequence selected from the group consisting of SEQ ID NOs: 13-17, 23-30, and 34-36, where the nucleic acid sequence is operably linked to a heterologous promoter. In an aspect, this disclosure provides a modified plant, seed, or plant part comprising a recombinant nucleic acid molecule comprising a nucleic acid sequence 100% identical to a nucleic acid sequence selected from the group consisting of SEQ ID NOs: 13-17, 23-30, and 34-36, where the nucleic acid sequence is operably linked to a heterologous promoter.

In an aspect, this disclosure provides a modified plant, seed, or plant part comprising a recombinant nucleic acid molecule comprising a nucleic acid sequence at least 80%, at least 85%, at least 90%, at least 95%, at least 96%, at least 97%, at least 98%, at least 99%, at least 99.5%, at least 99%, or 100% identical to a nucleic acid sequence selected from the group consisting of SEQ ID NOs: 54-71, where the nucleic acid sequence is operably linked to a heterologous promoter.

In an aspect, at least one leaf of a modified plant comprises a greater average trichome density as compared to a leaf of a control plant grown under comparable conditions. In an aspect, at least one leaf of a modified plant comprises a greater average trichome density on the abaxial side of the leaf as compared to the abaxial side of a leaf of a control plant grown under comparable conditions. In an aspect, at least one leaf of a modified plant comprises a greater average trichome density on the adaxial side of the leaf as compared to the adaxial side of a leaf of a control plant grown under comparable conditions.

In an aspect, at least two leaves of a modified plant comprise a greater average trichome density as compared to two leaves of a control plant grown under comparable conditions. In an aspect, a majority of the leaves of a modified plant comprise a greater average trichome density as compared to the same number of leaves of a control plant grown under comparable conditions. In an aspect, all of the leaves of a modified plant comprise a greater average trichome density as compared to all of the leaves of a control plant grown under comparable conditions.

In an aspect, at least one stem of a modified plant comprises a greater average trichome density as compared to a stem of a control plant grown under comparable conditions. In an aspect, at least one flower of a modified plant comprises a greater average trichome density as compared to a flower of a control plant grown under comparable conditions. In an aspect, at least one root of a modified plant comprises a greater average trichome density as compared to a root of a control plant grown under comparable conditions.

In an aspect, a modified plant comprises a greater average density of glandular trichomes as compared to a control plant. In an aspect, a modified plant comprises a greater average density of non-glandular trichomes as compared to a control plant.

In an aspect, a leaf of a modified tobacco plant comprises at least 70 trichomes per square centimeter. In an aspect, a leaf of a modified tobacco plant comprises at least 75 trichomes per square centimeter. In an aspect, a leaf of a modified tobacco plant comprises at least 80 trichomes per square centimeter. In an aspect, a leaf of a modified tobacco plant comprises at least 85 trichomes per square centimeter. In an aspect, a leaf of a modified tobacco plant comprises at least 90 trichomes per square centimeter. In an aspect, a leaf of a modified tobacco plant comprises at least 95 trichomes per square centimeter. In an aspect, a leaf of a modified tobacco plant comprises at least 100 trichomes per square centimeter. In an aspect, a leaf of a modified tobacco plant comprises at least 105 trichomes per square centimeter. In an aspect, a leaf of a modified tobacco plant comprises at least 110 trichomes per square centimeter. In an aspect, a leaf of a modified tobacco plant comprises at least 115 trichomes per square centimeter. In an aspect, a leaf of a modified tobacco plant comprises at least 120 trichomes per square centimeter. In an aspect, a leaf of a modified tobacco plant comprises at least 125 trichomes per square centimeter. In an aspect, a leaf of a modified tobacco plant comprises at least 130 trichomes per square centimeter. In an aspect, a leaf of a modified tobacco plant comprises at least 140 trichomes per square centimeter. In an aspect, a leaf of a modified tobacco plant comprises at least 150 trichomes per square centimeter.

In an aspect, a leaf of a modified tobacco plant comprises between 60 and 200 trichomes per square centimeter. In an aspect, a leaf of a modified tobacco plant comprises between 60 and 190 trichomes per square centimeter. In an aspect, a leaf of a modified tobacco plant comprises between 60 and 180 trichomes per square centimeter. In an aspect, a leaf of a modified tobacco plant comprises between 60 and 170 trichomes per square centimeter. In an aspect, a leaf of a modified tobacco plant comprises between 60 and 160 trichomes per square centimeter. In an aspect, a leaf of a modified tobacco plant comprises between 60 and 150 trichomes per square centimeter. In an aspect, a leaf of a modified tobacco plant comprises between 60 and 140 trichomes per square centimeter. In an aspect, a leaf of a modified tobacco plant comprises between 60 and 130 trichomes per square centimeter. In an aspect, a leaf of a modified tobacco plant comprises between 60 and 120 trichomes per square centimeter. In an aspect, a leaf of a modified tobacco plant comprises between 60 and 110 trichomes per square centimeter. In an aspect, a leaf of a modified tobacco plant comprises between 60 and 100 trichomes per square centimeter. In an aspect, a leaf of a modified tobacco plant comprises between 60 and 90 trichomes per square centimeter. In an aspect, a leaf of a modified tobacco plant comprises between 60 and 80 trichomes per square centimeter. In an aspect, a leaf of a modified tobacco plant comprises between 60 and 70 trichomes per square centimeter.

In an aspect, a leaf of a modified tobacco plant comprises between 80 and 130 trichomes per square centimeter. In an aspect, a leaf of a modified tobacco plant comprises between 80 and 120 trichomes per square centimeter. In an aspect, a leaf of a modified tobacco plant comprises between 80 and 100 trichomes per square centimeter. In an aspect, a leaf of a modified tobacco plant comprises between 75 and 140 trichomes per square centimeter. In an aspect, a leaf of a modified tobacco plant comprises between 75 and 130 trichomes per square centimeter. In an aspect, a leaf of a modified tobacco plant comprises between 75 and 125 trichomes per square centimeter.

In an aspect, a leaf of a modified tobacco plant comprises at least 1% more trichomes per square centimeter as compared to a control leaf of the same age when grown under comparable conditions. In an aspect, a leaf of a modified tobacco plant comprises at least 5% more trichomes per square centimeter as compared to a control leaf of the same age when grown under comparable conditions. In an aspect, a leaf of a modified tobacco plant comprises at least 10% more trichomes per square centimeter as compared to a control leaf of the same age when grown under comparable conditions. In an aspect, a leaf of a modified tobacco plant comprises at least 15% more trichomes per square centimeter as compared to a control leaf of the same age when grown under comparable conditions. In an aspect, a leaf of a modified tobacco plant comprises at least 20% more trichomes per square centimeter as compared to a control leaf of the same age when grown under comparable conditions. In an aspect, a leaf of a modified tobacco plant comprises at least 25% more trichomes per square centimeter as compared to a control leaf of the same age when grown under comparable conditions. In an aspect, a leaf of a modified tobacco plant comprises at least 30% more trichomes per square centimeter as compared to a control leaf of the same age when grown under comparable conditions. In an aspect, a leaf of a modified tobacco plant comprises at least 40% more trichomes per square centimeter as compared to a control leaf of the same age when grown under comparable conditions. In an aspect, a leaf of a modified tobacco plant comprises at least 50% more trichomes per square centimeter as compared to a control leaf of the same age when grown under comparable conditions. In an aspect, a leaf of a modified tobacco plant comprises at least 60% more trichomes per square centimeter as compared to a control leaf of the same age when grown under comparable conditions. In an aspect, a leaf of a modified tobacco plant comprises at least 70% more trichomes per square centimeter as compared to a control leaf of the same age when grown under comparable conditions. In an aspect, a leaf of a modified tobacco plant comprises at least 80% more trichomes per square centimeter as compared to a control leaf of the same age when grown under comparable conditions. In an aspect, a leaf of a modified tobacco plant comprises at least 90% more trichomes per square centimeter as compared to a control leaf of the same age when grown under comparable conditions. In an aspect, a leaf of a modified tobacco plant comprises at least 100% more trichomes per square centimeter as compared to a control leaf of the same age when grown under comparable conditions. In an aspect, a leaf of a modified tobacco plant comprises at least 110% more trichomes per square centimeter as compared to a control leaf of the same age when grown under comparable conditions. In an aspect, a leaf of a modified tobacco plant comprises at least 125% more trichomes per square centimeter as compared to a control leaf of the same age when grown under comparable conditions. In an aspect, a leaf of a modified tobacco plant comprises at least 150% more trichomes per square centimeter as compared to a control leaf of the same age when grown under comparable conditions. In an aspect, a leaf of a modified tobacco plant comprises at least 200% more trichomes per square centimeter as compared to a control leaf of the same age when grown under comparable conditions.

It will be understood that, unless otherwise specified, a comparison between a modified leaf and a leaf from a control plant grown under comparable conditions must use leaves that are of the same leaf number (e.g., a modified V5 leaf must be compared to a control V5 leaf) on the modified and control plants.

In an aspect, a plant is a tobacco plant. In an aspect, a plant is a Nicotiana plant. In an aspect, a tobacco plant is a Nicotiana tabacum plant.

In an aspect, a Nicotiana plant, seed, or plant part is selected from the group consisting of Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572.

In an aspect, a seed is a tobacco seed. In an aspect, a seed is a Nicotiana seed. In an aspect, a tobacco seed is a Nicotiana tabacum or Nicotiana benthamiana seed.

In an aspect, a plant part is a tobacco plant part. In an aspect, a plant part is a Nicotiana plant part. In an aspect, a tobacco plant part is a Nicotiana tabacum plant part or a Nicotiana benthamiana plant part.

In an aspect, a plant is a cannabis plant. In an aspect, a plant is a Cannabis plant. In an aspect, a cannabis plant is a Cannabis sativa plant. In an aspect, a cannabis plant is a Cannabis indica plant. In an aspect, a cannabis plant is a Cannabis ruderalis plant. In an aspect, a cannabis plant is selected from the group consisting of Cannabis sativa, Cannabis indica, and Cannabis ruderalis.

In an aspect, a seed is a cannabis seed. In an aspect, a seed is a Cannabis seed. In an aspect, a cannabis seed is a Cannabis sativa seed. In an aspect, a cannabis seed is a Cannabis indica seed. In an aspect, a cannabis seed is a Cannabis ruderalis seed. In an aspect, a cannabis seed is selected from the group consisting of Cannabis sativa, Cannabis indica, and Cannabis ruderalis.

In an aspect, a plant part is a cannabis plant part. In an aspect, a plant part is a Cannabis plant part. In an aspect, a cannabis plant part is a Cannabis sativa plant part. In an aspect, a cannabis plant part is a Cannabis indica plant part. In an aspect, a cannabis plant part is a Cannabis ruderalis plant part. In an aspect, a cannabis plant part is selected from the group consisting of Cannabis sativa, Cannabis indica, and Cannabis ruderalis.

In an aspect, a plant part provided includes, but is not limited to, a leaf, a stem, a root, a trichome, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, a plant part does not include a seed. In an aspect, this disclosure provides plant cells, tissues, and organs that are not reproductive material and do not mediate the natural reproduction of the plant. In another aspect, this disclosure also provides plant cells, tissues, and organs that are reproductive material and mediate the natural reproduction of the plant. In another aspect, this disclosure provides plant cells, tissues, and organs that cannot maintain themselves via photosynthesis. In another aspect, this disclosure provides somatic plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, trichome, pod, flower, inflorescence, stalk, pedicel, style, stigma, receptacle, petal, sepal, pollen, anther, filament, ovary, ovule, pericarp, phloem, vascular tissue. In another aspect, this disclosure provides a plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco and cannabis plants naturally reproduce via seeds, not via asexual reproduction or vegetative propagation. In an aspect, this disclosure provides plant endosperm.

This disclosure provides cells from plants provided herein.

As used herein, a “progeny plant” or “progeny seed” can be from any filial generation, e.g., F₁, F₂, F₃, F₄, F₅, F₆, F₇, etc.

In an aspect, a tobacco plant, seed, or plant part, is of a tobacco variety selected from the group consisting of a flue-cured variety, a bright variety, a Burley variety, a Virginia variety, a Maryland variety, a dark variety, a Galpáo variety, an Oriental variety, and a Turkish variety.

In an aspect, a tobacco cell is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a tobacco leaf is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a cured tobacco leaf or plant part is of a tobacco variety selected from the group consisting of a flue cured variety, a bright variety, a Burley variety, a Virginia variety, a Maryland variety, a dark variety, a Galpão variety, an Oriental variety, and a Turkish variety. Skilled artisans further understand that cured tobacco does not constitute a living organism and is not capable of growth or reproduction

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants, seeds, or plant parts provided herein are of a flue-cured tobacco variety selected from the group consisting of the varieties listed in Table 2, and any variety essentially derived from any one of the foregoing varieties. See WO 2004/041006 A1. In a further aspect, tobacco plants, seeds, or plant parts provided herein are in a flue-cured variety selected from the group consisting of K326, K346, and NC196.

TABLE 2
Flue-cured Tobacco Varieties

400 (TC 225)
401 (TC 226)
401 Cherry Red (TC 227)
401 Cherry Red Free (TC 228)
Cash (TC 250)
Cash (TI 278)
CC 101
CC 1063
CC 13
CC 143
CC 200
CC 27
CC 301
CC 33
CC 35
CC 37
CC 400
CC 500
CC 600
CC 65
CC 67
CC 700
CC 800
CC 900
Coker 139 (TC 259)
Coker 139 yb1, yb2
Coker 140 (TC 260)
Coker 176 (TC 262)
Coker 187 (TC 263)
Coker 187-Hicks (TC 265)
Coker 209 (TC 267)
Coker 258 (TC 270)
Coker 298 (TC 272)
Coker 316 (TC 273)
Coker 319 (TC 274)
Coker 347 (TC 275)
Coker 371-Gold (TC 276)
Coker 411 (TC 277)
Coker 48 (TC 253)
Coker 51 (TC 254)
Coker 86 (TC 256)
CU 263 (TC 619)
CU 561
DH95-1562-1
Dixie Bright 101 (TC 290)
Dixie Bright 102 (TC 291)
Dixie Bright 244 (TC 292)
Dixie Bright 27 (TC 288)
Dixie Bright 28 (TC 289)
GF 157
GF 318
GL 26H
GL 338
GL 350
GL 368
GL 395
GL 600
GL 737
GL 939
GL 939 (TC 628)
Hicks (TC 310)
Hicks Broadleaf (TC 311)
K 149 (TC 568)
K 317
K 326
K 326 (TC 319)
K 340 (TC 320)
K 346
K 346 (TC 569)
K 358
K 394 (TC 321)
K 399
K 399 (TC 322)
K 730
Lonibow (TI 1573)
Lonibow (TI 1613)
McNair 10 (TC 330)
McNair 135 (TC 337)
McNair 30 (TC 334)
McNair 373 (TC 338)
McNair 944 (TC 339)
MK94 (TI 1512)
MS K 326
MS NC 71
MS NC 72
NC 100
NC 102
NC 1071 (TC 364)
NC 1125-2
NC 12 (TC 346)
NC 1226
NC 196
NC 2326 (TC 365)
NC 27 NF (TC 349)
NC 291
NC 297
NC 299
NC 37 NF (TC 350)
NC 471
NC 55
NC 567 (TC 362)
NC 60 (TC 352)
NC 606
NC 6140
NC 71
NC 72
NC 729 (TC 557)
NC 810 (TC 659)
NC 82 (TC 356)
NC 8640
NC 89 (TC 359)
NC 92
NC 925
NC 95 (TC 360)
NC 98 (TC 361)
NC EX 24
NC PY 10 (TC 367)
NC TG 61
Oxford 1 (TC 369)
Oxford 1-181 (TC 370)
Oxford 2 (TC 371)
Oxford 207 (TC 632)
Oxford 26 (TC 373)
Oxford 3 (TC 372)
Oxford 414 NF
PD 611 (TC 387)
PVH 03
PVH 09
PVH 1118
PVH 1452
PVH 1600
PVH 2110
PVH 2275
R 83 (Line 256-1) (TI 1400)
Reams 134
Reams 158
Reams 713
Reams 744
Reams M1
RG 11 (TC 600)
RG 13 (TC 601)
RG 17 (TC 627)
RG 22 (TC 584)
RG 8 (TC 585)
RG 81 (TC 618)
RG H51
RG4H 217
RGH 12
RGH 4
RGH 51
RGH 61
SC 58 (TC 400)
SC 72 (TC 403)
Sp. G-168
SPEIGHT 168
Speight 168 (TC 633)
Speight 172 (TC 634)
Speight 178
Speight 179
Speight 190
Speight 196
SPEIGHT 220
SPEIGHT 225
SPEIGHT 227
SPEIGHT 236
Speight G-10 (TC 416)
Speight G-102
Speight G-108
Speight G-111
Speight G-117
Speight G-126
Speight G-15 (TC 418)
Speight G-23
Speight G-28 (TC 420)
Speight G-33
Speight G-41
Speight G-5
Speight G-52
Speight G-58
Speight G-70
Speight G-70 (TC 426)
Speight G-80 (TC 427)
Speight NF3 (TC 629)
STNCB
VA 182
VA 45 (TC 559)
Vesta 30 (TC 439)
Vesta 33 (TC 440)
Vesta 5 (TC 438)
Vesta 62 (TC 441)
Virginia (TI 220)
Virginia (TI 273)
Virginia (TI 877)
Virginia 115 (TC 444)
Virginia 21 (TC 443)
Virginia Bright (TI 964)
Virginia Bright Leaf (TC 446)
Virginia Gold (TC 447)
White Stem Orinoco (TC 451)

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America.

Maryland tobaccos are extremely fluffy, have good burning properties, low nicotine and a neutral aroma. Major Maryland growing countries include the United States of America and Italy.

In one aspect, tobacco plants, seeds, or plant parts provided herein are of a Burley tobacco variety selected from the group consisting of the tobacco varieties listed in Table 3, and any variety essentially derived from any one of the foregoing varieties. In a further aspect, tobacco plants, seeds, or plant parts provided herein are in a Burley variety selected from the group consisting of TN 90, KT 209, KT 206, KT212, and HB 4488.

TABLE 3
Burley Tobacco Varieties

4407 LC
AA-37-1
Burley 21 (TC 7)
Burley 49 (TC 10)
Burley 64 (TC 11)
Burley Mammoth KY 16 (TC 12)
Clay 402
Clay 403
Clay 502
Clays 403
GR 10 (TC 19)
GR 10 (TC 19)
GR 10A (TC 20)
GR 13 (TC 21)
GR 14 (TC 22)
GR 149 LC
GR 153
GR 17 (TC 23)
GR 17B (TC 24)
GR 18 (TC 25)
GR 19 (TC 26)
GR 2 (TC 15)
GR 24 (TC 27)
GR 36 (TC 28)
GR 38 (TC 29)
GR 38A (TC 30)
GR 40 (TC 31)
GR 42 (TC 32)
GR 42C (TC 33)
GR 43 (TC 34)
GR 44 (TC 35)
GR 45 (TC 36)
GR 46 (TC 37)
GR 48 (TC 38)
GR 5 (TC 16)
GR 53 (TC 39)
GR 6 (TC 17)
GR 9 (TC 18)
GR 139 NS
GR 139 S
HB 04P
HB 04P LC
HB 3307P LC
HB 4108P
HB 4151P
HB 4192P
HB 4194P
HB 4196
HB 4488
HB 4488P
HB04P
HB 4488 LC
HIB 21
HPB 21
HY 403
Hybrid 403 LC
Hybrid 404 LC
Hybrid 501 LC
KDH-959 (TC 576)
KDH-960 (TC 577)
KT 200 LC
KT 204 LC
KT 206 LC
KT 209 LC
KT 210 LC
KT 212 LC
KT 215 LC
KY 1 (TC 52)
KY 10 (TC 55)
KY 12 (TC 56)
KY 14 (TC 57)
KY 14 × L8 LC
KY 15 (TC 58)
KY 16 (TC 59)
KY 17 (TC 60)
KY 19 (TC 61)
KY 21 (TC 62)
KY 22 (TC 63)
KY 24 (TC 64)
KY 26 (TC 65)
KY 33 (TC 66)
KY 34 (TC 67)
KY 35 (TC 68)
KY 41A (TC 69)
KY 5 (TC 53)
KY 52 (TC 70)
KY 54 (TC 71)
KY 56 (TC 72)
KY 56 (TC 72)
KY 57 (TC 73)
KY 58 (TC 74)
KY 8654 (TC 77)
KY 8959
KY 9 (TC 54)
KY 907 LC
KY 908 (TC 630)
NBH 98 (Screened)
NC 1206
NC 129
NC 2000 LC
NC 2002 LC
NC 3 LC
NC 5 LC
NC 6 LC
NC 7 LC
NC BH 129 LC
NC03-42-2
Newton 98
R 610 LC
R 630 LC
R 7-11
R 7-12 LC
RG 17
TKF 1801 LC
TKF 2002 LC
TKF 4024 LC
TKF 4028 LC
TKF 6400 LC
TKF 7002 LC
TKS 2002 LC
TN 86 (TC 82)
TN 90 LC
TN 97 Hybrid LC
TN 97 LC
VA 116
VA 119
Virgin A Mutante (TI 1406)
Virginia 509 (TC 84)

In another aspect, tobacco plants, seeds, or plant parts provided herein are of a Maryland tobacco variety selected from the group consisting of the tobacco varieties listed in Table 4, and any variety essentially derived from any one of the foregoing varieties.

TABLE 4
Maryland Tobacco Varieties

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, tobacco plants, seeds, or plant parts provided herein are of a dark air-cured tobacco variety selected from the group consisting of Sumatra, Jatim, Dominican Cubano, Besuki, One sucker, Green River, Virginia sun-cured, and Paraguan Passado, and any variety essentially derived from any one of the foregoing varieties.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants, seeds, or plant parts provided herein are of a dark fire-cured tobacco variety selected from the group consisting of the tobacco varieties listed in Table 5, and any variety essentially derived from any one of the foregoing varieties.

TABLE 5
Dark Fire-Cured Tobacco Varieties

Black Mammoth (TC 461)
Black Mammoth Small Stalk (TC 641)
Certified Madole (TC 463)
D-534-A-1 (TC 464)
DAC ULT 302
DAC ULT 303
DAC ULT 306
DAC ULT 308
DAC ULT 312
DF 300 (TC 465)
DF 485 (TC 466)
DF 516 (TC 467)
DF 911 (TC 468)
DT 508
DT 518 (Screened)
DT 538 LC
DT 592
Improved Madole (TC 471)
Jemigan's Madole (TC 472)
KT 14LC
KT D17LC
KT D4 LC
KT D6 LC
KT D8 LC
KY 153 (TC 216)
KY 157 (TC 217)
KY 160
KY 160 (TC 218)
KY 163 (TC 219)
KY 165 (TC 220)
KY 170 (TC 474)
KY 171 (PhPh)
KY 171 (TC 475)
KY 171 LC
KY 171 NS
KY 180 (TC 573)
KY 190 (TC 574)
Little Crittenden
Little Crittenden (TC 476)
Little Crittenden LC (certified)
Little Crittenden PhPh
Lizard Tail Turtle Foot
Madole (TC 478)
Madole (TC 479)
MS KY 171
MS NL Madole LC
MS TN D950 LC
Nance (TC 616)
Narrow Leaf Madole LC (certified)
Neal Smith Madole (TC 646)
Newtons VH Madole
NL Madole
NL Madole (PhPh)
NL Madole (TC 484)
NL Madole LC
NL Madole LC (PhPh)
NL Madole NS
One Sucker (TC 224)
OS 400
PD 302H
PD 312H
PD 318H
PD 7302 LC
PD 7305
PD 7309 LC
PD 7312 LC
PD 7318 LC
PD 7319 LC
Petico M PG04
PY KY 160 (TC 612)
PY KY 171 (TC 613)
Shirey
TI 1372
TN D94
TN D94 (TC 621)
TN D950
TN D950 (PhPh)
TN D950
TN D950 (TC 622)
TR Madole (TC 486)
VA 309
VA 309 (TC 560)
VA 309 LC (certified)
VA 310 (TC 487)
VA 331 (TC 592)
VA 355 (TC 638)
VA 359
VA 359 (Screened)
VA 359 (TC 639)
VA 359 LC (certified)
VA 403 (TC 580)
VA 405 (TC 581)
VA 409 (TC 562)
VA 510 (TC 572)

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants, seeds, or plant parts provided herein are of an Oriental tobacco variety selected from the group consisting of the tobacco varieties listed in Table 6, and any variety essentially derived from any one of the foregoing varieties.

TABLE 6
Oriental Tobacco Varieties

Bafra (TI 1641)
Bahce (TI 1730)
Bahia (TI 1416)
Bahia (TI 1455)
Baiano (TI 128)
Basma
Basma (TI 1666)
Basma Drama
Basma Hybrid (PhPh)
Basma Zihna I
Bitlis (TI 1667)
Bitlis (TI 1725)
Bubalovac (TI 1282)
Bursa (TI 1650)
Bursa (TI 1668)
Canik (TI 1644)
Djebel 174 (TI 1492)
Djebel 359 (TI 1493)
Djebel 81
Dubec 566 (TI 1409)
Dubec 7 (TI 1410)
Dubek 566 (TI 1567)
Duzce (TI 1670)
Edime (TI 1671)
Ege (TI 1642)
Ege-64 (TI 1672)
Izmir (Akhisar) (TI 1729)
Izmir (Gavurkoy) (TI 1727)
Izmir Ege 64
Izmir-Incekara (TI 1674)
Izmir-Ozbas (TI 1675)
Jaka Dzebel (TI 1326)
Kaba-Kulak
Kagoshima Mamba (TI 158)
Katerini
Katerini S53
Krumovgrad 58
MS Basma
MS Katerini S53
Nevrokop 1146
Ozbas (TI 1645)
Perustitza (TI 980)
Prilep (TI 1291)
Prilep (TI 1325)
Prilep 12-2/1
Prilep 23
Samsun (TC 536)
Samsun 959 (TI 1570)
Samsun Evkaf (TI 1723)
Samsun Holmes NN (TC 540)
Samsun Maden (TI 1647)
Samsun NO 15 (TC 541)
Samsun-BLK SHK Tol (TC 542)
Samsun-Canik (TI 1678)
Samsun-Maden (TI 1679)
Saribaptar 407 - Izmir Region
Smyrna (TC 543)
Smyrna No. 23 (TC 545)
Smyrna No. 9 (TC 544)
Smyma-Blk Shk Tol (TC 546)
Trabzon (TI 1649)
Trabzon (TI 1682)
Trapezund 161 (TI 1407)
Turkish (TC 548)
Turkish Angshit (TI 90)
Turkish Samsum (TI 92)
Turkish Tropizoid (TI 93)
Turkish Varotic (TI 89)
Xanthi (TI 1662)

In an aspect, tobacco plants, seeds, or plant parts provided herein are of a cigar tobacco variety selected from the group consisting of the tobacco varieties listed in Table 7, and any variety essentially derived from any one of the foregoing varieties.

TABLE 7
Cigar Tobacco Varieties

Bahai (TI 62)
Beinhart 1000
Beinhart 1000 (TI 1562)
Beinhart 1000-1 (TI 1561)
Bergerac C
Bergerac C (TI 1529)
Big Cuban (TI 1565)
Castillo Negro, Blanco, Pina (TI 448)
Castillo Negro, Blanco, Pina (TI 448A)
Castillo Negro, Blanco, Pina (TI 449)
Caujaro (TI 893)
Chocoa (TI 289)
Chocoa (TI 313)
Connecticut 15 (TC 183)
Connecticut Broadleaf
Connecticut Broadleaf (TC 186)
Connecticut Shade (TC 188)
Criollo, Colorado (TI 1093)
Enshu (TI 1586)
Florida 301
Florida 301 (TC 195)
PA Broadleaf (TC 119)
Pennsylvania Broadleaf
Pennsylvania Broadleaf (TC 119)
Petite Havana SR1
Petite Havana SR1 (TC 105)

In an aspect, tobacco plants, seeds, or plant parts provided herein are of a tobacco variety selected from the group consisting of the tobacco varieties listed in Table 8, and any variety essentially derived from any one of the foregoing varieties.

TABLE 8
Other Tobacco Varieties

Chocoa (TI 319)
Hoja Parada (TI 1089)
Hoja Parado (Galpoa) (TI 1068)
Perique (St. James Parrish)
Perique (TC 556)
Perique (TI 1374)
Sylvestris (TI 984)
TI 179

In an aspect, a tobacco plant or plant part is from a variety selected from the group consisting of the tobacco varieties listed in Table 2, Table 3, Table 4, Table 5, Table 6, Table 7, and Table 8. In another aspect, a tobacco plant or plant part is from a variety listed in Table 2. In another aspect, a tobacco plant or plant part is from a variety listed in Table 3. In another aspect, a tobacco plant or plant part is from a variety listed in Table 4. In another aspect, a tobacco plant or plant part is from a variety listed in Table 5. In another aspect, a tobacco plant or plant part is from a variety listed in Table 6. In another aspect, a tobacco plant or plant part is from a variety listed in Table 7. In another aspect, a tobacco plant or plant part is from a variety listed in Table 8.

In an aspect, a tobacco seed is from a variety selected from the group consisting of the tobacco varieties listed in Table 2, Table 3, Table 4, Table 5, Table 6, Table 7, and Table 8. In another aspect, a tobacco seed is from a variety listed in Table 2. In another aspect, a tobacco seed is from a variety listed in Table 3. In another aspect, a tobacco seed is from a variety listed in Table 4. In another aspect, a tobacco seed is from a variety listed in Table 5. In another aspect, a tobacco seed is from a variety listed in Table 6. In another aspect, a tobacco seed is from a variety listed in Table 7. In another aspect, a tobacco seed is from a variety listed in Table 8.

In an aspect, a tobacco cell is from a variety selected from the group consisting of the tobacco varieties listed in Table 2, Table 3, Table 4, Table 5, Table 6, Table 7, and Table 8. In another aspect, a tobacco cell is from a variety listed in Table 2. In another aspect, a tobacco cell is from a variety listed in Table 3. In another aspect, a tobacco cell is from a variety listed in Table 4. In another aspect, a tobacco cell is from a variety listed in Table 5. In another aspect, a tobacco cell is from a variety listed in Table 6. In another aspect, a tobacco cell is from a variety listed in Table 7. In another aspect, a tobacco cell is from a variety listed in Table 8.

All foregoing mentioned specific varieties of flue-cured, dark air-cured, Burley, Maryland, dark fire-cured, cigar, or Oriental type are listed only for exemplary purposes. Any additional flue-cured, dark air-cured, Burley, Maryland, dark fire-cured, cigar, or Oriental varieties are also contemplated in the present application.

In an aspect, a plant or variety provided herein is an inbred plant or variety. As used herein, an “inbred” variety is a variety that has been bred for genetic homogeneity.

As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties. In an aspect, a plant or seed provided herein is a hybrid plant or seed.

In an aspect, a tobacco plant provided herein is an inbred tobacco plant. In an aspect, a tobacco seed provided herein is an inbred tobacco seed. In an aspect, a tobacco plant provided herein is a hybrid tobacco plant. In another aspect, a tobacco seed provided herein is a hybrid tobacco seed.

In an aspect, a cannabis plant provided herein is an inbred cannabis plant. In an aspect, a cannabis seed provided herein is an inbred cannabis seed. In an aspect, a cannabis plant provided herein is a hybrid cannabis plant. In an aspect, a cannabis seed provided herein is a hybrid cannabis seed.

Unless specified otherwise, all comparisons to control plants require similar growth conditions or comparable growth conditions for the two plants being compared. As used herein, “grown under comparable conditions,” “similar growth conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103.

As used herein, a “control plant” refers to a plant of identical, or nearly identical, genetic makeup as the modified plant being compared, except for the recombinant nucleic acid molecule provided herein that was introduced to the modified plant.

In an aspect, a plant or variety provided herein is male sterile. In another aspect, a plant or variety provided herein is cytoplasmic male sterile (CMS). Male sterile plants can be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Co., Inc., New York, N.Y. 761 pp.

In another aspect, a plant or variety provided herein is female sterile. As a non-limiting example, female sterile plants can be made by mutating the STIG1 gene. See, for example, Goldman et al. 1994, EMBO Journal 13:2976-2984.

In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 80% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 85% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 90% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 92.5% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 95% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 96% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 97% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 98% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 99% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is at least 99.9% identical to a sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence is sequence selected from the group consisting of SEQ ID NOs: 13-17, 23-30, and 34-36, or a functional fragment thereof; and (b) selecting at least one progeny seed produced in step (a), or a plant germinated therefrom, where the at least one progeny seed or plant germinated therefrom comprises the recombinant nucleic acid molecule.

In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 80% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 85% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 90% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 95% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 96% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 97% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 98% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 99% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence at least 99.9% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule. In an aspect, this disclosure provides a method for producing a plant, the method comprising: (a) crossing at least one plant of a first variety with at least one plant of a second variety to produce at least one progeny seed, where the at least one plant of the first variety comprises a recombinant nucleic acid molecule comprising a promoter operably linked to a heterologous nucleic acid sequence, where the nucleic acid sequence encodes an amino acid sequence 100% identical or similar to an amino acid sequence selected from the group consisting of SEQ ID NOs: 18-22 and 31-33, or a functional fragment thereof; and (b) selecting at least one progeny seed produced in step (a), or a plant germinated therefrom, where the at least one progeny seed or plant germinated therefrom comprises the recombinant nucleic acid molecule.

In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 80% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 85% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 90% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 95% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 96% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 97% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 98% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 99% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence at least 99.9% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide comprises a nucleic acid sequence 100% identical to a nucleic acid sequence selected from the group consisting of SEQ ID NOs: 13-17, 23-30, and 34-36; (b) regenerating a modified plant from the at least one plant cell in step (a), where the modified plant comprises an increased trichome density on at least one leaf as compared to a leaf from a control plant lacking the recombinant nucleic acid when grown under comparable conditions.

In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 80% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 85% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 90% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 92.5% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 95% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 96% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 97% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 98% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 99% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence at least 99.9% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions. In an aspect, this disclosure provides a method for producing a plant with increased trichome density, the method comprising: (a) providing to at least one plant cell a recombinant nucleic acid comprising a heterologous promoter operably linked to a polynucleotide, where the polynucleotide encodes an amino acid sequence 100% identical or similar to an amino acid sequence selected from the group consisting of SEQ ID NOs: 18-22 and 31-33; (b) regenerating a modified plant from the at least one plant cell in step (a), where the modified plant comprises an increased trichome density on at least one leaf as compared to a leaf from a control plant lacking the recombinant nucleic acid when grown under comparable conditions.

In an aspect, a first plant variety and a second plant variety are the same variety. In an aspect, a first plant variety and a second plant variety are two different varieties. In an aspect, a second plant variety comprises a recombinant nucleic acid molecule.

In an aspect, a first plant variety is heterozygous for a recombinant nucleic acid molecule. In an aspect, a first plant variety is hemizygous for a recombinant nucleic acid molecule. In an aspect, a first plant variety is homozygous for a recombinant nucleic acid molecule. In an aspect, a second plant variety is heterozygous for a recombinant nucleic acid molecule. In an aspect, a second plant variety is hemizygous for a recombinant nucleic acid molecule. In an aspect, a second plant variety is homozygous for a recombinant nucleic acid molecule. In an aspect, a progeny seed, or a plant germinated therefrom, is heterozygous for a recombinant nucleic acid molecule. In an aspect, a progeny seed, or a plant germinated therefrom, is hemizygous for a recombinant nucleic acid molecule. In an aspect, a progeny seed, or a plant germinated therefrom, is homozygous for a recombinant nucleic acid molecule.

In an aspect, a first plant variety is a tobacco plant variety. In an aspect, a second plant variety is a tobacco plant variety. In an aspect, a first plant variety is a cannabis plant variety. In an aspect, a second plant variety is a cannabis plant variety.

As used herein, the term “crossing” refers to the deliberate mating of two plants. In an aspect, crossing comprises pollination and/or fertilization of a first plant by a second plant. The two plants being crossed can be distantly related, closely related, or identical. In an aspect, the two plants being crossed are both modified plants. In an aspect, the two plants being crossed are of the same variety. In an aspect, the two plants being crossed are of two different varieties. In an aspect, one of the two plants being crossed is male sterile. In an aspect, one of the two plants being crossed is female sterile. In an aspect, at least one of the two plants being crossed is a hybrid tobacco plant. In an aspect, at least one of the two plants being crossed is a modified plant.

In an aspect, a plant of a first variety is the male parent in a crossing step. In an aspect, a plant of a first variety is the female parent in a crossing step. In an aspect, a plant of a second variety is the male parent in a crossing step. In an aspect, a plant of a second variety is the female parent in a crossing step.

### Nucleic Acids and Amino Acids

As used herein, “heterologous” refers to a sequence (nucleic acid or amino acid) that originates from a foreign species, or, if from the same species, is substantially modified from its native form in composition and/or genomic locus by deliberate human intervention. The term also is applicable to nucleic acid constructs, also referred to herein as “polynucleotide constructs.” In this manner, a “heterologous” nucleic acid construct is intended to mean a construct that originates from a foreign species, or, if from the same species, is substantially modified from its native form in composition and/or genomic locus by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest. It will be appreciated that an endogenous promoter can be considered heterologous to an operably linked endogenous gene if the endogenous promoter and endogenous gene are not naturally operably linked (e.g., human intervention is required to put them in operable linkage). As used herein, an “endogenous” nucleic acid sequence refers to a nucleic acid sequence that occurs naturally in the genome of an organism.

In an aspect, a heterologous polynucleotide comprises a gene. In an aspect, a heterologous polynucleotide encodes a small RNA molecule or a precursor thereof. In an aspect, a heterologous polynucleotide encodes a polypeptide.

As used herein, a “gene” refers to a polynucleotide that can produce a functional unit (e.g., without being limiting, for example, a polypeptide, or a small RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a small RNA molecule or a precursor thereof. In another aspect, a gene encodes a polypeptide.

In an aspect, a gene comprises a nucleic acid sequence at least 80% identical to a nucleic acid sequence selected from the group consisting of SEQ ID NOs: 13-17, 23-30, and 34-36. In an aspect, a gene comprises a nucleic acid sequence at least 85% identical to a nucleic acid sequence selected from the group consisting of SEQ ID NOs: 13-17, 23-30, and 34-36. In an aspect, a gene comprises a nucleic acid sequence at least 90% identical to a nucleic acid sequence selected from the group consisting of SEQ ID NOs: 13-17, 23-30, and 34-36. In an aspect, a gene comprises a nucleic acid sequence at least 92.5% identical to a nucleic acid sequence selected from the group consisting of SEQ ID NOs: 13-17, 23-30, and 34-36. In an aspect, a gene comprises a nucleic acid sequence at least 95% identical to a nucleic acid sequence selected from the group consisting of SEQ ID NOs: 13-17, 23-30, and 34-36. In an aspect, a gene comprises a nucleic acid sequence at least 96% identical to a nucleic acid sequence selected from the group consisting of SEQ ID NOs: 13-17, 23-30, and 34-36. In an aspect, a gene comprises a nucleic acid sequence at least 97% identical to a nucleic acid sequence selected from the group consisting of SEQ ID NOs: 13-17, 23-30, and 34-36. In an aspect, a gene comprises a nucleic acid sequence at least 98% identical to a nucleic acid sequence selected from the group consisting of SEQ ID NOs: 13-17, 23-30, and 34-36. In an aspect, a gene comprises a nucleic acid sequence at least 99% identical to a nucleic acid sequence selected from the group consisting of SEQ ID NOs: 13-17, 23-30, and 34-36. In an aspect, a gene comprises a nucleic acid sequence at least 99.5% identical to a nucleic acid sequence selected from the group consisting of SEQ ID NOs: 13-17, 23-30, and 34-36. In an aspect, a gene comprises a nucleic acid sequence 100% identical to a nucleic acid sequence selected from the group consisting of SEQ ID NOs: 13-17, 23-30, and 34-36.

In an aspect, this disclosure provides a nucleic acid sequence at least 80% identical to a nucleic acid sequence selected from the group consisting of SEQ ID NOs: 13-17, 23-30, and 34-36. In an aspect, this disclosure provides a nucleic acid sequence at least 85% identical to a nucleic acid sequence selected from the group consisting of SEQ ID NOs: 13-17, 23-30, and 34-36. In an aspect, this disclosure provides a nucleic acid sequence at least 90% identical to a nucleic acid sequence selected from the group consisting of SEQ ID NOs: 13-17, 23-30, and 34-36. In an aspect, this disclosure provides a nucleic acid sequence at least 92.5% identical to a nucleic acid sequence selected from the group consisting of SEQ ID NOs: 13-17, 23-30, and 34-36. In an aspect, this disclosure provides a nucleic acid sequence at least 95% identical to a nucleic acid sequence selected from the group consisting of SEQ ID NOs: 13-17, 23-30, and 34-36. In an aspect, this disclosure provides a nucleic acid sequence at least 96% identical to a nucleic acid sequence selected from the group consisting of SEQ ID NOs: 13-17, 23-30, and 34-36. In an aspect, this disclosure provides a nucleic acid sequence at least 97% identical to a nucleic acid sequence selected from the group consisting of SEQ ID NOs: 13-17, 23-30, and 34-36. In an aspect, this disclosure provides a nucleic acid sequence at least 98% identical to a nucleic acid sequence selected from the group consisting of SEQ ID NOs: 13-17, 23-30, and 34-36. In an aspect, this disclosure provides a nucleic acid sequence at least 99% identical to a nucleic acid sequence selected from the group consisting of SEQ ID NOs: 13-17, 23-30, and 34-36. In an aspect, this disclosure provides a nucleic acid sequence at least 99.5% identical to a nucleic acid sequence selected from the group consisting of SEQ ID NOs: 13-17, 23-30, and 34-36. In an aspect, this disclosure provides a nucleic acid sequence 100% identical to a nucleic acid sequence selected from the group consisting of SEQ ID NOs: 13-17, 23-30, and 34-36.

In an aspect, a gene comprises a nucleic acid sequence encoding an amino acid sequence at least 80% identical or similar to an amino acid sequence selected from the group consisting of SEQ ID NOs: 18-22 and 31-33. In an aspect, a gene comprises a nucleic acid sequence encoding an amino acid sequence at least 85% identical or similar to an amino acid sequence selected from the group consisting of SEQ ID NOs: 18-22 and 31-33. In an aspect, a gene comprises a nucleic acid sequence encoding an amino acid sequence at least 90% identical or similar to an amino acid sequence selected from the group consisting of SEQ ID NOs: 18-22 and 31-33. In an aspect, a gene comprises a nucleic acid sequence encoding an amino acid sequence at least 92.5% identical or similar to an amino acid sequence selected from the group consisting of SEQ ID NOs: 18-22 and 31-33. In an aspect, a gene comprises a nucleic acid sequence encoding an amino acid sequence at least 95% identical or similar to an amino acid sequence selected from the group consisting of SEQ ID NOs: 18-22 and 31-33. In an aspect, a gene comprises a nucleic acid sequence encoding an amino acid sequence at least 96% identical or similar to an amino acid sequence selected from the group consisting of SEQ ID NOs: 18-22 and 31-33. In an aspect, a gene comprises a nucleic acid sequence encoding an amino acid sequence at least 97% identical or similar to an amino acid sequence selected from the group consisting of SEQ ID NOs: 18-22 and 31-33. In an aspect, a gene comprises a nucleic acid sequence encoding an amino acid sequence at least 98% identical or similar to an amino acid sequence selected from the group consisting of SEQ ID NOs: 18-22 and 31-33. In an aspect, a gene comprises a nucleic acid sequence encoding an amino acid sequence at least 99% identical or similar to an amino acid sequence selected from the group consisting of SEQ ID NOs: 18-22 and 31-33. In an aspect, a gene comprises a nucleic acid sequence encoding an amino acid sequence at least 99.9% identical or similar to an amino acid sequence selected from the group consisting of SEQ ID NOs: 18-22 and 31-33. In an aspect, a gene comprises a nucleic acid sequence encoding an amino acid sequence 100% identical or similar to an amino acid sequence selected from the group consisting of SEQ ID NOs: 18-22 and 31-33.

In an aspect, this disclosure provides a nucleic acid sequence encoding an amino acid sequence at least 80% identical or similar to an amino acid sequence selected from the group consisting of SEQ ID NOs: 18-22 and 31-33. In an aspect, this disclosure provides a nucleic acid sequence encoding an amino acid sequence at least 85% identical or similar to an amino acid sequence selected from the group consisting of SEQ ID NOs: 18-22 and 31-33. In an aspect, this disclosure provides a nucleic acid sequence encoding an amino acid sequence at least 90% identical or similar to an amino acid sequence selected from the group consisting of SEQ ID NOs: 18-22 and 31-33. In an aspect, this disclosure provides a nucleic acid sequence encoding an amino acid sequence at least 92.5% identical or similar to an amino acid sequence selected from the group consisting of SEQ ID NOs: 18-22 and 31-33. In an aspect, this disclosure provides a nucleic acid sequence encoding an amino acid sequence at least 95% identical or similar to an amino acid sequence selected from the group consisting of SEQ ID NOs: 18-22 and 31-33. In an aspect, this disclosure provides a nucleic acid sequence encoding an amino acid sequence at least 96% identical or similar to an amino acid sequence selected from the group consisting of SEQ ID NOs: 18-22 and 31-33. In an aspect, this disclosure provides a nucleic acid sequence encoding an amino acid sequence at least 97% identical or similar to an amino acid sequence selected from the group consisting of SEQ ID NOs: 18-22 and 31-33. In an aspect, this disclosure provides a nucleic acid sequence encoding an amino acid sequence at least 98% identical or similar to an amino acid sequence selected from the group consisting of SEQ ID NOs: 18-22 and 31-33. In an aspect, this disclosure provides a nucleic acid sequence encoding an amino acid sequence at least 99% identical or similar to an amino acid sequence selected from the group consisting of SEQ ID NOs: 18-22 and 31-33. In an aspect, this disclosure provides a nucleic acid sequence encoding an amino acid sequence at least 99.9% identical or similar to an amino acid sequence selected from the group consisting of SEQ ID NOs: 18-22 and 31-33. In an aspect, this disclosure provides a nucleic acid sequence encoding an amino acid sequence 100% identical or similar to an amino acid sequence selected from the group consisting of SEQ ID NOs: 18-22 and 31-33.

In an aspect, a polypeptide comprises an amino acid sequence at least 80% identical or similar to an amino acid sequence selected from the group consisting of SEQ ID NOs: 18-22 and 31-33. In an aspect, a polypeptide comprises an amino acid sequence at least 85% identical or similar to an amino acid sequence selected from the group consisting of SEQ ID NOs: 18-22 and 31-33. In an aspect, a polypeptide comprises an amino acid sequence at least 90% identical or similar to an amino acid sequence selected from the group consisting of SEQ ID NOs: 18-22 and 31-33. In an aspect, a polypeptide comprises an amino acid sequence at least 92.5% identical or similar to an amino acid sequence selected from the group consisting of SEQ ID NOs: 18-22 and 31-33. In an aspect, a polypeptide comprises an amino acid sequence at least 95% identical or similar to an amino acid sequence selected from the group consisting of SEQ ID NOs: 18-22 and 31-33. In an aspect, a polypeptide comprises an amino acid sequence at least 96% identical or similar to an amino acid sequence selected from the group consisting of SEQ ID NOs: 18-22 and 31-33. In an aspect, a polypeptide comprises an amino acid sequence at least 97% identical or similar to an amino acid sequence selected from the group consisting of SEQ ID NOs: 18-22 and 31-33. In an aspect, a polypeptide comprises an amino acid sequence at least 98% identical or similar to an amino acid sequence selected from the group consisting of SEQ ID NOs: 18-22 and 31-33. In an aspect, a polypeptide comprises an amino acid sequence at least 99% identical or similar to an amino acid sequence selected from the group consisting of SEQ ID NOs: 18-22 and 31-33. In an aspect, a polypeptide comprises an amino acid sequence at least 99.9% identical or similar to an amino acid sequence selected from the group consisting of SEQ ID NOs: 18-22 and 31-33. In an aspect, a polypeptide comprises an amino acid sequence 100% identical or similar to an amino acid sequence selected from the group consisting of SEQ ID NOs: 18-22 and 31-33.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded. A nucleic acid molecule can encode a polypeptide or a small RNA.

As used herein, a “recombinant nucleic acid molecule” refers to a nucleic acid molecule formed by laboratory methods of genetic recombination, such as, without being limiting, molecular cloning. Similarly, a “recombinant DNA construct” refers to a DNA molecule formed by laboratory methods of genetic recombination.

In an aspect, this disclosure provides a recombinant nucleic acid molecule comprising a promoter operably linked to a heterologous nucleic acid sequence, where the nucleic acid sequence is at least 80% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 85% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 90% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 92.5% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 95% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 96% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 97% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 98% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 99% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous nucleic acid sequence, where the nucleic acid sequence is at least 99.9% identical to a sequence selected from the group consisting of SEQ ID NOs: 13-17, 23-30, and 34-36, or a functional fragment thereof. In an aspect, this disclosure provides a recombinant nucleic acid molecule comprising a promoter operably linked to a heterologous polynucleotide, where nucleic acid sequence 100% identical to a sequence selected from the group consisting of SEQ ID NOs: 13-17, 23-30, and 34-36, or a functional fragment thereof.

In an aspect, this disclosure provides a recombinant nucleic acid molecule comprising a promoter operably linked to a heterologous polynucleotide, where the polynucleotide encodes an amino acid sequence at least 80% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 85% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 90% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 95% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 96% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 97% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 98% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 99% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence at least 99.9% identical or similar to an amino acid sequence selected from the group consisting of SEQ ID NOs: 18-22 and 31-33. In an aspect, this disclosure provides a recombinant nucleic acid molecule comprising a promoter operably linked to a heterologous polynucleotide, where the polynucleotide encodes an amino acid sequence 100% identical or similar to an amino acid sequence selected from the group consisting of SEQ ID NOs: 18-22 and 31-33.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

In an aspect, this disclosure provides a promoter. In an aspect, a promoter is heterologous to an operably linked nucleic acid sequence. As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. In an aspect, a promoter is a tissue-preferred promoter. In an aspect, a tissue-preferred promoter is a leaf tissue-preferred promoter. In an aspect, a tissue-preferred promoter is a flower tissue-preferred promoter such as the promoter of gene that encode anthocyanidin synthase (NtANS1) (Lim et al., Plant Cell Tissue Organ Cult 114(3):373-383 (2013)). In an aspect, a tissue-preferred promoter is a root tissue-preferred promoter such as the promoter of extension-like protein (NtREL1) (Zhang et al. Plant Cell Rep 35, 757-769 (2016)). In an aspect, a promoter is an epidermal tissue-preferred promoter such as the promoter of gene that encodes lipid transfer protein (Ntltp1) in root epidermis (Canevascini et al., Plant Physiol 112(2) 513-524 (1996)).

Promoters that drive expression in all or most tissues of the plant are referred to as “constitutive” promoters. In an aspect, a promoter is a constitutive promoter. In an aspect, a constitutive promoter is selected from the group consisting of a Cauliflower Mosaic Virus 35S promoter, a ubiquitin promoter, an actin promoter, an opine promoter, and an alcohol dehydrogenase promoter.

An “inducible” promoter is a promoter that initiates transcription in response to an environmental stimulus such as heat, cold, drought, light, or other stimuli, such as wounding or chemical application. In an aspect, a promoter is an inducible promoter.

In an aspect, a promoter is expressible in a plant cell.

### Terpenes

Terpenes are a class of aromatic organic compounds produced by plants and some insects. Terpenes are hydrocarbon molecules that are often used by plants to either directly deter herbivory or to attract predators or parasites of plant herbivores. Non-limiting examples of terpenes include citral, menthol, camphor, salvinorin A, cannabinoids, and curcuminoids.

In an aspect, a terpene is a terpenoid. Terpenoids (also referred to as isoprenoids) are modified terpenes that contain additional functional groups, which can include oxygen. Terpenoids, which can be cyclic or acyclic, vary in size from five-carbon hemiterpenes to long complex molecules containing thousands of isoprene units. Terpenoids are produced through the condensation of five-carbon isoprene units (e.g., dimethylallyl diphosphate (DMAPP) and isopentenyl diphosphate (IPP)), most often by the sequential head-to-tail addition of DMAPP to IPP. The initial cyclization processes are catalyzed by different terpene synthases and enzyme variation leads to variation in monoterpene structure.

Terpenoids are classified according to the number of isoprene units that comprise the parent terpene. A hemiterpenoid comprises one isoprene unit. A monoterpenoid comprises two isoprene units. A sesquiterpenoid comprises three isoprene units. A diterpenoid comprises four isoprene units. A sesterterpenoid comprises five isoprene units. A triterpenoid comprises six isoprene units. A tetraterpenoid comprises eight isoprene units. A polyterpenoid comprises more than eight isoprene units.

In an aspect, a terpene is menthol. In an aspect, a terpene is menthol or a related compound. In an aspect, a terpene is a labdanoid. In an aspect, a terpene is cembratrienediol. In an aspect, a terpene is levopimaric acid. In an aspect, a terpene is L-leucine. In an aspect, a terpene is neophytadiene. In an aspect, a labdanoid is cis-abienol. In an aspect, a terpene is selected from the group consisting of menthol or a related compound, a labdanoid, cembratrienediol, levopimaric acid, and L-leucine. In an aspect, a terpene is selected from the group consisting of menthol or a related compound, a labdanoid, cembratrienediol, levopimaric acid, L-leucine, and neophytadiene. In an aspect, a terpene is selected from the group consisting of menthol, a labdanoid, cembratrienediol, levopimaric acid, and L-leucine. In an aspect, a terpene is selected from the group consisting of menthol, a labdanoid, cembratrienediol, levopimaric acid, L-leucine, and neophytadiene.

As used herein, “menthol” refers to the organic compound having a chemical formula of C₁₀H₂₀O and the International Union of Pure and Applied Chemistry (IUPAC) name 5-Methyl-2-(propan-2-yl)cyclohexan-1-ol. Menthol is also referred to as “(−)-Menthol.” Related compounds of menthol include, but are not limited to, (+)-Menthol, (+)-Isomenthol, (+)-Neomenthol, (+)-Neoisomenthol, (−)-Isomenthol, (−)-Neomethol, and (−)-Neoisomenthol. In an aspect, a related compound of menthol is selected from the group consisting of (+)-Menthol, (+)-Isomenthol, (+)-Neomenthol, (+)-Neoisomenthol, (−)-Isomenthol, (−)-Neomethol, and (−)-Neoisomenthol.

As used herein, “neophytadiene” refers to the organic compound having a chemical formula of C₂₀H₃₈ and the IUPAC name of 7,11,15-trimethyl-3-methylidenehexadec-1-ene.

As used herein, “cembratrienediol” refers to the organic compound having a chemical formula of C₂₀H₃₄O₂ and the IUPAC name (1R,3R,4Z,8Z,12S,13Z)-1,5,9-trimethyl-12-propan-2-ylcyclotetradeca-4,8,13-triene-1,3-diol. Cembratrienediol is also referred to as “beta-Cembrenediol.”

As used herein, “levopimaric acid” refers to the organic compound having a chemical formula of C₂₀H₃₀O₂ and the IUPAC name (1R,4aR,4bS,10aR)-1,4a-dimethyl-7-propan-2-yl-2,3,4,4b,5,9,10,10a-octahydrophenanthrene-1-carboxylic acid. Levopimaric acid is also referred to as “L-Pimaric acid.”

As used herein, “L-leucine” refers to the amino acid having the chemical formula C₆H₁₂NO₂ and the IUPAC name (2S)-2-amino-4-methylpentanoic acid.

As used herein, a “labdanoid” refers to a terpenoid derivative of the fundamental parent labdane, a diterpene. A labdane has the chemical formula C₂₀H₃₈ and the IUPAC name (1S,2S,4aS,8aR)-2,5,5,8a-tetramethyl-1-[(3R)-3-methylpentyl]-1,2,3,4,4a,6,7,8-octahydronaphthalene.

A non-limiting example of a labdanoid is cis-abienol. As used herein, “cis-abienol” refers to the organic compound having a chemical formula of C₂₀H₃₄O and the IUPAC name (1R,2R,4aS,8aS)-2,5,5,8a-tetramethyl-1-[(2Z)-3-methylpenta-2,4,-dienyl]-3,4,4a,6,7,8-hexahydro-1H-naphthalen-2-ol.

In an aspect, a modified plant, seed, or plant part comprising a recombinant nucleic acid provided herein comprises an increased amount of at least one terpene as compared to a control plant, seed, or plant part lacking the recombinant nucleic acid molecule when grown under comparable conditions. In an aspect, a modified tobacco plant, tobacco seed, or tobacco plant part comprising a recombinant nucleic acid provided herein comprises an increased amount of at least one terpene as compared to a control tobacco plant, tobacco seed, or tobacco plant part lacking the recombinant nucleic acid molecule when grown under comparable conditions. In an aspect, a modified cannabis plant, cannabis seed, or cannabis plant part comprising a recombinant nucleic acid provided herein comprises an increased amount of at least one terpene as compared to a control cannabis plant, cannabis seed, or cannabis plant part lacking the recombinant nucleic acid molecule when grown under comparable conditions.

In an aspect, an increased amount of at least one terpene comprises an increase of at least 0.5%. In an aspect, an increased amount of at least one terpene comprises an increase of at least 1%. In an aspect, an increased amount of at least one terpene comprises an increase of at least 2%. In an aspect, an increased amount of at least one terpene comprises an increase of at least 3%. In an aspect, an increased amount of at least one terpene comprises an increase of at least 4%. In an aspect, an increased amount of at least one terpene comprises an increase of at least 5%. In an aspect, an increased amount of at least one terpene comprises an increase of at least 10%. In an aspect, an increased amount of at least one terpene comprises an increase of at least 12.5%. In an aspect, an increased amount of at least one terpene comprises an increase of at least 15%. In an aspect, an increased amount of at least one terpene comprises an increase of at least 17.5%. In an aspect, an increased amount of at least one terpene comprises an increase of at least 20%. In an aspect, an increased amount of at least one terpene comprises an increase of at least 25%. In an aspect, an increased amount of at least one terpene comprises an increase of at least 30%. In an aspect, an increased amount of at least one terpene comprises an increase of at least 40%. In an aspect, an increased amount of at least one terpene comprises an increase of at least 50%. In an aspect, an increased amount of at least one terpene comprises an increase of at least 60%. In an aspect, an increased amount of at least one terpene comprises an increase of at least 70%. In an aspect, an increased amount of at least one terpene comprises an increase of at least 80%. In an aspect, an increased amount of at least one terpene comprises an increase of at least 90%. In an aspect, an increased amount of at least one terpene comprises an increase of at least 100%. In an aspect, an increased amount of at least one terpene comprises an increase of at least 150%. In an aspect, an increased amount of at least one terpene comprises an increase of at least 200%. In an aspect, an increased amount of at least one terpene comprises an increase of at least 250%. In an aspect, an increased amount of at least one terpene comprises an increase of at least 500%.

In an aspect, an increased amount of at least one terpene comprises an increase of between 0.5% and 500%. In an aspect, an increased amount of at least one terpene comprises an increase of between 0.5% and 250%. In an aspect, an increased amount of at least one terpene comprises an increase of between 0.5% and 100%. In an aspect, an increased amount of at least one terpene comprises an increase of between 0.5% and 75%. In an aspect, an increased amount of at least one terpene comprises an increase of between 0.5% and 50%. In an aspect, an increased amount of at least one terpene comprises an increase of between 0.5% and 25%. In an aspect, an increased amount of at least one terpene comprises an increase of between 0.5% and 10%. In an aspect, an increased amount of at least one terpene comprises an increase of between 0.5% and 5%. In an aspect, an increased amount of at least one terpene comprises an increase of between 0.5% and 500%. In an aspect, an increased amount of at least one terpene comprises an increase of between 5% and 250%. In an aspect, an increased amount of at least one terpene comprises an increase of between 5% and 100%. In an aspect, an increased amount of at least one terpene comprises an increase of between 5% and 50%. In an aspect, an increased amount of at least one terpene comprises an increase of between 25% and 500%. In an aspect, an increased amount of at least one terpene comprises an increase of between 25% and 250%. In an aspect, an increased amount of at least one terpene comprises an increase of between 50% and 100%. In an aspect, an increased amount of at least one terpene comprises an increase of between 100% and 500%.

The amount of terpenes in a plant can be measured using any method known in the art, including, without being limiting, gas chromatography mass spectrometry (GC-MS), Nuclear Magnetic Resonance Spectroscopy, and liquid chromatography-linked mass spectrometry. See The Handbook of Plant Metabolomics, edited by Weckwerth and Kahl, (Wiley-Blackwell) (May 28, 2013). In an aspect, an amount of at least one terpene refers to the concentration of the at least one terpene in the tissue sampled.

### Products

In an aspect, this disclosure provides cured plant material from any plant or plant part provided herein. In an aspect, this disclosure provides cured tobacco material from any tobacco plant or tobacco plant part provided herein.

In an aspect, cured plant material is made by a curing process selected from the group consisting of flue curing, air curing, fire curing, and sun curing. In an aspect, cured tobacco material is made by a curing process selected from the group consisting of flue curing, air curing, fire curing, and sun curing.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In one aspect, tobacco plants or plant components provided herein can be cured using conventional means, (e.g., flue-cured, barn-cured, fire-cured, air-cured or sun-cured). See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation.

Information regarding the harvesting of burley and dark tobacco varieties can be found in the 2019-2020 Burley and Dark Tobacco Production Guide (December 2018) published by the University of Kentucky, The University of Tennessee, Virginia Tech, and North Carolina State University, which is incorporated herein by reference in its entirety.

In an aspect, cured tobacco material comprises tobacco material selected from the group selected from cured leaf material, cured stem material, cured bud material, cured flower material, and cured root material. In an aspect, cured tobacco material comprises cured leaf material, cured stem material, or both. In an aspect, cured tobacco material comprises cured leaf material. In an aspect, cured tobacco material comprises cured stem material.

In an aspect, cured tobacco material comprises flue-cured tobacco material. In an aspect, cured tobacco material comprises air-cured tobacco material. In an aspect, cured tobacco material comprises fire-cured tobacco material. In an aspect, cured tobacco material comprises sun-cured tobacco material. In an aspect, cured tobacco material provided herein is selected from the group consisting of air-cured tobacco material, fire-cured tobacco material, sun-cured tobacco material, and flue-cured tobacco material. In an aspect, cured tobacco material is from a tobacco variety selected from the group consisting of a flue-cured variety, a bright variety, a Burley variety, a Virginia variety, a Maryland variety, a dark variety, an Oriental variety, and a Turkish variety.

In an aspect, cured tobacco leaf provided herein is selected from the group consisting of air-cured tobacco leaf, fire-cured tobacco leaf, sun-cured tobacco leaf, and flue-cured tobacco leaf. In an aspect, cured tobacco leaf is from a tobacco variety selected from the group consisting of a flue-cured variety, a bright variety, a Burley variety, a Virginia variety, a Maryland variety, a dark variety, an Oriental variety, and a Turkish variety.

Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In an aspect, this disclosure provides fermented tobacco material from any tobacco plant, or part thereof, provided herein. In another aspect, this disclosure provides fermented tobacco material from any modified tobacco plant, or part thereof, provided herein.

Tobacco material obtained from the tobacco lines, varieties or hybrids of the present disclosure can be used to make tobacco products. As used herein, “tobacco product” is defined as any product made or derived from tobacco that is intended for human use or consumption. In an aspect, this disclosure provides a tobacco product comprising plant material from a tobacco plant provided herein. In another aspect, this disclosure provides a tobacco product comprising plant material from a modified tobacco plant provided herein. In another aspect, this disclosure provides a tobacco product comprising cured tobacco material. In another aspect, this disclosure provides a tobacco product comprising fermented tobacco material. In another aspect, this disclosure provides a tobacco product comprising a tobacco blend.

Tobacco products include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In an aspect, a tobacco product comprises reconstituted tobacco. In another aspect, this disclosure provides reconstituted tobacco comprising cured tobacco material. As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

In an aspect, a tobacco product comprises expanded tobacco. As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a kretek, a bidi cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco.

In an aspect, a tobacco product of the present disclosure is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.

In an aspect, a tobacco product of the present disclosure is a smokeless tobacco product. In an aspect, a smokeless tobacco product is selected from the group consisting of loose leaf chewing tobacco, plug chewing tobacco, moist snuff, nasal snuff, dry snuff, and snus.

Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat-treated smokeless tobacco. Dry snuff is finely ground tobacco that is placed in the mouth or used nasally.

In yet another aspect, a tobacco product of the present disclosure is selected from the group consisting of an electronically heated cigarette, an e-cigarette, an electronic vaporing device.

In an aspect, a tobacco product of the present disclosure can be a blended tobacco product.

In another aspect, this disclosure provides a tobacco blend comprising cured tobacco material. A tobacco blend can comprise any combination of cured tobacco, uncured tobacco, fermented tobacco, unfermented tobacco, expanded tobacco, and reconstituted tobacco.

In an aspect, a tobacco blend comprises at least 5% cured tobacco by weight. In an aspect, a tobacco blend comprises at least 10% cured tobacco by weight. In an aspect, a tobacco blend comprises at least 15% cured tobacco by weight. In an aspect, a tobacco blend comprises at least 20% cured tobacco by weight. In an aspect, a tobacco blend comprises at least 25% cured tobacco by weight. In an aspect, a tobacco blend comprises at least 30% cured tobacco by weight. In an aspect, a tobacco blend comprises at least 35% cured tobacco by weight. In an aspect, a tobacco blend comprises at least 40% cured tobacco by weight. In an aspect, a tobacco blend comprises at least 45% cured tobacco by weight. In an aspect, a tobacco blend comprises at least 50% cured tobacco by weight. In an aspect, a tobacco blend comprises at least 55% cured tobacco by weight. In an aspect, a tobacco blend comprises at least 60% cured tobacco by weight. In an aspect, a tobacco blend comprises at least 65% cured tobacco by weight. In an aspect, a tobacco blend comprises at least 70% cured tobacco by weight. In an aspect, a tobacco blend comprises at least 75% cured tobacco by weight. In an aspect, a tobacco blend comprises at least 80% cured tobacco by weight. In an aspect, a tobacco blend comprises at least 85% cured tobacco by weight. In an aspect, a tobacco blend comprises at least 90% cured tobacco by weight. In an aspect, a tobacco blend comprises at least 95% cured tobacco by weight.

In an aspect, a tobacco blend comprises at least 5% cured tobacco by volume. In an aspect, a tobacco blend comprises at least 10% cured tobacco by volume. In an aspect, a tobacco blend comprises at least 15% cured tobacco by volume. In an aspect, a tobacco blend comprises at least 20% cured tobacco by volume. In an aspect, a tobacco blend comprises at least 25% cured tobacco by volume. In an aspect, a tobacco blend comprises at least 30% cured tobacco by volume. In an aspect, a tobacco blend comprises at least 35% cured tobacco by volume. In an aspect, a tobacco blend comprises at least 40% cured tobacco by volume. In an aspect, a tobacco blend comprises at least 45% cured tobacco by volume. In an aspect, a tobacco blend comprises at least 50% cured tobacco by volume. In an aspect, a tobacco blend comprises at least 55% cured tobacco by volume. In an aspect, a tobacco blend comprises at least 60% cured tobacco by volume. In an aspect, a tobacco blend comprises at least 65% cured tobacco by volume. In an aspect, a tobacco blend comprises at least 70% cured tobacco by volume. In an aspect, a tobacco blend comprises at least 75% cured tobacco by volume. In an aspect, a tobacco blend comprises at least 80% cured tobacco by volume. In an aspect, a tobacco blend comprises at least 85% cured tobacco by volume. In an aspect, a tobacco blend comprises at least 90% cured tobacco by volume. In an aspect, a tobacco blend comprises at least 95% cured tobacco by volume.

In an aspect, this disclosure provides a cannabis product comprising material from a cannabis plant, cannabis seed, or cannabis plant part provided herein. In an aspect, a cannabis product is a smokeless product. In a further aspect, a smokeless cannabis product is an edible cannabis product. In a further aspect, a smokeless cannabis product is a fiber based product. In an aspect, a cannabis product is a smokable product. In an aspect, a cannabis product is derived from cannabis biomass. In an aspect, a cannabis product is a distillate derived from cannabis biomass.

In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 80% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 85% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 90% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 92.5% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 95% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 96% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 97% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 98% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 99% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence is at least 99.9% identical to a sequence selected from the group consisting of SEQ ID NOs: 13-17, 23-30, and 34-36, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous polynucleotide, where the nucleic acid sequence is selected from the group consisting of SEQ ID NOs: 13-17, 23-30, and 34-36, or a functional fragment thereof.

In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 80% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 85% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 90% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 92.5% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 95% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 96% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 97% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 98% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 99% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence at least 99.9% identical or similar to a sequence selected from the group consisting of SEQ ID NOs: 18-22 and 31-33, or a functional fragment thereof. In an aspect, this disclosure provides a method comprising preparing a tobacco product producing using cured tobacco material from a modified tobacco plant or part therefrom, where the modified tobacco plant or part therefrom comprises a recombinant nucleic acid molecule comprising a promoter operably linked to a heterologous nucleic acid sequence, where the nucleic acid sequence encodes an amino acid sequence selected from the group consisting of SEQ ID NOs: 18-22 and 31-33, or a functional fragment thereof.

In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 80% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 85% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 90% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 92.5% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 95% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 96% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 97% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 98% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 99% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence is at least 99.9% identical to a sequence selected from the group consisting of SEQ ID NOs: 13-17, 23-30, and 34-36, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous polynucleotide, where nucleic acid sequence 100% identical to a sequence selected from the group consisting of SEQ ID NOs: 13-17, 23-30, and 34-36, or a functional fragment thereof.

In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 80% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 85% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 90% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 92.5% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 95% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 96% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 97% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 98% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 99% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence at least 99.9% identical or similar to a sequence selected from the group consisting of SEQ ID NOs: 18-22 and 31-33, or a functional fragment thereof. In an aspect, this disclosure provides a method comprising preparing a cannabis product using material from a modified cannabis plant or part therefrom, where the modified cannabis plant or part therefrom comprises a recombinant nucleic acid molecule comprising a promoter operably linked to a heterologous nucleic acid sequence, where the nucleic acid sequence encodes an amino acid sequence selected from the group consisting of SEQ ID NOs: 18-22 and 31-33, or a functional fragment thereof. Transformation

In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 80% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 85% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 90% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 92.5% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 95% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 96% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 97% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 98% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 99% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is at least 99.9% identical to a sequence selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence is selected from the group consisting of SEQ ID NOs: 13-17, 23-30, and 34-36, or a functional fragment thereof; (b) selecting at least one plant cell from step (a), where the at least one plant cell comprises the recombinant nucleic acid molecule; and (c) regenerating a modified plant from the at least one plant cell selected in step (b).

In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 80% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 85% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 90% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 92.5% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 95% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 96% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 97% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 98% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 99% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence is at least 99.9% identical to a sequence selected from the group consisting of SEQ ID NOs: 13-17, 23-30, and 34-36, or a functional fragment thereof. In an aspect, a method provided herein comprises transforming a plant cell with a recombinant nucleic acid molecule, where the recombinant nucleic acid molecule comprises a promoter operably linked to a heterologous polynucleotide, where nucleic acid sequence 100% identical to a sequence selected from the group consisting of SEQ ID NOs: 13-17, 23-30, and 34-36, or a functional fragment thereof.

In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 80% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 85% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 90% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 92.5% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 95% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 96% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 97% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 98% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 99% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where the nucleic acid sequence encodes an amino acid sequence at least 99.9% identical or similar to a sequence selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b). In an aspect, this disclosure provides a method of generating a modified plant, the method comprising: (a) introducing a recombinant nucleic acid molecule comprising a promoter operably linked to a heterologous nucleic acid sequence to at least one plant cell, where nucleic acid sequence encodes a protein selected from the group consisting of SEQ ID NOs: 18-22 and 31-33, or a functional fragment thereof; (b) selecting at least one plant cell from step (a), where the at least one plant cell comprises the recombinant nucleic acid molecule; and (c) regenerating a modified plant from the at least one plant cell selected in step (b).

In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 80% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 85% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 90% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 92.5% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 95% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 96% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 97% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 98% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 99% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence at least 99.9% identical or similar to a sequence selected from the group consisting of SEQ ID NOs: 18-22 and 31-33, or a functional fragment thereof. In an aspect, a method provided herein comprises transforming a plant cell with a recombinant nucleic acid molecule, where the recombinant nucleic acid molecule comprises a promoter operably linked to a heterologous nucleic acid sequence, where the nucleic acid sequence encodes an amino acid sequence selected from the group consisting of SEQ ID NOs: 18-22 and 31-33, or a functional fragment thereof.

Numerous methods for “introducing” a recombinant nucleic acid molecule to a plant cell are known in the art, which can be used according to methods of the present application to produce a modified plant cell, plant, seed, or plant part. As used herein, the terms “introducing” and “transforming” can be used interchangeably. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, polyethylene glycol (PEG)-mediated transformation, etc., are also known in the art. Modified plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell (e.g., tobacco cell, cannabis cell) with any of the nucleic acid molecules provided herein.

In an aspect, a method of introducing a recombinant nucleic acid molecule to a plant cell comprises Agrobacterium-mediated transformation. In another aspect, a method of introducing a recombinant nucleic acid molecule to a plant cell comprises PEG-mediated transformation. In another aspect, a method of introducing a recombinant nucleic acid molecule to a plant cell comprises biolistic transformation. In another aspect, a method of introducing a recombinant nucleic acid molecule to a plant cell comprises liposome-mediated transfection (lipofection). In another aspect, a method of introducing a recombinant nucleic acid molecule to a plant cell comprises lentiviral transfection.

Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration).

Any plant cell from which a fertile plant can be regenerated is contemplated as a useful recipient cell for practice of this disclosure.

In an aspect, a recombinant nucleic acid molecule is introduced to a tobacco cell. In an aspect, a recombinant nucleic acid molecule is introduced to a tobacco protoplast cell. In another aspect, a recombinant nucleic acid molecule is introduced to a tobacco callus cell. In an aspect, a recombinant nucleic acid molecule is introduced to a tobacco cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

In an aspect, a recombinant nucleic acid molecule is introduced to a cannabis cell. In an aspect, a recombinant nucleic acid molecule is introduced to a cannabis protoplast cell. In another aspect, a recombinant nucleic acid molecule is introduced to a cannabis callus cell. In an aspect, a recombinant nucleic acid molecule is introduced to a cannabis cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference.

### Embodiments

The following examples of non-limiting embodiments are envisioned:

1. A modified plant, seed, or plant part, comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

2. The modified plant, seed, or plant part, of embodiment 1, wherein said nucleic acid sequence comprises a sequence at least 90% identical or similar to a sequence selected from the group consisting of SEQ ID NOs: 13-17, 23-30, and 34-36.

3. The modified plant, seed, or plant part, of embodiment 1 or 2, wherein said nucleic acid sequence is at least 95% identical to a sequence selected from the group consisting of SEQ ID NOs: 13-17, 23-30, and 34-36.

4. The modified plant, seed, or plant part, of any one of embodiments 1-3, wherein said nucleic acid sequence encodes a protein at least 90% identical or similar to a sequence selected from the group consisting of SEQ ID NOs: 18-22 and 31-33.

5. The modified plant, seed, or plant part, of any one of embodiments 1-4, wherein said nucleic acid sequence is selected from the group consisting of SEQ ID NOs: 13-17, 23-30, and 34-36.

6. The modified plant, seed, or plant part, of any one of embodiments 1-5, wherein said amino acid sequence is selected from the group consisting of SEQ ID NOs: 18-22 and 31-33.

7. The modified plant, seed, or part thereof of any one of embodiments 1-6, wherein said modified plant, seed, or plant part is a tobacco plant, tobacco seed, or tobacco plant part.

8. The modified plant, seed, or part thereof of embodiment 7, wherein the tobacco plant, tobacco seed, or plant part is of a tobacco variety selected from the group consisting of a flue-cured variety, a bright variety, a Burley variety, a Virginia variety, a Maryland variety, a dark variety, a Galpão variety, an Oriental variety, and a Turkish variety.

9. The modified plant, seed, or part thereof of any one of embodiments 1-8, wherein the plant is male sterile or cytoplasmically male sterile.

10. The modified plant, seed, or part thereof of any one of embodiments 1-6, wherein said modified plant, seed, or plant part is a cannabis plant, cannabis seed, or cannabis plant part.

11. The modified plant, seed, or plant part, of any one of embodiments 1-10, wherein said plant or plant part comprises an amount of at least one terpene that is increased by at least 5% as compared to a control plant lacking said recombinant nucleic acid molecule.

12. The modified plant, seed, or plant part, of any one of embodiments 1-11, wherein the heterologous promoter is selected from the group consisting of a constitutive promoter, an inducible promoter, and a tissue-preferred or tissue-specific promoter.

13. The modified plant, seed, or plant part of any one of embodiments 1-12, wherein at least one leaf of the modified plant comprises a greater average trichome density as compared to a leaf of a control plant grown under comparable conditions.

14. The modified plant, seed, or plant part of any one of embodiments 1-12, wherein at least one leaf of the modified plant comprises a greater average trichome density on the abaxial side of the leaf as compared to the abaxial side of a leaf of a control plant grown under comparable conditions.

15. The modified plant, seed, or plant part of any one of embodiments 1-12, wherein at least one leaf of the modified plant comprises a greater average trichome density on the adaxial side of the leaf as compared to the adaxial side of a leaf of a control plant grown under comparable conditions.

16. Cured tobacco material from a modified tobacco plant or tobacco plant part comprising a recombinant nucleic acid molecule comprising a nucleic acid sequence at encoding an amino acid sequence least 80% identical or similar to an amino acid sequence selected from the group consisting of SEQ ID NOs: 18-22 and 31-33, wherein said nucleic acid sequence is operably linked to a heterologous promoter.

17. The cured tobacco material of embodiment 16, wherein the cured tobacco material is selected from the group consisting of flue cured tobacco material, air cured tobacco material, fire cured tobacco material, and sun cured tobacco material.

18. A tobacco product comprising the cured tobacco material of embodiment 16.

19. The tobacco product of embodiment 18 or 23, wherein the tobacco product is selected from the group consisting of a kretek, a bidi cigarette, a cigarillo, a non-ventilated recess filter cigarette, a vented recess filter cigarette, a cigar, pipe tobacco, cigar tobacco, cigarette tobacco, chewing tobacco, moist snuff, nasal snuff, dry snuff, snus, leaf tobacco, hookah tobacco, shredded tobacco, and cut tobacco.

20. The tobacco product of embodiment 19, wherein the tobacco product is a smokeless tobacco product.

21. The tobacco product of embodiment 20, wherein the smokeless tobacco product is selected from the group consisting of loose leaf chewing tobacco, plug chewing tobacco, moist snuff, nasal snuff, dry snuff, and snus.

22. A reconstituted tobacco comprising the cured tobacco material of embodiment 16.

23. A tobacco product comprising material from a modified tobacco plant, tobacco seed, or tobacco plant part comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

24. A cannabis product comprising material from a modified cannabis plant, cannabis seed, or cannabis plant part comprising a recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

25. A recombinant nucleic acid molecule comprising a nucleic acid sequence encoding an amino acid sequence at least 80% identical or similar to an amino acid sequence selected from the group consisting of SEQ ID NOs: 18-22 and 31-33, wherein said nucleic acid sequence is operably linked to a heterologous promoter.

26. The recombinant nucleic acid molecule of embodiment 25, wherein said nucleic acid sequence encodes a protein at least 90% identical or similar to a sequence selected from the group consisting of SEQ ID NOs: 18-22 and 31-33.

27. The recombinant nucleic acid molecule of embodiment 25 or 26, wherein said nucleic acid sequence is at least 90% identical to a sequence selected from the group consisting of SEQ ID NOs: 13-17, 23-30, and 34-36.

28. The recombinant nucleic acid molecule of any one of embodiments 25-27, wherein said nucleic acid sequence encodes a protein at least 95% identical or similar to a sequence selected from the group consisting of SEQ ID NOs: 18-22 and 31-33.

29. The recombinant nucleic acid molecule of any one of embodiments 25-28, wherein said nucleic acid sequence is selected from the group consisting of SEQ ID NOs: 13-17, 23-30, and 34-36.

30. The recombinant nucleic acid molecule of any one of embodiments 25-29, wherein said nucleic acid sequence encodes a protein selected from the group consisting of SEQ ID NOs: 18-22 and 31-33.

31. A method for producing a plant, the method comprising:
(a) obtaining at least one plant comprising a recombinant nucleic acid molecule comprising a nucleic acid sequence at least 80% identical to a sequence selected from the group consisting of SEQ ID NOs: 13-17, 23-30, and 34-36, wherein said nucleic acid sequence is operably linked to a heterologous promoter;
(b) crossing said at least one plant with at least one plant of a second variety to produce at least one progeny seed; and
(c) selecting said at least one progeny seed produced in step (b), or a plant germinated therefrom, wherein said at least one progeny seed or plant germinated therefrom comprises said recombinant nucleic acid molecule.

32. The method of embodiment 31, further comprising
(d) harvesting plant material from a plant germinated from said at least one progeny seed; and
(e) producing a product comprising of or derived from said harvested plant material.

33. The method of embodiment 31 or 32, wherein a plant grown from said at least one progeny seed further comprises an amount of at least one terpene that is increased by at least 5% as compared to a control plant lacking said recombinant nucleic acid molecule.

34. The method of any one of embodiments 31-33, wherein a plant grown from said at least one progeny seed further comprises an amount of at least one terpene that is increased by at least 10% as compared to a control plant lacking said recombinant nucleic acid molecule.

35. The method of any one of embodiments 31-34, wherein said at least one plant is from a genus selected from the group consisting of Nicotiana and Cannabis.

36. The method of any one of embodiments 31-35, wherein said at least one plant is from a genus selected from the group consisting of tobacco and cannabis.

37. The method of any one of embodiments 31-36, wherein said a recombinant nucleic acid molecule comprises a nucleic acid sequence encoding a protein at least 80% identical or similar to a sequence selected from the group consisting of SEQ ID NOs: 18-22 and 31-33.

38. The method of any one of embodiments 31-37, wherein said a recombinant nucleic acid molecule comprises a nucleic acid sequence at least 90% identical to a sequence selected from the group consisting of SEQ ID NOs: 13-27 and 34-36.

39. The method of any one of embodiments 31-38, wherein said a recombinant nucleic acid molecule comprises a nucleic acid sequence encoding a protein at least 90% identical or similar to a sequence selected from the group consisting of SEQ ID NOs: 18-22 and 31-33.

40. The method of any one of embodiments 31-39, wherein said a recombinant nucleic acid molecule comprises a nucleic acid sequence selected from the group consisting of SEQ ID NOs: 13-27 and 34-36.

41. The method of any one of embodiments 31-40, wherein said a recombinant nucleic acid molecule comprises a nucleic acid sequence encoding a protein selected from the group consisting of SEQ ID NOs: 18-22 and 31-33.

42. A method of generating a modified plant, the method comprising:
(a) introducing a recombinant nucleic acid molecule comprising a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell;
(b) selecting at least one plant cell from step (a), wherein the at least one plant cell comprises the recombinant nucleic acid molecule; and
(c) regenerating a modified plant from the at least one plant cell selected in step (b).

43. A method comprising preparing a tobacco product using cured tobacco material from a modified tobacco plant or part therefrom, wherein the modified tobacco plant or part therefrom comprises a recombinant nucleic acid molecule comprises a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell.

44. A method comprising preparing a cannabis product using material from a modified cannabis plant or part therefrom, wherein the modified cannabis plant or part therefrom comprises a recombinant nucleic acid molecule comprises a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell.

45. A method comprising transforming a plant cell with a recombinant nucleic acid molecule, wherein the recombinant nucleic acid molecule comprises a nucleic acid sequence at least 80% identical to a sequence selected from the group consisting of SEQ ID NOs: 13-17, 23-30, and 34-36, wherein said nucleic acid sequence is operably linked to a heterologous promoter, to at least one plant cell.

46. A method for producing a plant with increased trichome density, the method comprising:
(a) providing to at least one plant cell a recombinant nucleic acid comprising a heterologous promoter operably linked to a polynucleotide, where the polynucleotide encodes an amino acid sequence at least 80% identical or similar to an amino acid sequence selected from the group consisting of SEQ ID NOs: 18-22 and 31-33; and
(b) regenerating a modified plant from the at least one plant cell in step (a), where the modified plant comprises an increased trichome density on at least one leaf as compared to a leaf from a control plant lacking the recombinant nucleic acid when grown under comparable conditions.

Having now generally described the disclosure, the same will be more readily understood through reference to the following examples that are provided by way of illustration, and are not intended to be limiting of the present disclosure, unless specified.

### EXAMPLES

### Example 1. Identification of Candidate Transcription Factors for Trichome Density Modification

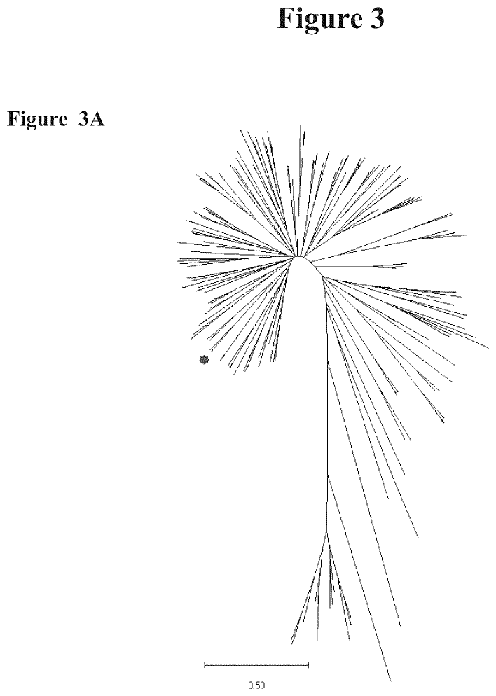

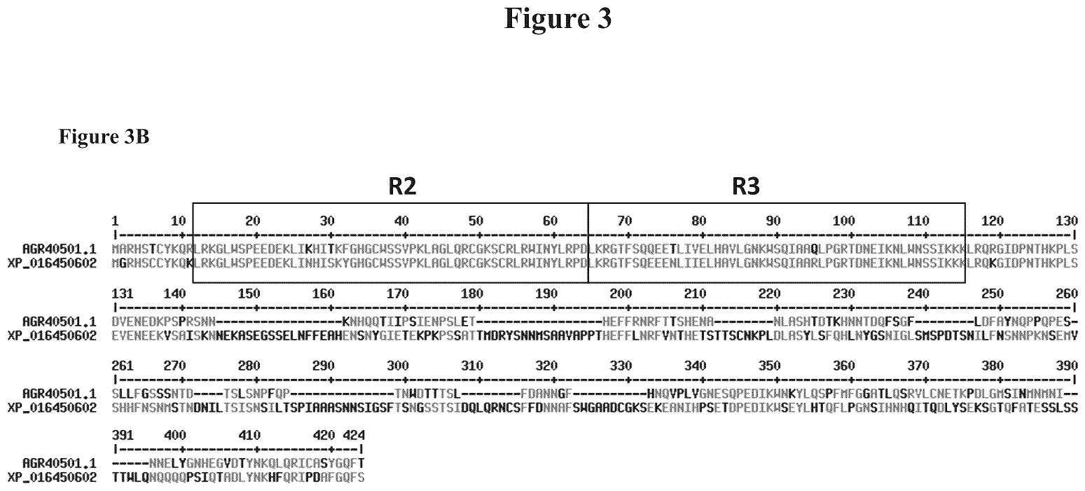

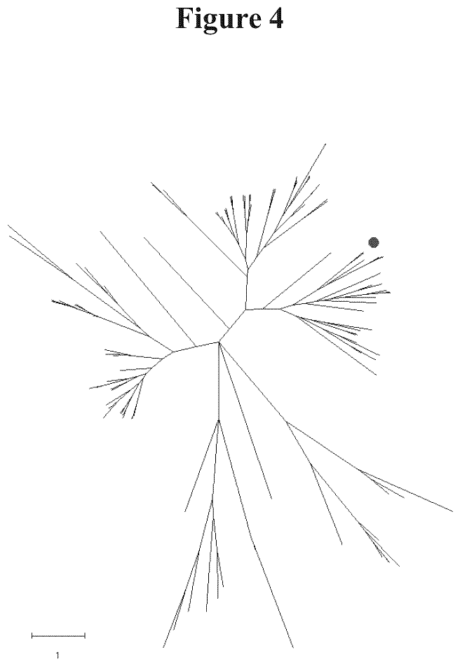

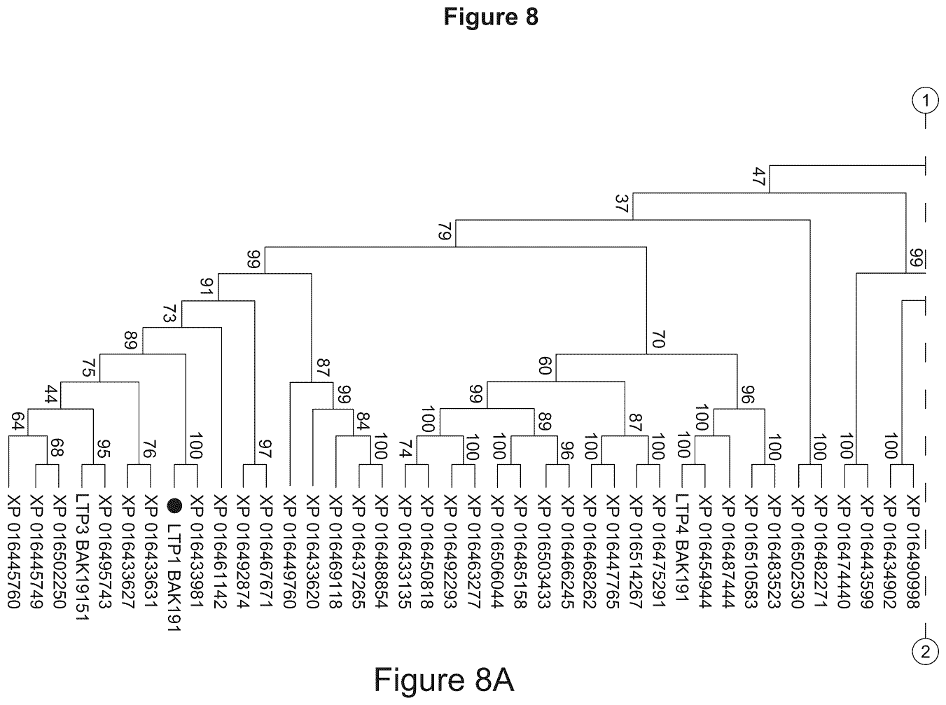

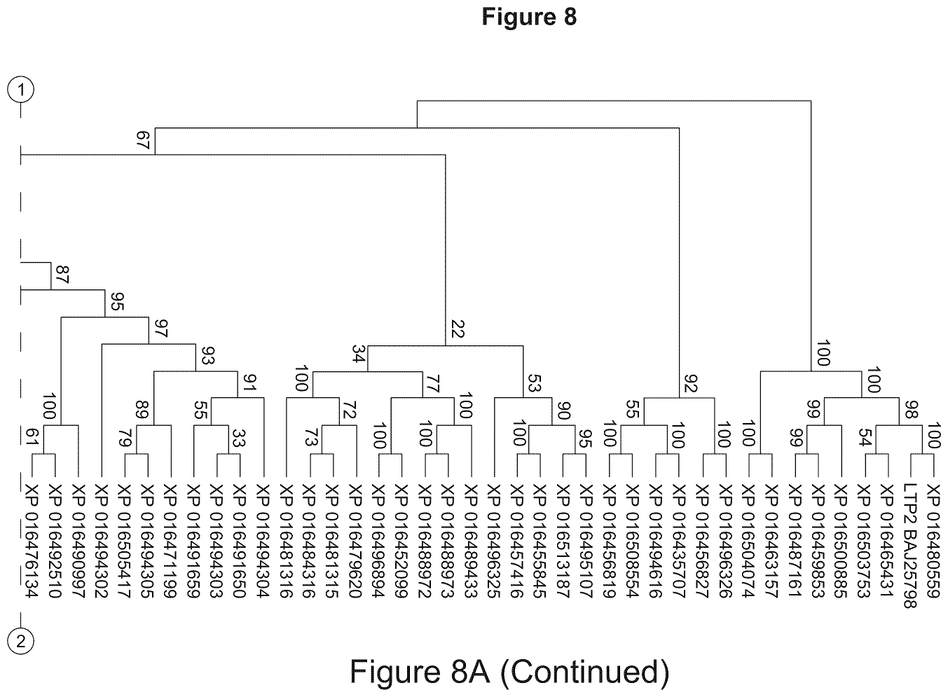

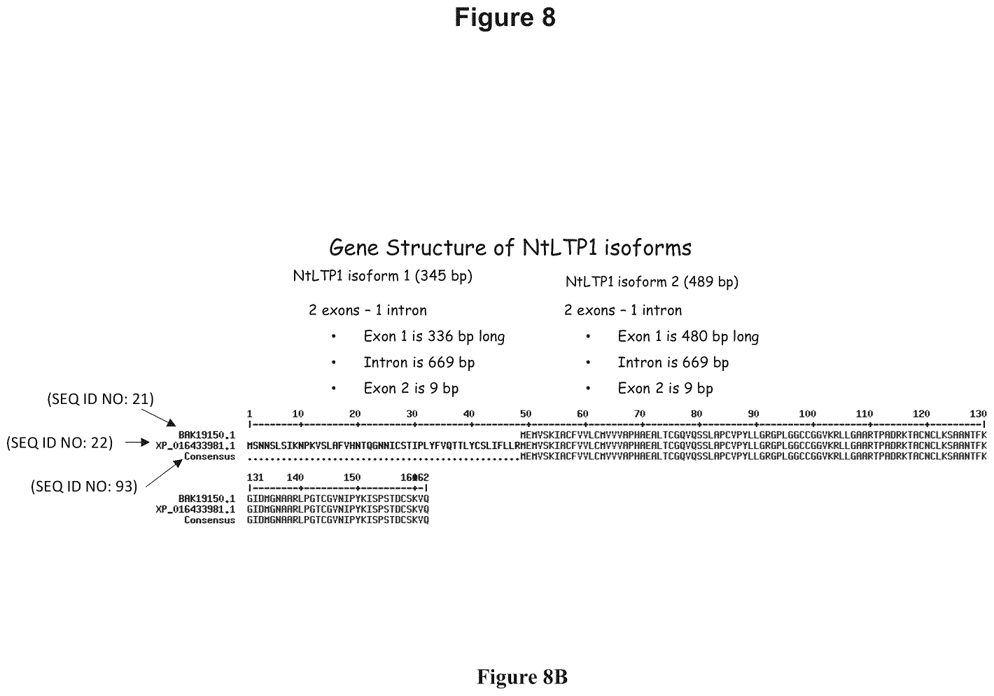

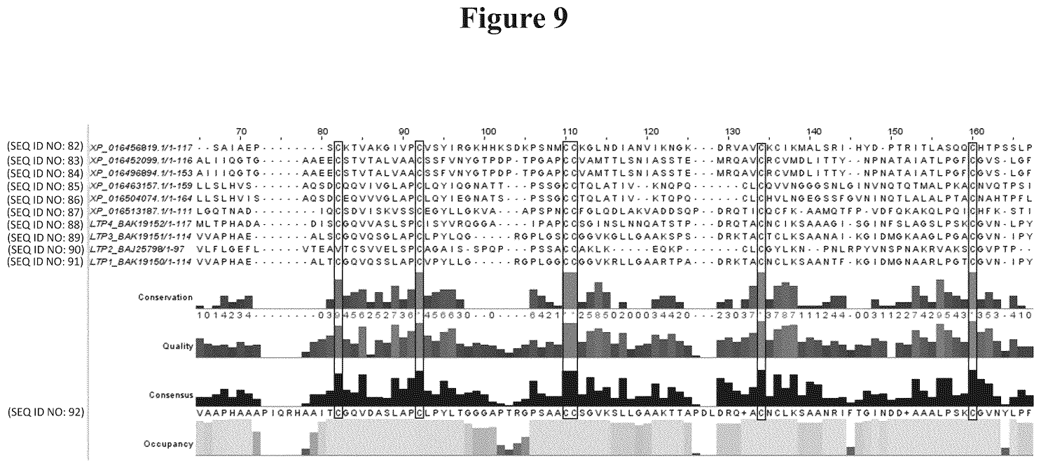

Transcription factors involved in regulation of trichome initiation and development were used as query in a search of the National Center for Biotechnology Information (NCBI) database. A similar approach is utilized to identify lipid transfer proteins in tobacco. The resulting sequences were then extracted from a second, internal database. This methodology identified members of the transcription factor families MYB (FIGS. 3A and 3B) and C2H2 (FIG. 4). From these families, three transcription factors candidates are selected for modification of trichome density in plants. This approach also identified 86 members of the lipid transfer protein (LTP) family in tobacco and subsequently led to identification of two variant isoforms of LTP1, isoform two (SEQ ID NO:27) having a 5′ extension compared to isoform 1 (SEQ ID NO:26). See FIGS. 8A and 8B. This approach allowed the curation of a list of candidate genes that are trichome-specific and involved in metabolite transport (SEQ ID NOs: 13-27 and 34-36). See Table 1.

### Example 2. Construction of Vectors

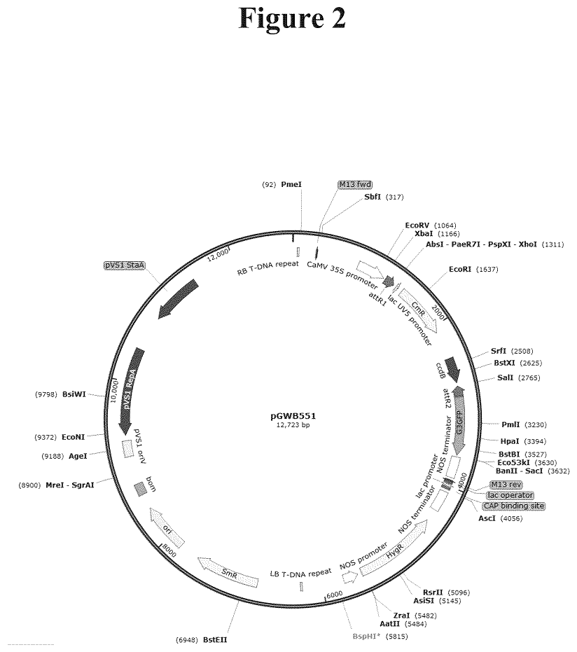

The candidate genes identified in Example 1 (e.g., SEQ ID NOs: 13-27 and 34-36) are cloned using the PCR Cloning System with GATEWAY™ Technology (ThermoFisher Scientific, Catalog Number 12535029). The cloned genes are subsequently subcloned into a GATEWAY™ expression vector, where the subcloned gene is fused with GREEN FLUORESCENCE PROTEIN (G3GFP) operably linked to a CaMV 35S promoter. See FIG. 2.

### Example 3. Transformation and Regeneration of Modified Tobacco Plants

Each of the vector constructs generated in Example 2 is separately transformed into tobacco cells in separate experiments. Briefly, the vectors are introduced into tobacco leaf discs via Agrobacterium transformation. See, for example, Mayo et al., Nat. Protoc., 1:1105-1111 (2006); and Horsch et al., Science, 227:1229-1231 (1985).

Transformed tobacco plants (variety ‘TN90’ and ‘Izmir Ego’) and N. benthamiana are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed, and the Agrobacterium tumefaciens cell pellet is re-suspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog (MS) with B5 vitamin liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (½ MS medium) and discs are placed upside down in contact with filter paper overlaid on the co-cultivation TOM medium (MS medium with 30 g/L sucrose; 0.1 mg/L 1-napthaleneacetic acid (NAA); and 1 mg/L 6-benzyl aminopurine (BAP)). The Petri plate is sealed with parafilm and incubated in the dark for two days.

After incubation, leaf discs are transferred to regeneration/selection TOM K medium Petri plates (TOM medium plus 200 mg/L cefotaxime and 50 mg/L hygromycin). Calli formed from leaf discs are sub-cultured bi-weekly to fresh TOM-Hyg medium in dim light (between 60 and 80 mE/ms) with photoperiods of 18 hours light, 6 hours dark, at 24° C. until shoots (plantlets) become excisable. Plantlets formed from calli are removed with forceps and subculture into MS rooting medium (MS medium with 3 g/L sucrose, 7 g/L dextrose with 200 mg/L cefotaxime and 50 mg/L hygromycin). Shoots on MS basal medium with 50 mg/L hygromycin are incubated with the same lighting (approximately 60-80 mE/ms) with photoperiods of 18 hours light, 6 hours dark, at 24° C. to induce rooting.

When plantlets comprising both shoots and roots grow large enough (e.g., over half the height of a Magenta™ GA-7 box), they are transferred to Jiffy peat pellets for acclimatization in the growth room. Once established, seedlings are transferred to a greenhouse for further growth, breeding, and analysis.

### Example 4. Confirming Expression of Candidate Genes in Modified Tobacco Plants

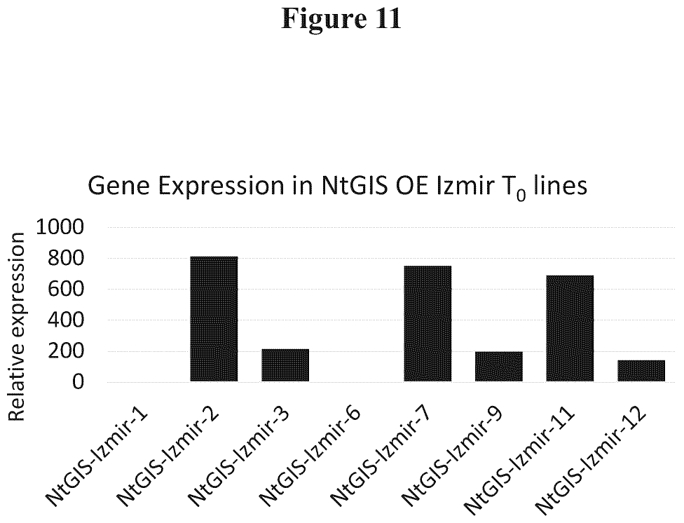

During the vegetative stage of growth, RNA is extracted from young leaves of modified tobacco plants produced in Example 3, and from control tobacco plants lacking the recombinant nucleic acid constructs grown under comparable conditions. The extracted RNA is used to generate cDNA. Gene expression of NtMYB and NtGIS is quantified using quantitative real-time PCR (qRT-PCR) in T₀ transgenic plants (see FIGS. 10 and 11). To confirm the constructs are expressing the recombinant nucleic acids, expression of NtMYB and NtGIS in the modified plants is compared to control tobacco plants.

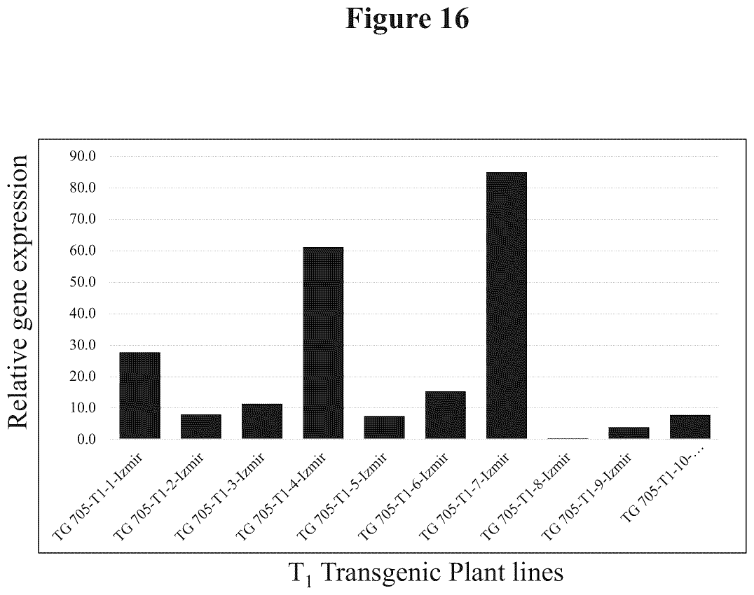

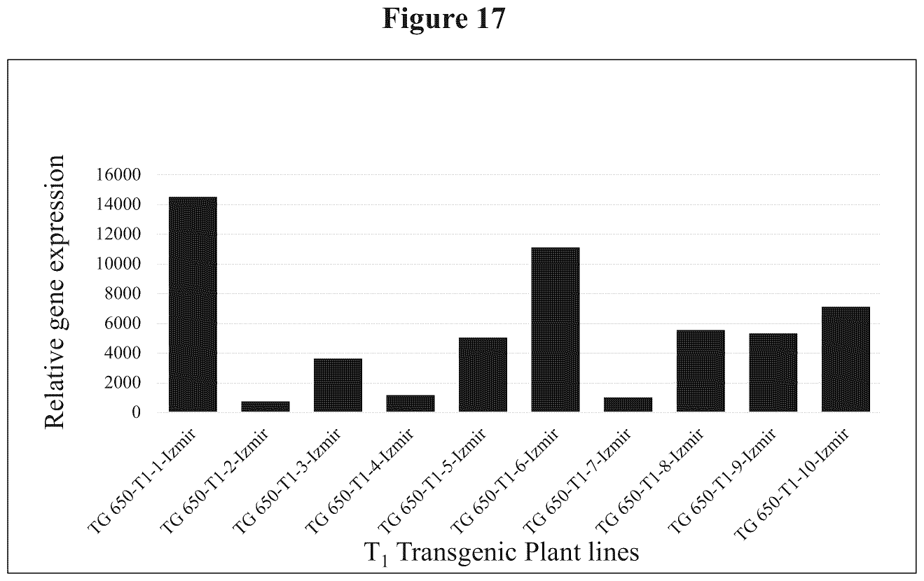

Gene expression of NtMYB and NbGIS is also quantified in T₁ transgenic plants. See FIGS. 16 and 17, respectively.

### Example 5. Confirming Modification of Trichome Density in Modified Tobacco Plants

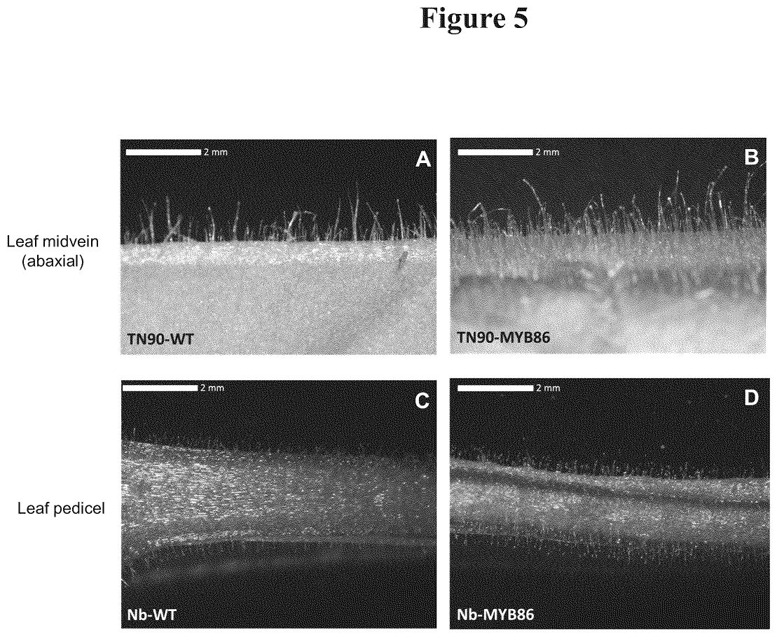

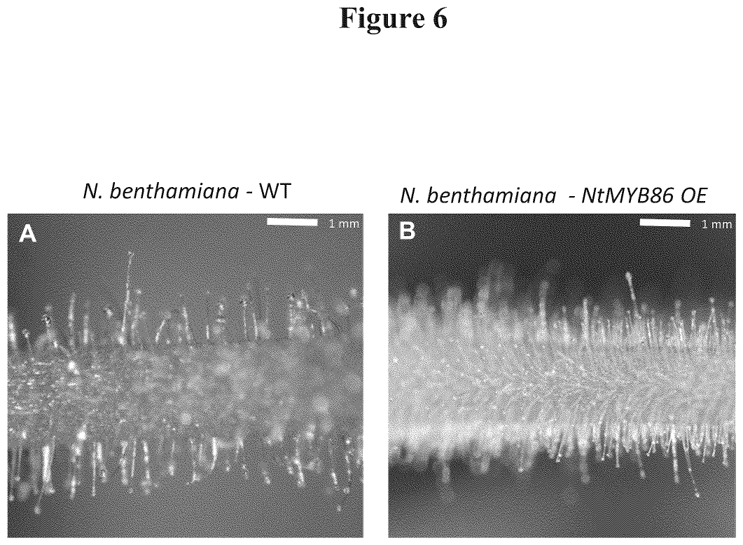

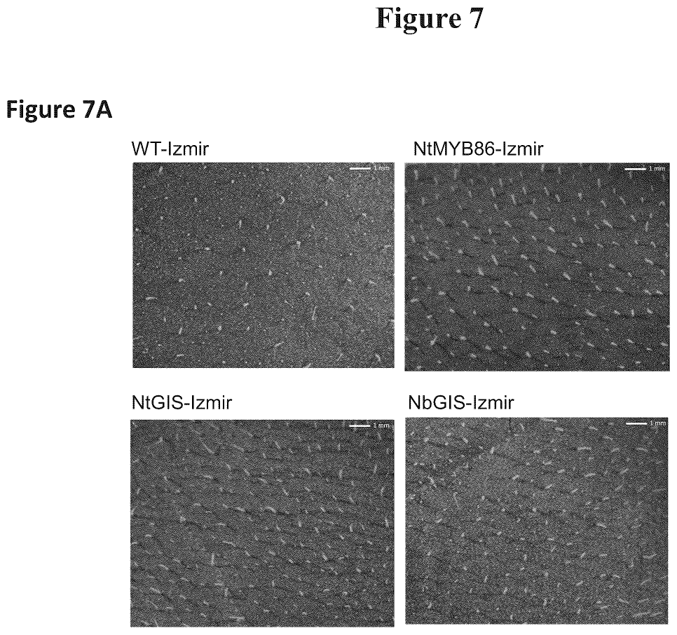

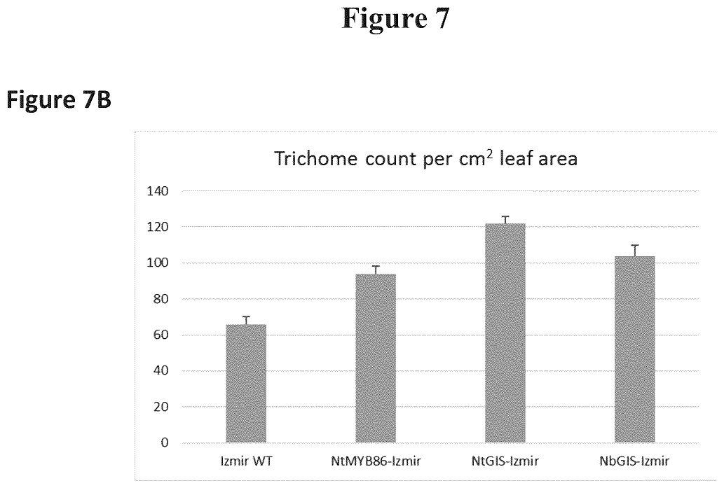

During the vegetative stage of growth, samples of transgenic lines of NtMYB86, NtGIS, and NbGIS from various tobacco backgrounds are examined under a stereomicroscope (model/type) to identify changes in trichome density in various plant parts compared with control plants. The results demonstrate that transgenic plants comprising NtMYB86, NtGIS, or NbGIS show an increase in the number of glandular trichomes per unit area (i.e. increased trichrome density) in leaf and stem tissues. (See FIGS. 5-7)

### Example 6. Measuring Terpene Levels in Modified Tobacco Plants

During the vegetative stage of growth, young leaves are harvested from the modified tobacco plants from Examples 3, 4, and 5, and from control tobacco plants lacking the recombinant nucleic acid constructs grown under comparable conditions, for use in a qualitative metabolic profile analysis following the protocol outlined by Jiang et al. (Curr Protoc Plant Biol. 2016; 1:345-358). Leaf samples are ground in liquid nitrogen, and then the samples are mixed with 60:40 hexane:ethyl acetate (v/v), supplemented with heptadecanol (an internal standard) and incubated overnight with shaking.

The solvent extracts are concentrated in a refrigerated SpeedVac™ (ThermoFisher Scientific) and placed into a silica column. The column is washed with hexane and allowed to flow through into collection tubes. Samples are aliquoted from the collection tubes and used for gas chromatography-mass spectrometry (GC-MS) analysis of metabolites.

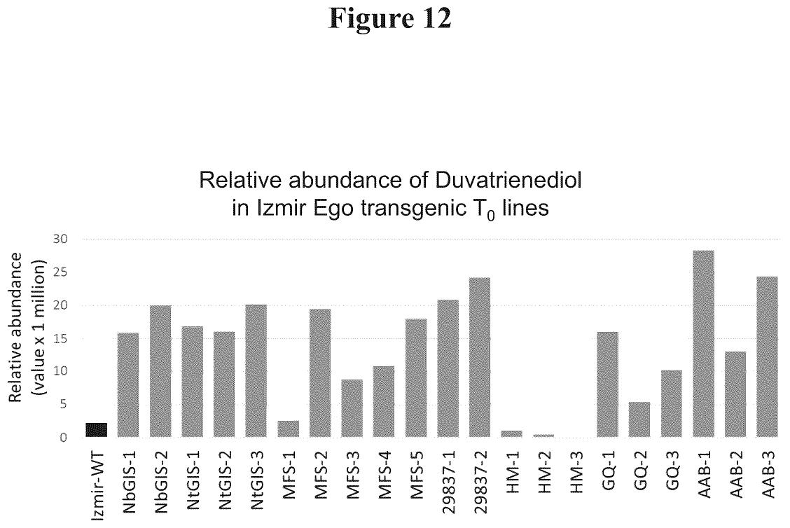

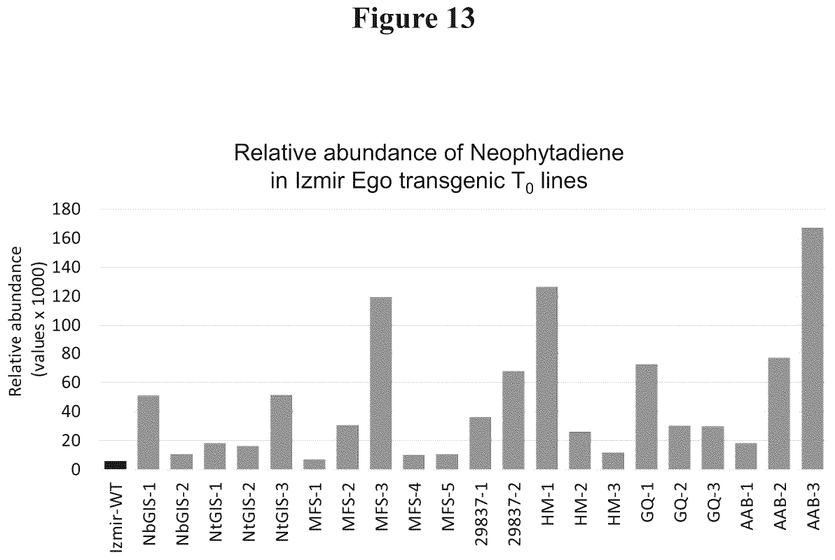

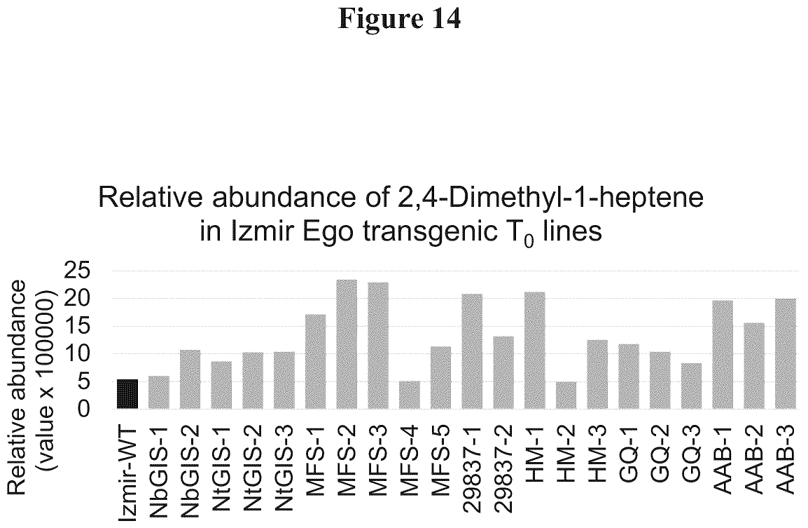

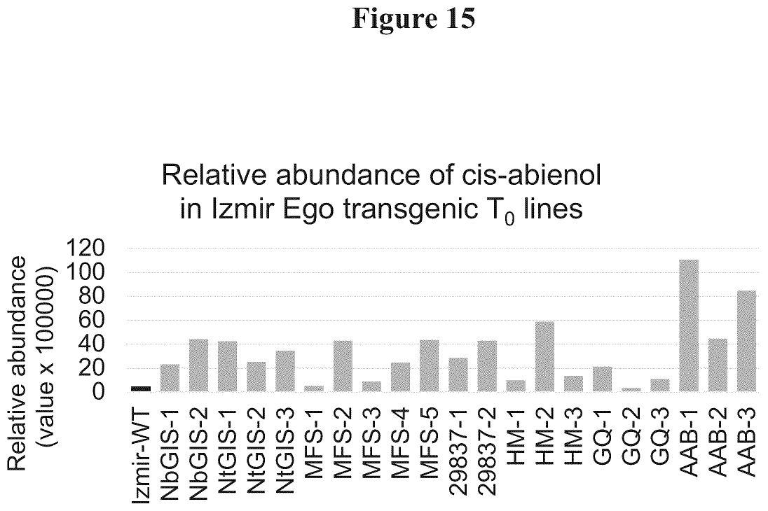

To identify secondary metabolites secreted from leaf trichomes, 1 gram of leaf samples from transgenic lines are cut into small sections and soaked into hexane supplemented with heptadecanol (an internal standard) and incubated overnight with shaking. The extract is filtered and used for analysis of metabolic profile in gas chromatography-mass spectrometry (GC-MS). See FIGS. 12-15.

1-46. (canceled)

47. A modified tobacco plant, tobacco seed, or tobacco plant part comprising a recombinant nucleic acid molecule comprising heterologous promoter operably linked to a nucleic acid sequence encoding an amino acid sequence at least 95% identical to the amino acid sequence of SEQ ID NO: 19, and wherein at least one leaf of the modified tobacco plant comprises a greater average trichome density as compared to a leaf of a control tobacco plant grown under comparable conditions.

48. The modified tobacco plant, tobacco seed, or tobacco plant part of claim 47, wherein the amino acid sequence is identical to the amino acid sequence of SEQ ID NO: 19.

49. The modified tobacco plant, tobacco seed, or tobacco plant part, of claim 47, wherein the nucleic acid sequence is at least 95% identical to the nucleic acid sequence of SEQ ID NO: 14 or SEQ ID NO: 24.

50. The modified tobacco plant, tobacco seed, or tobacco plant part, of claim 47, wherein the nucleic acid sequence is identical to the nucleic acid sequence of SEQ ID NO: 14 or SEQ ID NO: 24.

51. The modified tobacco plant, tobacco seed, or tobacco plant part thereof of claim 47, wherein the tobacco plant, tobacco seed, or tobacco plant part is of a tobacco variety selected from the group consisting of a flue-cured variety, a bright variety, a Burley variety, a Virginia variety, a Maryland variety, a dark variety, a Galpão variety, an Oriental variety, and a Turkish variety.

52. The modified tobacco plant, tobacco seed, or tobacco plant part thereof of claim 47, wherein the modified tobacco plant is male sterile or cytoplasmically male sterile.

53. The modified tobacco plant, tobacco seed, or tobacco plant part, of claim 47, wherein the heterologous promoter is selected from the group consisting of a constitutive promoter, an inducible promoter, a tissue-preferred promoter, and a tissue-specific promoter.

54. The modified tobacco plant, tobacco seed, or tobacco plant part of claim 47, wherein at least one leaf of the modified tobacco plant comprises a greater average trichome density on the abaxial side of the at least one leaf of the modified tobacco plant as compared to the abaxial side of a leaf of a control tobacco plant grown under comparable conditions.

55. The modified plant, seed, or plant part of claim 47, wherein at least one leaf of the modified tobacco plant comprises a greater average trichome density on the adaxial side of the at least one leaf of the modified tobacco plant as compared to the adaxial side of a leaf of a control tobacco plant grown under comparable conditions.

56. The modified tobacco plant, tobacco seed, or tobacco plant part of claim 47, wherein the greater average trichome density comprises a greater average density of glandular trichomes.

57. The modified tobacco plant, tobacco seed, or tobacco plant part of claim 47, wherein the at least one leaf of the modified tobacco plant comprises at least 110 glandular trichomes per square centimeter.

58. Cured tobacco material from a modified tobacco plant or tobacco plant part comprising a recombinant nucleic acid molecule comprising a heterologous promoter operably linked to a nucleic acid sequence encoding an amino acid sequence least 95% identical to the amino acid sequence of SEQ ID NO: 19, and wherein at least one leaf of the modified tobacco plant comprises a greater average trichome density as compared to a leaf of a control tobacco plant grown under comparable conditions.

59. The cured tobacco material of claim 58, wherein the amino acid sequence is identical to the amino acid sequence of SEQ ID NO: 19.

60. The cured tobacco material of claim 58, wherein the nucleic acid sequence is at least 95% identical to the nucleic acid sequence of SEQ ID NO: 14 or SEQ ID NO: 24.

61. The cured tobacco material of claim 58, wherein the cured tobacco material is selected from the group consisting of flue-cured tobacco material, air-cured tobacco material, fire-cured tobacco material, and sun-cured tobacco material.

62. The cured tobacco material of claim 58, wherein the cured tobacco material is selected from the group consisting of cured leaf material and cured stem material.

63. A tobacco product comprising the cured tobacco material of claim 58.

64. The tobacco product of claim 63, wherein the tobacco product is selected from the group consisting of a kretek, a bidi cigarette, a cigarillo, a non-ventilated recess filter cigarette, a vented recess filter cigarette, a cigar, pipe tobacco, cigar tobacco, cigarette tobacco, chewing tobacco, moist snuff, nasal snuff, dry snuff, snus, leaf tobacco, hookah tobacco, shredded tobacco, and cut tobacco.

65. The tobacco product of claim 63, wherein the tobacco product is a smokeless tobacco product.

66. The tobacco product of claim 65, wherein the smokeless tobacco product is selected from the group consisting of loose leaf chewing tobacco, plug chewing tobacco, moist snuff, nasal snuff, dry snuff, and snus.
